(19) United States
(12) Patent Application Publication
(10) Pub. No.: US 2008/0119496 A1
(43) Pub. Date: May 22, 2008

(54) 7-Substituted Purine Derivatives for Immunosuppression

(75) Inventors: Michael J. Ohlmeyer, Plainsboro, NJ (US); Adolph Bohnstedt, Burlington, NJ (US); Celia Kingsbury, Cream Ridge, NJ (US); Koc-Kan Ho, West Windsor, NJ (US); Jorge Quintero, Sayreville, NJ (US)

Correspondence Address: HESLIN ROTHENBERG FARLEY & MESITI PC, 5 COLUMBIA CIRCLE, ALBANY, NY, 12203, US

(73) Assignee: PHARMACOPEIA DRUG DISCOVERY, INC., Cranbury, NJ (US)

(21) Appl. No.: 11/560,731

(22) Filed: Nov. 16, 2006

(51) Int. Cl. A61K 31/522; A61K 31/437; A61P 17/12; A61P 37/00; A61P 43/00; C07D 471/04; C07D 473/28

(52) U.S. Cl. 514/263.21; 514/303; 544/276; 546/118

(57) ABSTRACT

The present invention provides novel purinone and related derivatives useful for the prevention and treatment of autoimmune diseases, inflammatory disease, mast cell mediated disease and transplant rejection. The compounds are of the general formula III:

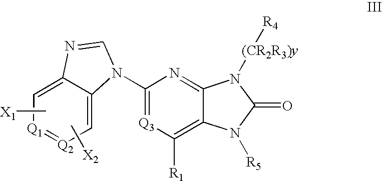

### FIELD OF THE INVENTION

The invention relates to purinone derivatives useful as immunosuppressants.

### BACKGROUND OF THE INVENTION

Immunosuppression is an important clinical approach in treating autoimmune disease and in preventing organ and tissue rejection. The clinically available immunosuppressants, including azathioprine, cyclosporine and tacrolimus, although effective, often cause undesirable side effects including nephrotoxicity, hypertension, gastrointestinal disturbances and gum inflammation. Inhibitors of the tyrosine kinase Jak3 are known to be useful as immunosuppressants (see U.S. Pat. No. 6,313,129).

The members of the Janus kinase (Jak) family of non-receptor intracellular tyrosine kinases are components of cytokine signal transduction. Four family members have been identified to date: Jak1, Jak2, Jak3 and Tyk2. The Jaks play a key role in the intracellular signaling mediated through cytokine receptors. Upon binding of cytokines to their receptors, Jaks are activated and phosphorylate the receptors, creating docking sites for other signaling molecules, in particular members of the signal transducer and activator of transcription (STAT) family. While expression of Jak1, Jak2 and Tyk2 is relatively ubiquitous, Jak3 expression is temporally and spatially regulated. Jak3 is predominantly expressed in cells of hematopoietic lineage; it is constitutively expressed in natural killer (NK) cells and thymocytes and is inducible in T cells, B cells and myeloid cells (reviewed in Ortmann, et al., 1999 and Yamaoka, et al., 2004). Jak3 is also is expressed in mast cells, and its enzymatic activity is enhanced by IgE receptor/FcεRI cross-linking (Malaviya and Uckun, 1999).

A specific, orally active Jak3 inhibitor, CP-690,550, has been shown to act as an effective immunosuppressant and prolong animal survival in a murine model of heart transplantation and a primate model of kidney transplantation (Changelian, et al., 2003).

Furthermore, aberrant Jak3 activity has been linked to a leukemic form of cutaneous T-cell lymphoma (Sezary's syndrome) and acute lymphoblastic leukemia (ALL), the most common form of childhood cancer. The identification of Jak3 inhibitors has provided the basis for new clinical approaches in treating leukemias and lymphomas (reviewed in Uckun, et al, 2005). Two dimethoxyquinazoline derivatives, WHI-P131 (JANEX-1) and WHI-P154 (JANEX-2), have been reported to be selective inhibitors of Jak3 in leukemia cells (Sudbeck et al., 1999).

Jak3 has also been shown to play a role in mast-cell mediated allergic reactions and inflammatory diseases and serves as a target in indications such as asthma and anaphylaxis.

Therefore, compounds that inhibit Jak3 are useful for indications such as leukemias and lymphomas, organ and bone marrow transplant rejection, mast cell-mediated allergic reactions and inflammatory diseases and disorders.

### SUMMARY OF THE INVENTION

It has now been found that compounds of general formula III are potent and selective inhibitors of Jak3:

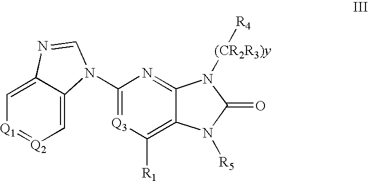

In these compounds,

### Q₁ and Q₂ are independently selected from the group consisting of CX₁, CX₂ and nitrogen wherein Q₁ and Q₂ are not both nitrogen;

### Q₃ is N or CH;

### X₁ and X₂ are independently selected from the group consisting of hydrogen, (C₁-C₆)alkyl, cyano, halo, halo(C₁-C₆)alkyl, hydroxyl, (C₁-C₆)alkoxy; halo(C₁-C₆)alkoxy, and nitro;

### R₁ is selected from the group consisting of hydrogen and (C₁-C₆)alkyl;

y is zero or an integer selected from 1, 2 and 3;

### R₂ and R₃ are selected independently for each occurrence of (CR₂R₃) from the group consisting of hydrogen and (C₁-C₆)alkyl;

### R₄ is selected from a group consisting of alkyl, heterocyclyl, aryl, heteroaryl, substituted alkyl, substituted heterocyclyl, substituted aryl, substituted heteroaryl; and

### R₅ is selected from the group consisting of alkyl, heterocyclyl, heterocyclyl, and C₁-C₆ alkyl wherein

(a) one or two CH₂ is replaced by a group chosen from NH and N(alkyl);
(b) one or two CH₂ is replaced by O;
(c) one or two CH₂ is replaced by (C═O);
(d) two CH₂ are replaced by CH═CH or C≡C; or
(e) any chemically stable combination of (a), (b) (c) and (d);
and wherein from zero to three hydrogens is replaced by a substituent chosen from:
(a) halogen, hydroxy, cyano, loweralkylsulfonyl, loweralkylsulfonyloxy, amino, loweralkylamino, diloweralkylamino, alkoxyamino, sulfonylamino, acylamino, arylamino, loweralkoxy;
(b) heterocyclyl and heterocyclyl substituted with from one to three substituents chosen from halogen, hydroxy, alkoxy, alkyl and alkoxycarbonyl;
(c) phenyl and phenyl substituted with from one to three substituents chosen from halogen, hydroxy, alkoxy, alkyl, acylamino, cyano, carboxy, alkoxycarbonyl, haloalkyl and heterocyclyl; and
(d) heteroaryl and heteroaryl substituted with from one to three substituents chosen from halogen, hydroxy, alkoxy, alkyl and alkoxycarbonyl.

The members of these genera are useful in inhibiting Jak3 activity and as such are useful in indications where clinical immunosuppression is desired and in the treatment of hematological cancers. The compounds are more selective for Jak3 than for Aurora A kinases than are the corresponding compounds in which R₅ is hydrogen.

In another aspect, the invention relates to pharmaceutical compositions comprising a therapeutically effective amount of at least one compound of general formula III, or a pharmaceutically acceptable salt thereof; and a pharmaceutically acceptable carrier.

In another aspect, the invention relates to a method for treating a disease by altering a response mediated by Jak3 tyrosine kinase. The method comprises bringing into contact with Jak3 at least one compound of general formula III.

In yet another aspect the present invention relates to a method of suppressing the immune system in a subject in need thereof comprising administering to the subject a therapeutically effective amount of at least one compound of general formula III.

In a further aspect of the present invention relates to a method for treating a disease or disorder selected from an autoimmune disease, an inflammatory disease, a mast cell mediated disease, hematological malignancy and organ transplant rejection in a subject in need thereof comprising administering to a subject a therapeutically effective amount of at least one compound of general formula III.

Suppression of immune system activity is desirable for preventing or treating tissue or organ rejection following transplant surgery and for preventing and treating diseases and disorders arising from aberrant activity of the immune system, in particular autoimmune disorders and diseases. Exemplary autoimmune disorders include graft versus host disease (GVHD), insulin-dependent diabetes (Type I), Hashimoto's thyroiditis and Graves' disease, pernicious anemia, Addison's disease, chronic active hepatitis, Crohn's disease, ulcerative colitis, rheumatoid arthritis, multiple sclerosis, systemic lupus erythematosus, psoriasis, scleroderma and myasthenia gravis.

The compounds of the present invention are useful in preventing and treating diseases and disorders related to mast cell-mediated allergic reactions and inflammation, such as keratoconjuctivitis sicca.

Other indications in which the Jak3 inhibitors are useful include leukemias and lymphomas.

### DETAILED DESCRIPTION OF THE INVENTION

Throughout this specification the substituents are defined when introduced and retain their definitions.

In a first aspect the invention relates to purinones and imidazopyridinones having general formula III:

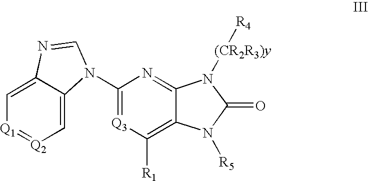

The members of the genus III may be conveniently divided into subgenera based on the values of Q. When Q₁ is nitrogen and Q₂ is carbon, a subgenus of purinones and imidazo[4,5-b]pyridinones having an attached imidazo[4,5-c]pyridine arises. When Q₁ is carbon and Q₂ is nitrogen, a subgenus of purinones and imidazo[4,5-b]pyridinones having an attached imidazo[5,4-c]pyridine arises. When Q₁ and Q₂ are both carbon, a subgenus of purinones and imidazo[4,5-b]pyridinones having an attached benzimidazole arises. The genus could similarly be divided on the basis of Q₃. When Q₃ is nitrogen, a subgenus of purinones having an attached imidazo[4,5-c]pyridine, imidazo[5,4-c]pyridine or benzimidazole arises. When Q₃ is carbon, a subgenus of imidazo[4,5-b]pyridinones having an attached imidazo[4,5-c]pyridine, imidazo[5,4-c]pyridine or benzimidazole arises. The structures of these subgenera are shown below:

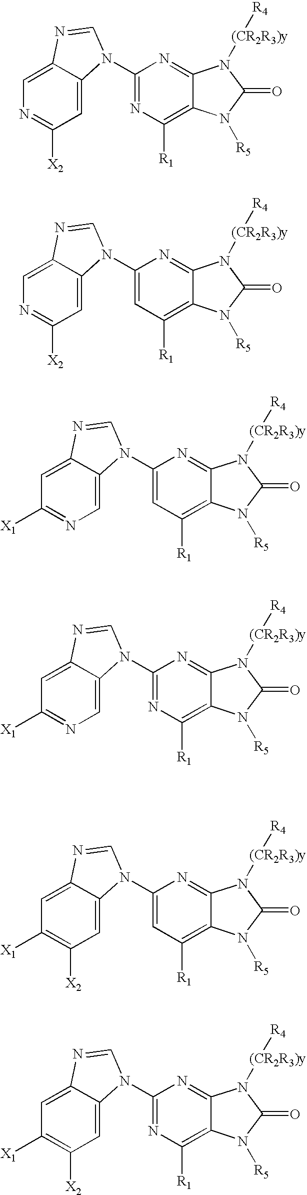

In certain embodiments, X₁ and X₂ are selected from hydrogen, cyano, chloro, fluoro, trifluoromethyl, trifluoromethoxy and methyl; in other embodiments R₁ is H. In one subdivision, y is zero; in another y is 1 or 2 and R₂ and R₃ are hydrogen or methyl. Examples of R₄ include: cyclopentyl, cyclohexyl, piperidine, oxepane, benzoxepane, dihydrocyclopentapyridine, phenyl, benzyl, tetralin, indane, tetrahydropyran, tetrahydrofuran, tetrahydroindole, isoquinoline, tetrahydroisoquinoline, quinoline, tetrahydroquinoline, chroman, pyridine, pyrimidine, pyrazine, dihydropyran, dihydrobenzofuran, tetrahydrobenzofuran, tetrahydrobenzothiophene, furan, dihydropyrano[2,3-b]pyridine (see example below), tetrahydroquinoxaline, tetrahydrothiopyran (thiane), thiochroman (dihydrobenzothiin) or any of the foregoing rings carrying from 1-3 additional substituents, such as halogen, methyl, methoxy, trifluoromethyl, cyano, hydroxy, oxo, oxide and acetyl. Additional examples in which R₄ is alkyl or substituted alkyl include subgenera in which R₄ is oxaalkyl (alkoxyalkyl).

In certain embodiments of genus III, y is 1 or 2; R₂ and R₃ are hydrogen or methyl and R₄ is phenyl, quinoline, pyridine, pyrazine or substituted phenyl, quinoline, pyridine or pyrazine. In other embodiments of genus III, y is zero and R₄ is cyclopentyl, cyclohexyl, phenyl, indane, piperidine, oxepane, benzoxepane, dihydrocyclopentapyridine, tetralin, tetrahydropyran, tetrahydrofuran, tetrahydroindole, isoquinoline, tetrahydroisoquinoline, quinoline, tetrahydroquinoline, chroman, pyridine, pyrimidine, dihydropyran, dihydrobenzofuran, tetrahydrobenzofuran, tetrahydrobenzothiophene, furan, dihydropyrano[2,3-b]pyridine, tetrahydroquinoxaline, tetrahydrothiopyran (thiane), thiochroman (dihydrobenzothiin) or a substituted ring from the foregoing list. In further embodiments, (a) y is zero and R₄ is selected from cyclohexyl, oxepane, tetralin, indane, dihydrocyclopentapyridine, tetrahydropyran, tetrahydroquinoline, chroman, dihydrobenzofuran, tetrahydrobenzofuran, dihydropyrano[2,3-b]pyridine and tetrahydroquinoxaline, each optionally substituted with hydroxy, oxo, or halogen; or (b) y is 1 or 2, R₂ and R₃ are hydrogen or methyl and R₄ is selected from phenyl, pyridine and pyrazine, each optionally substituted with halogen. When y is zero, R₄ may be tetrahydropyran-4-yl, 4-hydroxycyclohexyl, 4-oxocyclohexyl, oxepan-4-yl, chroman-4-yl; 3,4-dihydronaphthalen-1(2H)-on-4-yl; 2,3-dihydroinden-1-on-4-yl and their fluoro substituted counterparts. It appears that, although both enantiomers are active, compounds in which the carbon at 4 of the chroman is of the (R) configuration have higher potency. Certain of the foregoing subgenera in which y is zero may also be described by a representation in which R₄ is

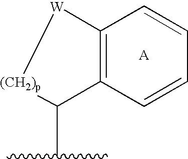

According to this representation, W is CH₂, C═O or O; p is 1, 2 or 3; and A is a six-membered heteroaromatic ring containing 1 or 2 nitrogens or a benzene ring optionally substituted with one or two fluorines. The wavy line denotes the point of attachment to the purinone. Compounds in which the carbon marked with an asterisk

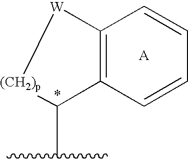

is of the (R) configuration appear to be more potent than their corresponding (S) enantiomers. Other embodiments include compounds in which y is 1 and R₄ is selected from difluorophenyl, fluorophenyl, chlorophenyl, chlorofluorophenyl, pyridin-3-yl and pyrazin-3-yl.

In certain embodiments of genus III, X₁ and X₂ are selected from hydrogen, cyano, chloro, fluoro, trifluoromethyl, trifluoromethoxy and methyl. In narrower embodiments, X₁ is selected from hydrogen, cyano and fluoro; Q₁ and Q₂ are CX₁; Q₃ is N and R₁ is H. In some embodiments, y is zero and R₄ is selected from cyclohexyl, tetralin, indane, oxepane, dihydrocyclopentapyridine, tetrahydropyran, tetrahydroquinoline, chroman, dihydrobenzofuran, tetrahydrobenzofuran, dihydropyrano[2,3-b]pyridine and tetrahydroquinoxaline, each optionally substituted with hydroxy, oxo, or halogen. In other embodiments y is 1 or 2, R₂ and R₃ are hydrogen or methyl and R₄ is selected from phenyl, pyridine and pyrazine, each optionally substituted with halogen.

In certain embodiments, R₅ is C₁-C₆ alkyl wherein certain replacements and substitutions have been made. In one embodiment, (a), one or two CH₂ may be replaced by NH or N(alkyl). These residues are also referred to as azaalkyl. An example of such an R₅ is —CH₂CH₂CH₂N(CH3)CH₂CN, which may be considered 5-cyanopentane in which one CH₂ has been replaced by N(CH₃). In one embodiment, (b), one or two CH₂ may be replaced by O. These residues are also referred to as oxaalkyl. In one embodiment, (c), one or two CH₂ may be replaced by (C═O). An example of such an R₅ is

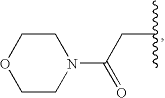

which may be considered a 2-(4-morpholinyl)ethyl in which one CH₂ has been replaced by C═O. In one embodiment, (d), two CH₂ are replaced by CH═CH or C≡C. An example of such an R₅ is 3-methylbut-2-ene-1-yl [—CH₂CH═C(CH₃)₂]. A further embodiment includes (e), any chemically stable combination of (a), (b) (c) and (d). An example of such an R₅ is —CH₂CH₂CH₂NHC(═O)CH₂CN, which may be considered 6-cyanohexane in which one CH₂ has been replaced by NH and one CH₂ has been replaced by C═O. The term “chemically stable” is readily understood by persons of skill in the chemical art. It encompasses compounds that may be isolated and purified and that either do not measurably decompose at physiological pH in aqueous solution or do so at a rate such that >99% remains after one hour at ambient temperature. In all of these compounds, from zero to three hydrogens may be replaced by a substituent chosen from:
(a) halogen, hydroxy, cyano, loweralkylsulfonyl, loweralkylsulfonyloxy, amino, loweralkylamino, diloweralkylamino, alkoxyamino, sulfonylamino, acylamino, arylamino, loweralkoxy;
(b) heterocyclyl and heterocyclyl substituted with from one to three substituents chosen from halogen, hydroxy, alkoxy, alkyl and alkoxycarbonyl;
(c) phenyl and phenyl substituted with from one to three substituents chosen from halogen, hydroxy, alkoxy, alkyl, acylamino, cyano, carboxy, alkoxycarbonyl, haloalkyl and heterocyclyl.

Some examples of the aforementioned substituents for C₁-C₆ alkyl include but are not limited to:

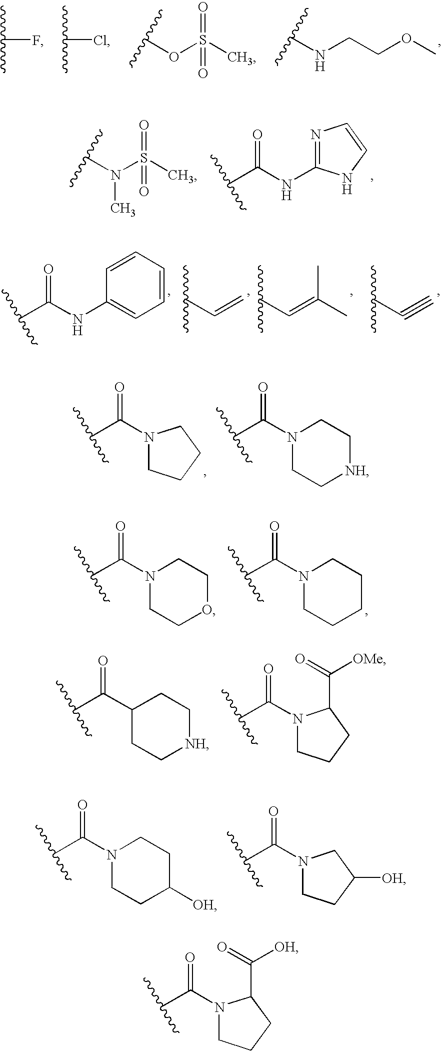

In certain embodiments of genus III, Q₁ is CX₁, Q₂ is CX₂, X₁ is hydrogen, X₂ is a substituent at the 6 position of the benzimidazole, and X₂ is chosen from hydrogen, fluoro and cyano.

All of the compounds falling within the foregoing parent genera and their subgenera are useful as Jak3 inhibitors.

### Definitions

For convenience and clarity certain terms employed in the specification, examples and claims are described herein.

Alkyl is intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, butyl, s- and t-butyl and the like. Preferred alkyl groups are those of C₂₀ or below; more preferred are C₁-C₈ alkyl. Cycloalkyl is a subset of alkyl and includes cyclic hydrocarbon groups of from 3 to 8 carbon atoms. Examples of cycloalkyl groups include cyclopropyl, cyclobutyl, cyclopentyl, cyclohexyl, norbornyl and the like.

C₁ to C₂₀ hydrocarbon includes alkyl, cycloalkyl, alkenyl, alkynyl, aryl and combinations thereof. Examples include phenethyl, cyclohexylmethyl, camphoryl and naphthylethyl.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds); it does not refer to doubly bonded oxygen, as would be found in carbonyl groups.

Acyl refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. The aromatic 6- to 14-membered carbocyclic rings include, e.g., benzene and naphthalene, and for the purposes of the present invention, fused moieties such as tetrahydronaphthalene (tetralin), indane and fluorine, in which one or more rings are aromatic, but not all need be. The 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl refers to a substituent in which an aryl residue is attached to the parent structure through alkyl. Examples are benzyl, phenethyl and the like. Heteroarylalkyl refers to a substituent in which a heteroaryl residue is attached to the parent structure through alkyl. Examples include, e.g., pyridinylmethyl, pyrimidinylethyl and the like.

Heterocycle means a cycloalkyl in which from one to three carbons is replaced by a heteroatom selected from the group consisting of N, O and S. The nitrogen and sulfur heteroatoms may optionally be oxidized. In some contexts (other than the present) the term heterocycle may be interpreted to include heteroaryl; for the purpose of this application, heterocycle is a saturated heterocycle and does not include heteroaryl. When heteroaryl is intended, it is expressly named. Examples of heterocycles include pyrrolidine, morpholine, dioxane, tetrahydrofuran, and the like. Examples of heterocyclyl residues additionally include piperazinyl, 2-oxopiperazinyl, 2-oxopiperidinyl, 2-oxo-pyrrolidinyl, 4-piperidinyl, pyrazolidinyl, oxazolidinyl, isoxazolidinyl, thiazolidinyl, quinuclidinyl, tetrahydrofuryl, tetrahydropyranyl, thiamorpholinyl, thiamorpholinylsulfoxide, and thiamorpholinylsulfone. A nitrogenous heterocycle is a heterocycle containing at least one nitrogen in the ring; it may contain additional nitrogens, as well as other heteroatoms.

Substituted alkyl, aryl, cycloalkyl, heterocyclyl, heteroaryl etc. refer to alkyl, aryl, cycloalkyl, heterocyclyl, or heteroaryl wherein up to three H atoms in each residue are replaced with halogen, haloalkyl, hydroxy, loweralkoxy, hydroxyloweralkyl, carboxy, carboalkoxy (also referred to as alkoxycarbonyl), carboxamido (also referred to as alkylaminocarbonyl), heterocyclylcarbonyl, cyano, carbonyl, nitro, amino, alkylamino, dialkylamino, loweralkoxyamino, arylaminocarbonyl, mercapto, alkylthio, sulfoxide, sulfoxide amino, sulfone, acylamino, amidino, alkenyl, cycloalkyl, phenyl, substituted phenyl, benzyl, substituted benzyl, heteroaryl, phenoxy, benzenesulfonyl, benzyloxy, or heteroaryloxy. When the parent is a heterocycle that allows such substitution, the term also includes oxides, for example pyridine-N-oxide, thiopyran sulfoxide and thiopyran-S,S-dioxide. As mentioned above, two hydrogens on a single carbon may be replaced by a carbonyl to form an oxo derivative. Noteworthy oxo-substituted aryl residues include tetralone (3,4-dihydronaphthalen-1(2H)-one) and indanone (2,3-dihydroinden-1-one).

The terms “halogen” and “halo” refer to fluorine, chlorine, bromine or iodine.

Some of the compounds described herein may contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, as well as, their racemic and optically pure forms. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration; thus a carbon-carbon double bond depicted arbitrarily herein as trans may be Z, E or a mixture of the two in any proportion.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration.

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, chlorine and iodine include ³H, ¹⁴C, ³⁵S, ¹⁸F, ³⁶Cl and ¹²⁵I, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Radiolabeled compounds of this invention can generally be prepared by methods well known to those skilled in the art. Conveniently, such radiolabeled compounds can be prepared by carrying out the procedures disclosed in the Examples by substituting a readily available radiolabeled reagent for a non-radiolabeled reagent. Because of the high affinity for the JAK3 enzyme active site, radiolabeled compounds of the invention are useful for JAK3 assays.

In one embodiment, R₄ is a heterocycle. Heterocycles that appear in the examples are monocyclic and bicyclic heterocycles or monocyclic and bicyclic heterocycles substituted with one or two substitutions. When y is not zero, heteroaryl is a preferred subset of heterocyclyl for R₄. Exemplary nitrogenous heterocycles include piperidine, pyridine, pyrazine, pyrimidine, pyridine, quinoline, isoquinoline, tetrahydroquinoline, tetrahydroisoquinoline, and their variously substituted derivatives, such as

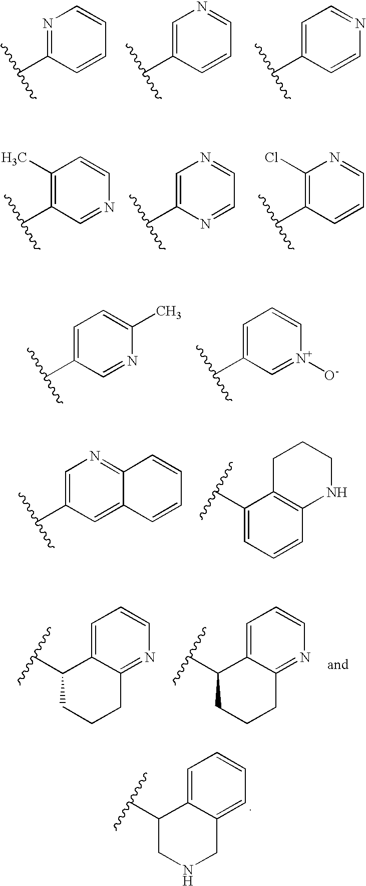

An oxygenous heterocycle is a heterocycle containing at least one oxygen in the ring; it may contain additional oxygens, as well as other heteroatoms. Exemplary oxygenous heterocycles include tetrahydropyran, chroman and their variously substituted derivatives, such as:

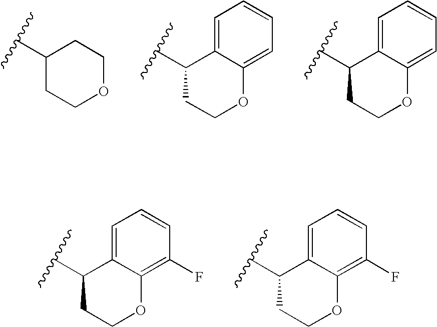

### Chemical Synthesis

Terminology related to “protecting”, “deprotecting” and “protected” functionalities occurs throughout this application. Such terminology is well understood by persons of skill in the art and is used in the context of processes that involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by T. W. Greene [John Wiley & Sons, New York, 1991], which is incorporated herein by reference.

A comprehensive list of abbreviations utilized by organic chemists appears in the first issue of each volume of the Journal of Organic Chemistry. The list, which is typically presented in a table entitled “Standard List of Abbreviations”, is incorporated herein by reference.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here. The starting materials, for example in the case of suitably substituted benzimidazole ring compounds, are either commercially available, synthesized as described in the examples or may be obtained by the methods well known to persons of skill in the art

The present invention further provides pharmaceutical compositions comprising as active agents, the compounds described herein.

As used herein a “pharmaceutical composition” refers to a preparation of one or more of the compounds described herein, or physiologically acceptable salts or solvents thereof, with other chemical components such as physiologically suitable carriers and excipients.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

Compounds that inhibit Jak-3 can be formulated as pharmaceutical compositions and administered to a mammalian subject, such as a human patient in a variety of forms adapted to the chosen route of administration, i.e., orally or parenterally, by intravenous, intramuscular, topical, transdermal or subcutaneous routes.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar or alginic acid or a salt thereof such as sodium alginate.

In addition, enteric coating may be useful as it is may be desirable to prevent exposure of the compounds of the invention to the gastric environment.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For injection, the compounds of the invention may be formulated in aqueous solutions, preferably in physiologically compatible buffers such as Hank's or Ringer's solution or physiological saline buffer. For transmucosal and transdermal administration, penetrants appropriate to the barrier to be permeated may be used in the composition. Such penetrants, including for example DMSO or polyethylene glycol, are known in the art.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions.

The compounds of the present invention may also be formulated in rectal compositions such as suppositories or retention enemas, using, e.g., conventional suppository bases such as cocoa butter or other glycerides.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on many factors including the subject being treated, the severity of the affliction, the manner of administration, the judgment of the prescribing physician. The compounds of the invention may be administered orally or via injection at a dose from 0.001 to 2500 mg/kg per day. The dose range for adult humans is generally from 0.005 mg to 10 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg. The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity.

As used herein, and as would be understood by the person of skill in the art, the recitation of “a compound” is intended to include salts, solvates and inclusion complexes of that compound. The term “solvate” refers to a compound of Formula I or II in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions. Inclusion complexes are described in Remington: The Science and Practice of Pharmacy 19th Ed. (1995) volume 1, page 176-177, which is incorporated herein by reference. The most commonly employed inclusion complexes are those with cyclodextrins, and all cyclodextrin complexes, natural and synthetic, are specifically encompassed within the claims.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

The term “preventing” as used herein refers to administering a medicament beforehand to forestall or obtund an attack. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended herein.

It should be understood that in addition to the ingredients particularly mentioned above, the formulations of this invention may include other agents conventional in the art having regard to the type of formulation in question, for example those suitable for oral administration may include flavoring agents.

The compositions may be presented in a packaging device or dispenser, which may contain one or more unit dosage forms containing the active ingredient. Examples of a packaging device include metal or plastic foil, such as a blister pack and a nebulizer for inhalation. The packaging device or dispenser may be accompanied by instructions for administration. Compositions comprising a compound of the present invention formulated in a compatible pharmaceutical carrier may also be placed in an appropriate container and labeled for treatment of an indicated condition.

### Indications

The compounds of the present invention are useful in inhibiting the activity if Jak3 or in inhibiting Jak3 mediated activity and are useful as immunosuppressive agents for tissue and organ transplants, including bone marrow transplant and in the treatment of autoimmune and inflammatory diseases and of complications arising therefrom.

Hyperacute, acute and chronic organ transplant rejection may be treated. Hyperacute rejection occurs within minutes of transplantation. Acute rejection generally occurs within six to twelve months of the transplant. Hyperacute and acute rejections are typically reversible where treated with immunosuppressant agents. Chronic rejection, characterized by gradual loss of organ function, is an ongoing concern for transplant recipients because it can occur anytime after transplantation.

There are about 75 different autoimmune disorders known that may be classified into two types, organ-specific (directed mainly at one organ) and non-organ-specific (affecting multiple organs).

Examples of organ-specific autoimmune disorders are insulin-dependent diabetes (Type I) which affects the pancreas, Hashimoto's thyroiditis and Graves' disease which affect the thyroid gland, pernicious anemia which affects the stomach, Cushing's disease and Addison's disease which affect the adrenal glands, chronic active hepatitis which affects the liver; polycystic ovary syndrome (PCOS), celiac disease, psoriasis, inflammatory bowel disease (IBD) and ankylosing spondylitis.

Examples of non-organ-specific autoimmune disorders are rheumatoid arthritis, multiple sclerosis, systemic lupus and myasthenia gravis.

Type I diabetes ensues from the selective aggression of autoreactive T-cells against insulin secreting β cells of the islets of Langerhans. Targeting Jak3 in this disease is based on the observation that multiple cytokines that signal through the Jak pathway are known to participate in the T-cell mediated autoimmune destruction of β cells. Indeed, a Jak3 inhibitor, JANEX-1 was shown to prevent spontaneous autoimmune diabetes development in the NOD mouse model of type I diabetes.

Graft-versus-host disease (GVHD) is a donor T-cell initiated pathological condition that frequently follows allogeneic bone marrow transplantation (BMT). Substantial experimental and clinical research have demonstrated that donor T-cells are the principal mediators and effectors of GVHD. Jak3 plays a key role in the induction of GVHD and treatment with a Jak3 inhibitor, JANEX-1, was shown to attenuate the severity of GVHD (reviewed in Cetkovic-Cvrlje and Ucken, 2004).

Mast cells express Jak3 and Jak3 is a key regulator of the IgE mediated mast cell responses including the release of inflammatory mediators. Jak3 was shown to be a valid target in the treatment of mast cell mediated allergic reaction.

Allergic disorders associated with mast cell activation include Type I immediate hypersensitivity reactions such as allergic rhinitis (hay fever), allergic urticaria (hives), angioedema, allergic asthma and anaphylaxis, i.e., “anaphylatic shock.” These disorders are treated or prevented by inhibition of Jak3 activity, for example, by administration of a Jak3 inhibitor according to the present invention.

According to the present invention, the Jak3 inhibitors may be administered prophylactically, i.e., prior to onset of acute allergic reaction, or they may be administered after onset of the reaction, or at both times.

Inflammation of tissues and organs occurs in a wide range of disorders and diseases and in certain variations, results from activation of the cytokine family of receptors. Exemplary inflammatory disorders associated with activation of Jak3 include, in a non-limiting manner, skin inflammation due radiation exposure, asthma, allergic inflammation and chronic inflammation.

The Jak3 inhibitors of the present invention are also useful in treating certain malignancies, including skin cancer and hematological malignancy such as lymphomas and leukemias.

The following examples will further describe the invention, and are used for the purposes of illustration only, and should not be considered as limiting the invention being disclosed.

### EXAMPLES

The following abbreviations and terms have the indicated meaning throughout:

Ac=acetyl

Bu=butyl

DCM=dichloromethane=methylene chloride=CH₂Cl₂

DEAD=diethyl azodicarboxylate

DIC=diisopropylcarbodiimide

DIEA=N,N-diisopropylethyl amine

DMF=N,N-dimethylformamide

DMSO=dimethyl sulfoxide

EA (EtOAc)=Ethyl Acetate

GC=gas chromatography

h=hours

HOAc=acetic acid

HOBt=hydroxybenzotriazole

Me=methyl

Pd(dppf)₂Cl₂=dichloro[1,1′-bis(diphenylphosphinoferrocene]palladium

Ph=phenyl

PhOH=phenol

RT=room temperature

sat'd=saturated

s-=secondary

t-=tertiary

TBDMS=t-butyldimethylsilyl

TFA=trifluoroacetic acid

THF=tetrahydrofuran

TMOF=trimethyl orthoformate

TMS=trimethylsilyl

tosyl=p-toluenesulfonyl

Trt=triphenylmethyl

Examples 1-15 describe syntheses of certain precursors and intermediates of the invention.

### Example 1

### Synthesis of 3,4-Diaminobenzonitrile

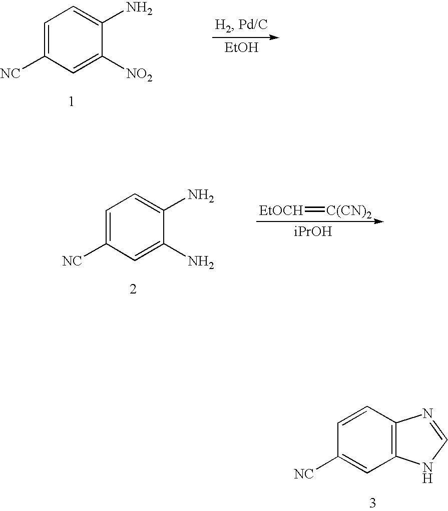

A solution of 4-amino-3-nitrobenzonitrile (1) (3.0 g) in ethanol (80 mL) was sparged for 5 minutes with nitrogen. Palladium on carbon (10%, 300 mg) was added and the mixture was saturated with hydrogen. The mixture was stirred under a hydrogen balloon for seven hours. The mixture was sparged with nitrogen and filtered through celite. The filtrate was concentrated in vacuo to provide the title compound, 3,4-diaminobenzonitrile (2).

### Example 2

### Synthesis of 3H-Benzo[d]imidazole-5-carbonitrile

A mixture of 3,4-diaminobenzonitrile (2) (1.0 g) and (ethoxymethylene)malononitrile (1.4 g) was refluxed in 50 mL of isopropyl alcohol for 16 h. The mixture was concentrated in vacuo to provide the title compound, 3H-benzo[d]imidazole-5-carbonitrile (3).

### Example 3

### Synthesis of 6-(Trifluoromethoxy)-1H-benzo[d]imidazole

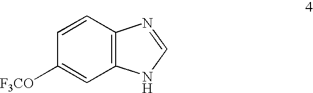

6-(trifluoromethoxy)-1H-benzo[d]imidazole (4) was prepared in two steps from 2-nitro-4-(trifluoromethoxy)aniline (5) using procedures identical to those used to make 3H-benzo[d]imidazole-5-carbonitrile (3) from 4-amino-3-nitrobenzonitrile (1, examples 1, 2).

### Example 4

### Synthesis of 5,6-Difluoro-1H-benzo[d]imidazole

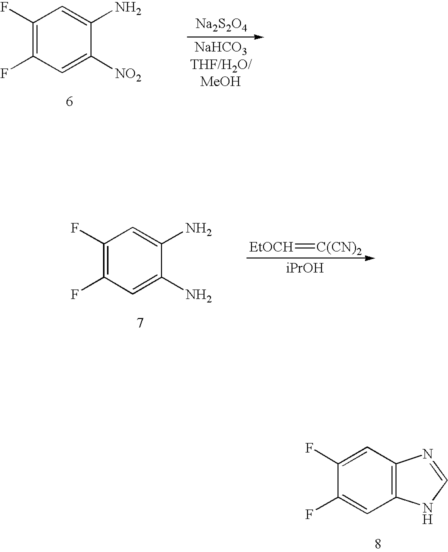

A solution of 4,5-difluoro-2-nitroaniline (6)(1.0 g) in 30 mL of THF was treated with a solution comprised of 6 g of Na₂S₂O₄ and 3 g NaHCO₃ in 30 mL of water. Methanol (10 mL) was added after the addition of the aqueous solution so that the mixture remained homogeneous. The mixture was stirred for two hours and then diluted with 100 mL of ethyl acetate and 100 mL of water. The organic layer was separated and the aqueous layer was extracted again with 100 mL of methylene chloride. The combined organic layers were dried over sodium sulfate, filtered, and concentrated to provide the crude intermediate 4,5-difluorobenzene-1,2-diamine (7). The intermediate was refluxed with (ethoxymethylene)malononitrile (1.1 g) in 25 mL of isopropyl alcohol for 16 h. The mixture was concentrated in vacuo and the resulting crude product was suspended in water and filtered. The precipitate was washed with water and air-dried to provide 380 mg of 5,6-difluoro-1H-benzo[d]imidazole (8).

### Example 5

### Synthesis of 5,6-Dimethoxy-1H-benzo[d]imidazole

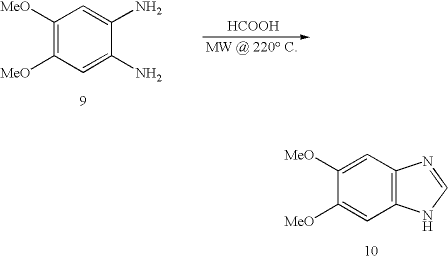

The title compound 5,6-dimethoxy-1H-benzo[d]imidazole (10) was made by heating 4,5-dimethoxy-1,2-phenylenediamine dihydrochloride (9) in formic acid at 220° C. in a microwave followed by concentration in vacuo.

### Example 6

### Synthesis of 6-Fluoro-1H-benzo[d]imidazole (11) and 6-(trifluoromethyl)-1H-benzo[d]imidazole (12)

The title compounds were by made as described in US Application Publication No. 2004/0087601, of some of the present inventors.

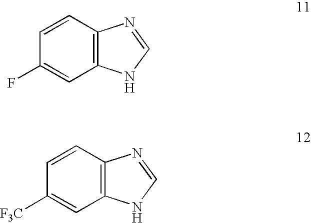

### Example 7

### Benzimidazole (13), 5-azabenzimidazole (14), 6-chloro-5-fluorobenzimidazole (15), and 5-methylbenzimidazole (16)

The title compounds were commercially available.

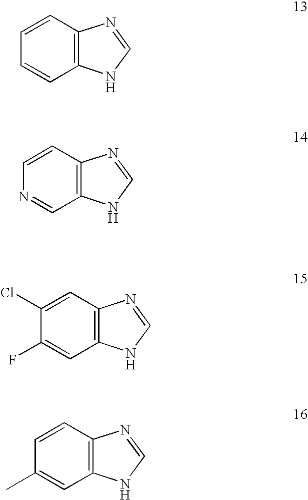

### Example 8

### Synthesis of the primary amine, pyrazin-2-ylmethanamine

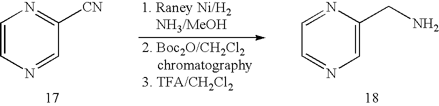

Raney nickel catalyst was carefully washed with THF and methanol making sure that the catalyst remained moist. The weight of the moist catalyst was 2.5 g after washing. This material was added to a solution of pyrazinecarbonitrile (17) (3.0 g) in 7N methanolic ammonia (120 mL). The mixture was shaken under a 50 p.s.i. atmosphere of hydrogen for 1.5 hours. The mixture was filtered and the filtrate was concentrated in vacuo to provide the crude title compound. Purification was accomplished by conversion of the crude amine to the tert-butyl carbamate with excess di-tert-butyl dicarbonate in methylene chloride. Column chromatography (70:27:3 hexanes:ethyl acetate:methanol) provided 0.50 g of pure tert-butyl pyrazin-2-ylmethylcarbamate. Pure pyrazin-2-ylmethanamine (18) was obtained as the TFA salt from deprotection of the carbamate with 1:1 TFA/CH₂Cl₂.

### Example 9

### Synthesis of 3-Aminomethyl-2-fluoropyridine

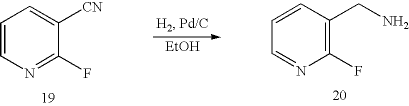

A round bottom flask was charged with 0.3 g (2.46 mM) of 3-cyano-2-fluoropyridine (19), which was then diluted in 20 mL EtOH. The solution was flushed with argon, and then while under a blanket of argon, 60 mg of 10% Pd/C (20% by weight), was added. The system was then sealed by septum and put under vacuum. A hydrogen balloon was then added, and the reaction was stirred for three hours (followed by TLC). The reaction was then put under vacuum again, then exposed to air, and filtered (keeping catalyst wet). The resulting solution was dried and evaporated to give 0.28 g (90%) of the title compound, 3-aminomethyl-2-fluoropyridine (20).

### Example 10

### Synthesis of 3-Aminomethyl-6-methoxypyridine (21), 3-Aminomethyl-6-methylpyridine (22), and 3-Aminomethylquinoline (23)

The title amines were obtained from the corresponding nitriles using the same procedure that was used to obtain 3-aminomethyl-2-fluoropyridine (20) from 3-cyano-2-fluoropyridine (see Example 9).

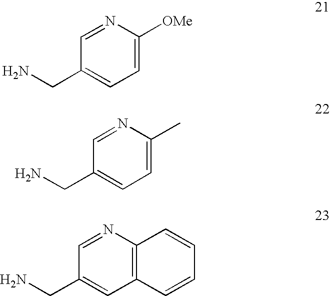

### Example 11

### Synthesis of 3-aminomethyl-2-methoxypyridine

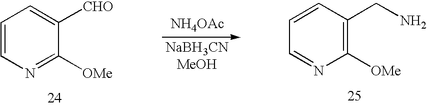

A round bottom flask was charged with 0.44 g (3.23 mM) of 2-methoxy-3-pyridine carboxaldehyde (24), 1.24 g (16.15 mM) of ammonium acetate, and 0.61 g (19.69 mM) of sodium cyanoborohydride. The flask was then flushed with argon, and then 50 mL of dry MeOH was added by syringe. The reaction was stirred for 2 days, at which point the MeOH was evaporated off. 25 mL of water was added, and the mixture was brought to pH 2 with conc. HCl. This was extracted twice with EtOAc to remove the alcohol side product. The mixture was brought to pH 10 using sodium hydroxide pellets, saturated with NaCl, and extracted twice with DCM and once with EtOAc. The combined organics were dried and evaporated to give 0.31 g (69%) of 3-aminomethyl-2-methoxypyridine (25).

### Example 12

### Synthesis of 3-α-aminoethyl)-2-chloropyridine (26)

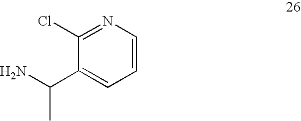

The title amine was obtained from the corresponding ketone using the same procedure that was used to obtain 3-aminomethyl-2-methoxypyridine from 2-methoxy-3-pyridine carboxaldehyde (24; Example 11).

### Example 13

### Synthesis of 3-aminomethyl-4-methylpyridine

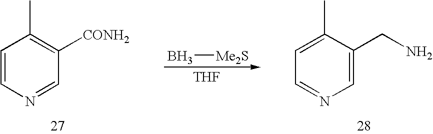

A round bottom flask was charged with 0.45 g (3.30 mM) of 4-methylnicotinamide (27). The flask was flushed with argon, and 50 mL of dry THF was added by syringe. The resulting solution was cooled to 0 dg C., and 2.5 mL (4.96 mM) of a 2M solution of borane-dimethylsulfide complex (in THF) was added. A bubbler was attached, and the solution was allowed to warm to RT overnight. The solution was quenched with MeOH, and dried and evaporated to give 0.38 g (95%) of 3-aminomethyl-4-methylpyridine (28).

### Example 14

### Synthesis of 5-fluoro-1,2,3,4-tetrahydronaphthalen-1-amine

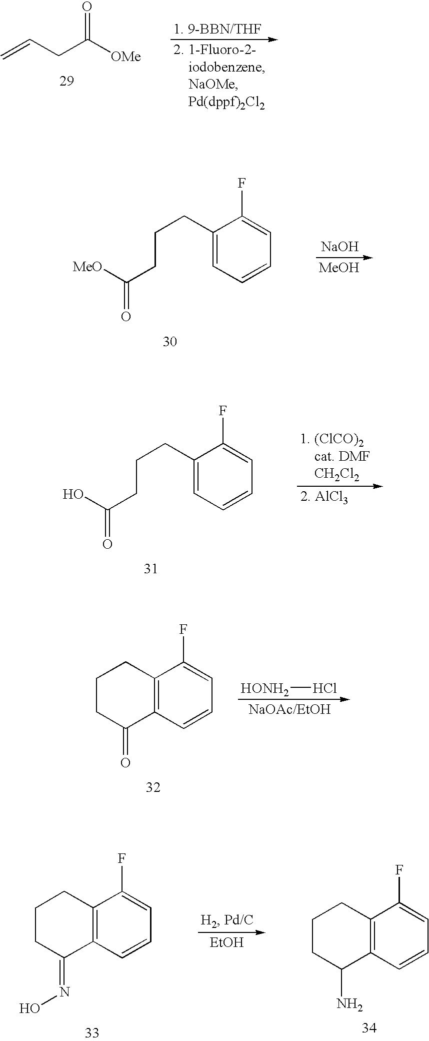

Methyl 4-(2-fluorophenyl)butanoate (2-(4-methylbutanoate)fluorobenzene, 30). A round bottom flask was sealed with a rubber septum, flushed with argon, then charged with 5.32 mL of methyl 3-buteneoate (29) and 100 mL of a 0.5M solution of 9-BBN in THF. The solution was stirred at RT for three hours. A 2-necked round bottom flask was equipped with a condenser and flushed with argon, then charged with 7.36 g of sodium methoxide and 1.11 g of Pd(dppf)₂Cl₂. To this mixture were added 20 mL of dry THF and 5.22 mL of 1-fluoro-2-iodobenzene. The hydroboration solution was added via canula and the resulting mixture was refluxed for 16 hours. The solution was cooled to RT, diluted with 150 mL of water, and extracted three times with ether. The combined organic layers were washed with brine, dried, and evaporated. Column chromatography (5% EtOAc/hexanes) gave 1.79 g methyl 4-(2-fluorophenyl)butanoate (30).

4-(2-Fluorophenyl)butanoic acid (31). A round bottom flask was charged with 1.79 g of 2-(4-methylbutanoate)fluorobenzene, which was dissolved in 17 mL of MeOH. To this solution was added a solution of 1 g of sodium hydroxide. The resulting mixture was stirred 20 hours at RT. The solvent was evaporated and the crude material diluted with 15 mL of 0.5M HCl. Extraction with DCM three times gave 1.17 g (92%) of 4-(2-fluorophenyl)butanoic acid (31).

5-Fluoro-3,4-dihydronaphthalen-1(2H)-one (32). A round bottom flask was charged with 0.15 g of 4-(2-fluorophenyl)butanoic acid, which was dissolved in 20 mL DCM and cooled to 0° C. Oxalyl chloride (0.15 mL) was added, followed by 1 drop of DMF. A drying tube was attached, and the solution was stirred at 0° C. for two hours. Aluminum chloride (0.121 g) was added and the solution was allowed to slowly warm to RT overnight. The mixture was poured onto ice water, and extracted three times with DCM. The combined organic layers were washed with 0.5 M NaOH and brine. The organic phase was dried, evaporated, and purified by column chromatography (eluting with 20% EtOAc/Hexanes), to give 0.07 g (53%) of 5-fluoro-3,4-dihydronaphthalen-1(2H)-one (32).

5-Fluoro-1,2,3,4-tetrahydronaphthalen-1-amine (34). A round bottom flask was charged with 0.5 g of 5-fluoro-3,4-dihydronaphthalen-1(2H)-one, 0.28 g of hydroxylamine hydrochloride, and 0.34 g of sodium acetate. A condenser was attached, and the flask was purged with argon. 20 mL of dry EtOH was added, and the mixture was stirred at reflux for 18 hours. The solution was cooled to RT, diluted with EtOAc, and washed with water. The organic phase was dried with sodium sulfate and evaporated to give 0.5 g of the intermediate 5-fluoro-3,4-dihydronaphthalen-1(2H)-one oxime (33), which was reduced with Pd/C in EtOH with hydrogen (50 psi), to give 0.43 g (86%) of 5-fluoro-1,2,3,4-tetrahydronaphthalen-1-amine (34).

### Example 15

### Synthesis of 8-fluorochroman-4-amine

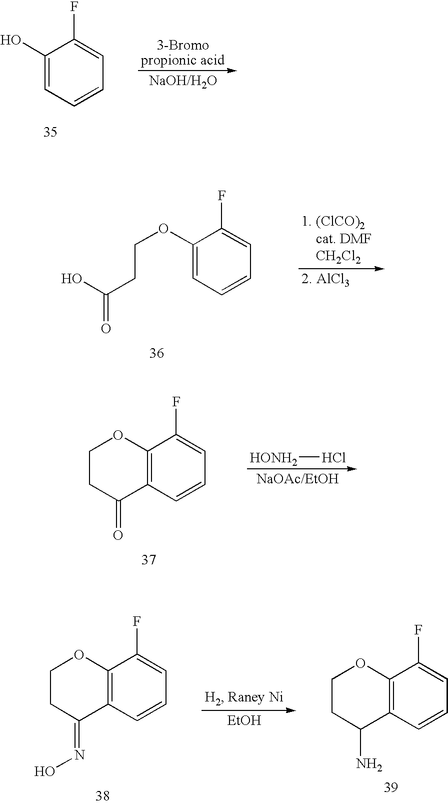

3-(2-fluorophenoxy)propanoic acid (36). A mixture of 2-fluorophenol (35) (15 g), 3-bromopropanoic acid (20 g) and NaOH (11 g) was refluxed in 50 mL of water. The solution was cooled to RT and acidified to pH 2 with 3 M HCl. The resulting precipitate was isolated by filtration to yield 9.27 g of title compound as a white solid. The filtrate was extracted three times with EtOAc to yield 2.5 g of less pure compound (36).

8-fluorochroman-4-one (37). Oxalyl chloride (8.79 mL) and one drop of DMF were added to an ice cold solution of 3-(2-fluorophenoxy)propanoic acid (9.27 g) in DCM (50 mL). The solution was stirred at 0° C. for two hours, aluminum chloride (7.39 g, 55.42 mM) was added and the solution was stirred for 16 hours at RT. The mixture was poured onto ice water, and extracted three times with DCM. The combined organics were washed with 0.5M NaOH and brine, dried, evaporated, and purified by column chromatography (eluting with 20% EtOAc/Hex) to give 8-fluorochroman-4-one (37) (8.20 g, 98%).

8-fluorochroman-4-amine (39). A round bottom flask was charged with 8-fluorochroman-4-one (8.2 g), hydroxylamine hydrochloride (3.78 g) and sodium acetate (4.46 g). A reflux condenser was added, the flask was purged with argon, dry EtOH (20 mL) was added, and the mixture was stirred at reflux for 18 hours. The solution was cooled to RT, diluted with EtOAc, and washed with water. The organic phase was dried, and evaporated to give the intermediate 8-fluorochroman-4-one oxime (38), which was reduced with Raney Nickel in EtOH at 50 PSI to yield the titled amine (39) (4.69 g, 57%).

Resolution of 8-fluorochroman-4-amine.

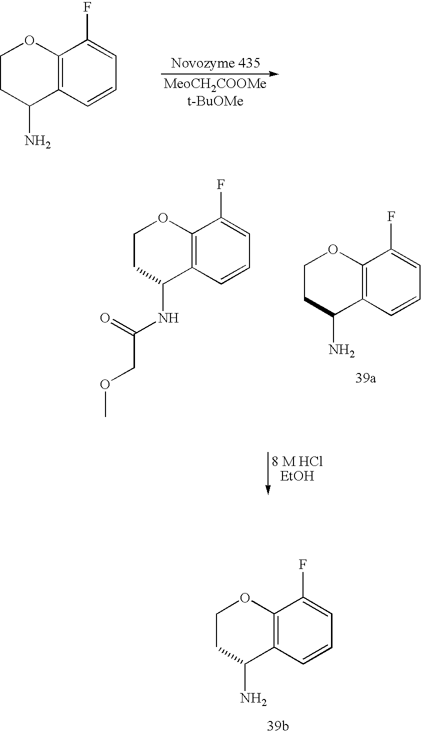

In short, a mixture of 8-fluorochroman-4-amine (3.40 g), methyl 2-methoxyacetate (2.44 g) and Novozyme 435 (Aldrich, 0.68 g) in anhydrous tert-butyl methyl ether (75 mL) was heated at reflux under argon for two hours (at which time the ratio of acylated to unacylated product was 1:1 by HPLC). The solid that formed upon cooling was collected via filtration and dissolved in EtOAc. The mixture was filtered to remove the biocatalyst and washed once with 0.5M HCl to remove any lingering (S)-amine. The solvent was evaporated and the product was recrystallized from tert-butyl methyl ether to yield (R)—N-(8-fluorochroman-4-yl)-2-methoxyacetamide (0.78 g). The reaction solvent and recrystallization mother liquor was washed three times with 0.5 M HCl and concentrated to yield additional (R)—N-(8-fluorochroman-4-yl)-2-methoxyacetamide (0.83 g). The combined acidic aqueous layers were made basic by NaOH and extracted with DCM to yield (S)-8-fluorochroman-4-amine (39a) (1.6 g). A solution of (R)—N-(8-fluorochroman-4-yl)-2-methoxyacetamide (0.78 g) in 8M HCl in EtOH (50 mL) was heated at reflux for four hours. The solvents were removed from the cooled reaction mixture, the resulting solid was taken up in 50 mL of 0.5M NaOH, salted out with NaCl₍ₛ₎, and extracted four times with DCM to yield (R)-8-fluorochroman-4-amine (0.48 g (87%))(39b). The % ee was checked via chiral HPLC: Chiralcel OD-H (0.46×25 cm analytical column, Daicel Chemical Industries) method: isocratic 5% (0.05% TFA/EtOH) 95% (0.05% TFA/Hex), Rt=7.2 min (S)-enantiomer, Rt=9.2 min (R)-enantiomer.

### Example 16

### Synthesis of 2-(1H-Benzo[d]imidazol-1-yl)-9-((R)-8-fluorochroman-4-yl)-7H-purin-8(9H)-one

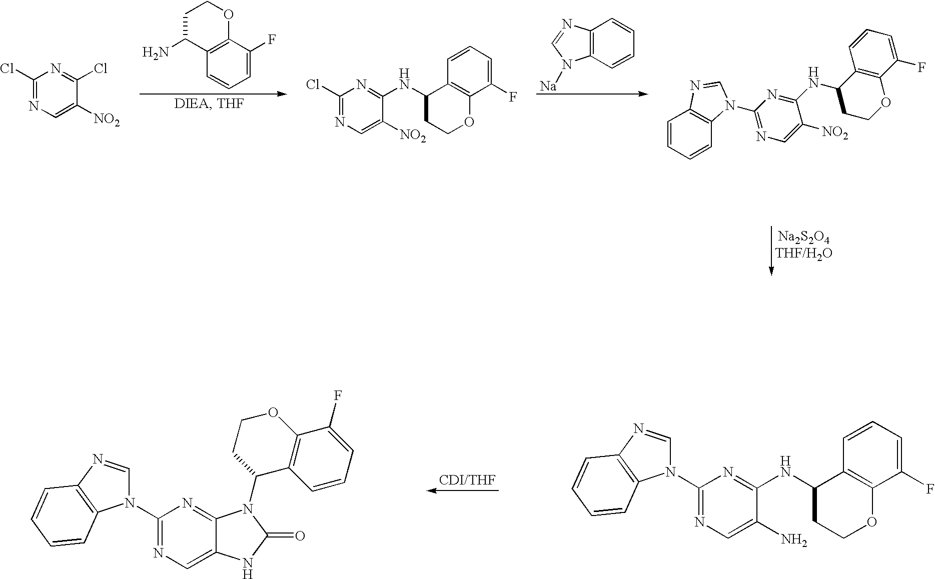

2-(1H-Benzo[d]imidazol-1-yl)-N—((R)-8-fluorochroman-4-yl)-5-nitropyrimidin-4-amine. (R)-8-fluorochroman-4-amine (60 mg, Example 15) was added to a solution of 2,4-dichloro-5-nitropyrimidine (70 mg) and DIEA (0.14 mL) in THF (5 mL) at −78° C. The reaction mixture was stirred for a further 15 min at −78° C. then removed from the cold bath and allowed to warm to RT. A one molar solution of the sodium salt of benzimidazole (0.7 ml, stock solution prepared via the addition of sodium hydride to a benzimidazole solution in THF) was added to the reaction intermediate ((R)-2-chloro-N-(8-fluorochroman-4-yl)-5-nitropyrimidin-4-amine) and the resulting mixture was stirred at RT overnight. Purification via column chromatography (elution with 1 MeOH/DCM) gave the titled compound (120 mg), MH⁺=407.

2-(1H-Benzo[d]imidazol-1-yl)-9-((R)-8-fluorochroman-4-yl)-7H-purin-8(9H)-one. A freshly prepared solution of sodium hydrosulfite (tech, 0.5 g) and sodium bicarbonate (0.25 g) in H₂O (5 mL) was added to a solution of the above nitro compound (120 mg) in THF (10 mL). The mixture was stirred vigorously for 30 min then extracted with EtOAc (2×) and DCM (2×), the combined organics were washed with brine, dried, filtered and concentrated to yield the intermediate 2-(1H-benzo[d]imidazol-1-yl)-N⁴—((R)-8-fluorochroman-4-yl)pyrimidine-4,5-diamine that was used as such in the next step.

Carbonyldiimidazole (0.2 g) was added to a solution of the above amine in THF (10 mL). The resulting mixture was stirred at RT overnight, silica gel was added, then the solvents were removed under reduced pressure and purified via column chromatography, elution with 5% MeOH/DCM, to yield the titled product (28 mg), MH⁺=403, ¹H NMR (CDCl₃) δ 10.6 (s, 1H), 8.9 (s, 1H), 8.3 (s, 1H), 7.8 (m, 2H), 7.3 (m, 2H), 7.0 (m, 1H), 6.7 (m, 1H), 5.9 (dd, 1H), 4.7 (m, 1H), 4.4 (t, 1H), 2.7 (m, 1H), 2.3 (m, 1H) ppm, ¹⁹F NMR 6-135.7 (m). Chiral HPLC—no evidence of other enantiomer, Method; Chiralcel OD-H (0.46×25 cm analytical column, Daicel Chemical Industries) isocratic 15% (0.05% TFA/EtOH) 85% (0.05% TFA/Hex), Rt=19.5 min (R)-enantiomer, Rt=22.4 min (S)-enantiomer.

### Example 17

### Non-regiospecific synthesis of benzimidazole purinone derivatives: Synthesis of 5-Nitro-N-(pyridin-3-ylmethyl)-2-(6-(trifluoromethoxy)-1H-benzo[d]imidazol-1-yl)pyrimidin-4-amine (42) AND 5-nitro-N-(pyridin-3-ylmethyl)-2-(5-(trifluoromethoxy)-1H-benzo[d]imidazol-1-yl)pyrimidin-4-amine (44)

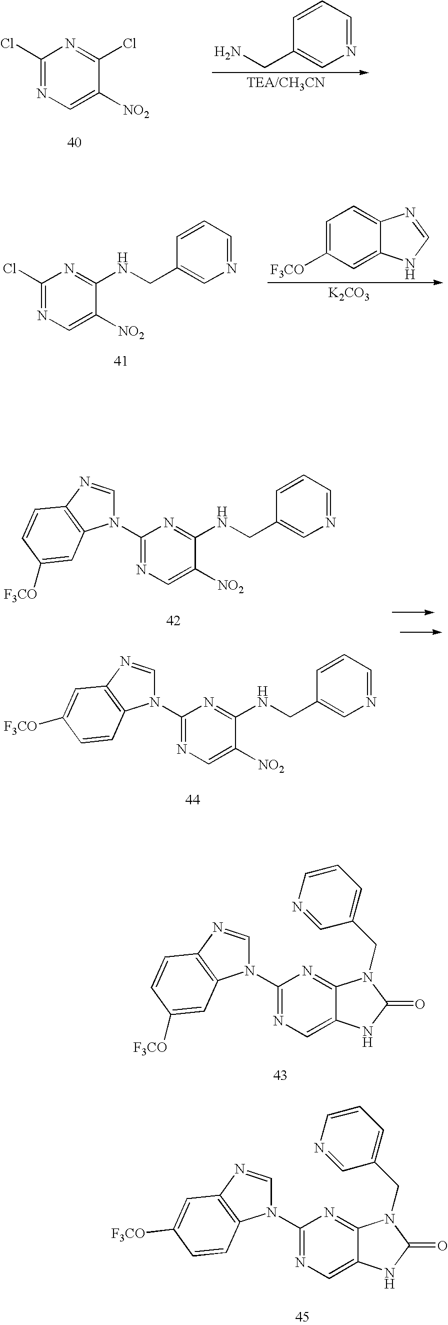

2-Chloro-5-nitro-N-(pyridin-3-ylmethyl)pyrimidin-4-amine (41). A solution of 2,4-dichloro-5-nitropyrimidine (40) (5 g) in methylene chloride (60 mL) was cooled to −78° C. and treated with 3-(aminomethyl)pyridine (2.8 g). The mixture was stirred at −78° C. for six hours, and concentrated in vacuo at RT to provide crude 2-chloro-5-nitro-N-(pyridin-3-ylmethyl)pyrimidin-4-amine (41), which was used without further purification.

5-Nitro-N-(pyridin-3-ylmethyl)-2-(6-(trifluoromethoxy)-1H-benzo[d]imidazol-1-yl)pyrimidin-4-amine (42) and 5-nitro-N-(pyridin-3-ylmethyl)-2-(5-(trifluoromethoxy)-1H-benzo[d]imidazol-1-yl)pyrimidin-4-amine (44). A suspension of crude 2-chloro-5-nitro-N-(pyridin-3-ylmethyl)pyrimidin-4-amine (52 mg) in acetonitrile (10 mL) was treated with 6-(trifluoromethoxy)-1H-benzo[d]imidazole (40 mg), potassium carbonate (0.5 g), and heated at 80° C. for four hours. The mixture was diluted with water and extracted with methylene chloride. The organic layer was separated, dried with sodium sulfate, filtered, and concentrated in vacuo. Column chromatography (70:22:8 methylene chloride:ethyl acetate:methanol) provided 12 mg of 5-nitro-N-(pyridin-3-ylmethyl)-2-(5-(trifluoromethoxy)-1H-benzo[d]imidazol-1-yl)pyrimidin-4-amine as the first eluting isomer and 15 mg of 5-nitro-N-(pyridin-3-ylmethyl)-2-(6-(trifluoromethoxy)-1H-benzo[d]imidazol-1-yl)pyrimidin-4-amine as the second eluting isomer.

High R_{f} isomer: ¹H-NMR (CDCl₃) δ 9.2 (s, 1H), 8.9 (s, 1H), 8.8 (m, 1H), 8.6 (s, 1H), 8.5 (d, 1H), 8.2 (d, 1H, assign: H-7 of benzimidazole ring), 7.6 (d, 1H), 7.6 (s, 1H, assign: H-4 of benzimidazole ring), 7.2 (dd, 1H), 4.9 (d, 2H).

Low R_{f} isomer: ¹H-NMR (CDCl₃) δ 9.2 (s, 1H), 8.9 (s, 1H), 8.8 (m, 1H), 8.6 (s, 1H), 8.5 (d, 1H), 8.2 (s, 1H, assign: H-7 of benzimidazole ring), 7.7 (d, 1H, assign: H-4 of benzimidazole ring), 7.6 (d, 1H), 7.2 (dd, 1H), 7.1 (d, 1H), 4.9 (d, 2H).

### Example 18

### Non-regiospecific synthesis of benzimidazole purinone derivatives: Synthesis of 9-(Pyridin-3-ylmethyl)-2-(6-(trifluoromethoxy)-1H-benzo[d]imidazol-1-yl)-7H-purin-8(9H)-one (43) AND 9-(pyridin-3-ylmethyl)-2-(5-(trifluoromethoxy)-1H-benzo[d]imidazol-1-yl)-7H-purin-8(9H)-one (45)

The title compounds were synthesized from 5-nitro-N-(pyridin-3-ylmethyl)-2-(6-(trifluoromethoxy)-1H-benzo [d]imidazol-1-yl)pyrimidin-4-amine (42) and 5-nitro-N-(pyridin-3-ylmethyl)-2-(5-(trifluoromethoxy)-1H-benzo[d]imidazol-1-yl)pyrimidin-4-amine (44) using the same procedures that were used to convert (R)-tert-butyl 2,4-dimethoxybenzyl(5-nitro-6-(1-(pyridin-3-yl)ethylamino)pyridin-2-yl)carbamate to (R)-tert-butyl 2,4-dimethoxybenzyl(2-oxo-3-(1-(pyridin-3-yl)ethyl)-2,3-dihydro-1H-imidazo[4,5-b]pyridin-5-yl)carbamate (67; Example 22 below).

6-Trifluoromethoxy isomer (non-salt): ¹H-NMR (CD₃OD) δ 9.3 (s, 1H), 8.8 (br s, 1H), 8.6 (s, 1H, assign: H-7 of benzimdazole ring), 8.6 (m, 1H), 8.4 (s, 1H), 8.1 (d, 1H), 7.9 (d, 1H, assign: H-4 of benzimidazole ring), 7.5 (dd, 1H), 7.4 (dd, 1H), 5.4 (s, 2H).

5-Trifluoromethoxy isomer (non-salt): ¹H-NMR (CD₃OD) δ 9.3 (s, 1H), 8.8 (s, 1H), 8.7 (d, 1H, assign: H-7 of benzimdazole ring), 8.5 (d, 1H), 8.3 (s, 1H), 7.9 (d, 1H), 7.6 (s, 1H, assign: H-4 of benzimidazole ring), 7.4 (dd, 1H), 7.3 (dd, 1H), 5.3 (s, 2H).

### Example 19

### Non-regiospecific synthesis of an oxoimidazopyridine and an imidazopyridine derivative: Synthesis of 5-(1H-Benzo [d]imidazol-1-yl)-3-(pyridin-3-ylmethyl)-1H-imidazo[4,5-b]pyridin-2(3H)-one (50)

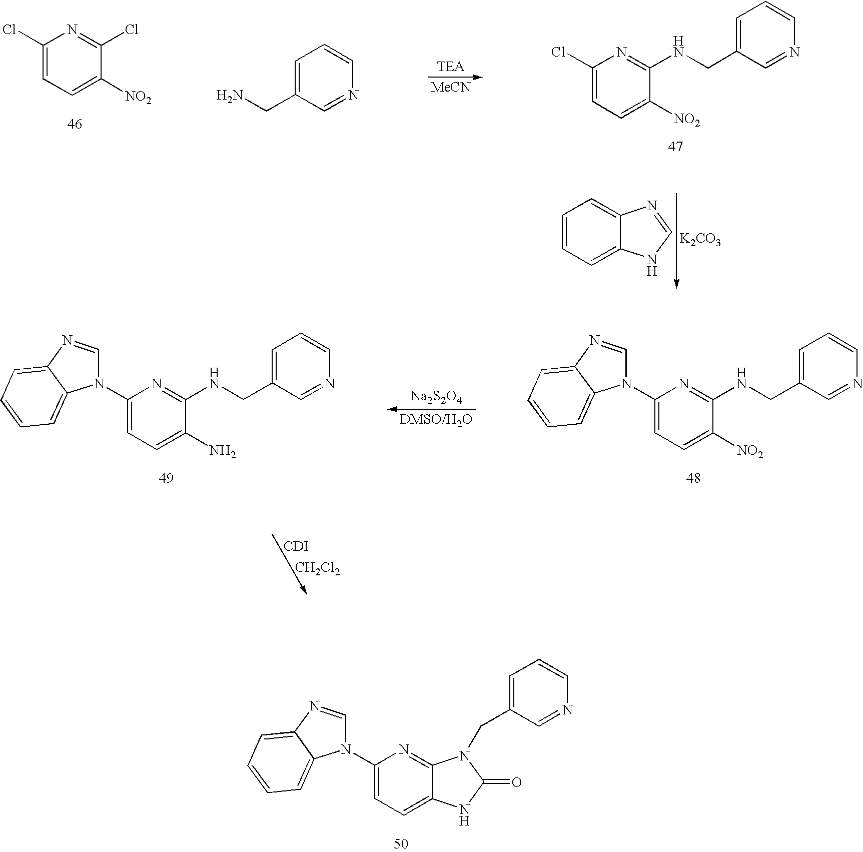

6-(1H-Benzo[d]imidazol-1-yl)-3-nitro-N-(pyridin-3-ylmethyl)pyridin-2-amine (48). A solution of 2,6-dichloro-3-nitropyridine (46) (0.5 g) in acetonitrile (20 mL) was cooled to 0° C. and treated with triethylamine (0.36 mL) followed by 3-(aminomethyl)pyridine (0.26 mL). The mixture was stirred for 30 minutes at 0° C. and eight hours at RT. The resulting solution, which contained the intermediate 6-chloro-3-nitro-N-(pyridin-3-ylmethyl)pyridin-2-amine (47), was transferred to a sealed tube containing benzimidazole (0.84 g) and potassium carbonate (3 g) and heated at 70° C. for 16 h. The mixture was cooled and filtered. The precipitated was washed with water and air-dried to provide 239 mg of the title compound (48).

5-(1H-Benzo[d]imidazol-1-yl)-3-(pyridin-3-ylmethyl)-1H-imidazo[4,5-b]pyridin-2(3H)-one (50) and 5-(1H-benzo[d]imidazol-1-yl)-3-(pyridin-3-ylmethyl)-3H-imidazo[4,5-b]pyridine (51). A solution of 6-(1H-benzo[d]imidazol-1-yl)-3-nitro-N-(pyridin-3-ylmethyl)pyridin-2-amine (48) (50 mg) in 1 mL of DMSO was treated with a solution of Na₂S₂O₄ (300 mg) in 1 mL of water. The mixture was stirred for two hours and diluted with 50 mL of ethyl acetate. The mixture was washed three times with 50 mL aliquots of saturated sodium chloride solution, dried over sodium sulfate, filtered, and concentrated in vacuo to provide the intermediate 6-(1H-benzo[d]imidazol-1-yl)-N2-(pyridin-3-ylmethyl)pyridine-2,3-diamine (49). Half of the intermediate was dissolved in methylene chloride (2 mL) and treated with 1,1′-carbonyldiimidazole (46 mg) at RT for 16 h. The resulting crude mixture was purified by preparative TLC (1000 microns, 5% MeOH/CH₂Cl₂) to provide 7.1 mg of 5-(1H-benzo[d]imidazol-1-yl)-3-(pyridin-3-ylmethyl)-1H-imidazo[4,5-b]pyridin-2(3H)-one (50): ¹H-NMR (CDCl₃) δ 10.0 (br s, 1H), 8.9 (s, 1H), 8.6 (d, 1H), 8.5 (s, 1H), 7.9 (m, 2H), 7.8 (m, 1H), 7.6 (d, 1H), 7.4 (m, 2H), 7.4 (m, 1H), 7.3 (d, 1H), 5.2 (s, 2H).

### Example 20

### Regiospecific synthesis: Synthesis of 3-(9-(2,6-Difluorobenzyl)-8-oxo-8,9-dihydro-7H-purin-2-yl)-3H-benzo[d]imidazole-5-carbonitrile

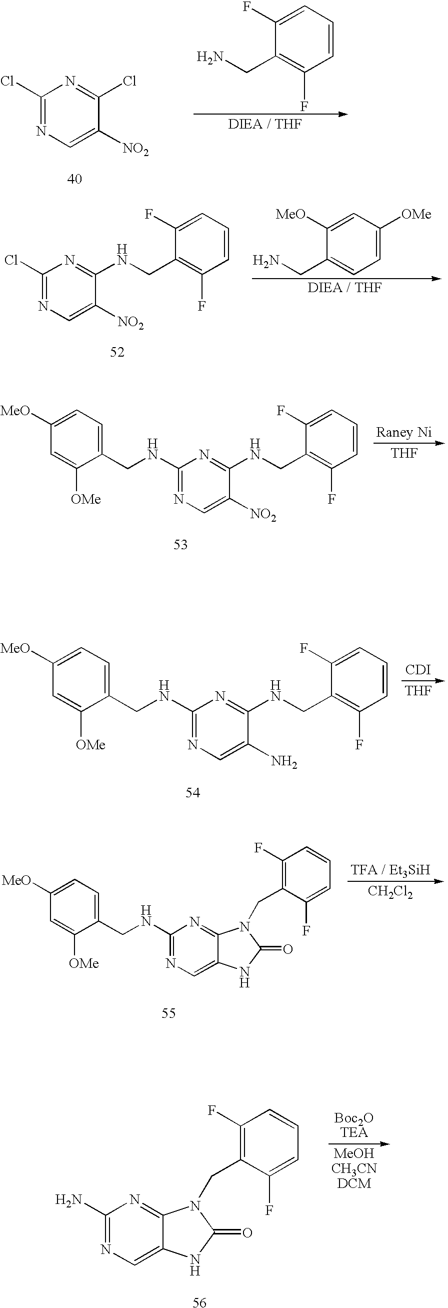

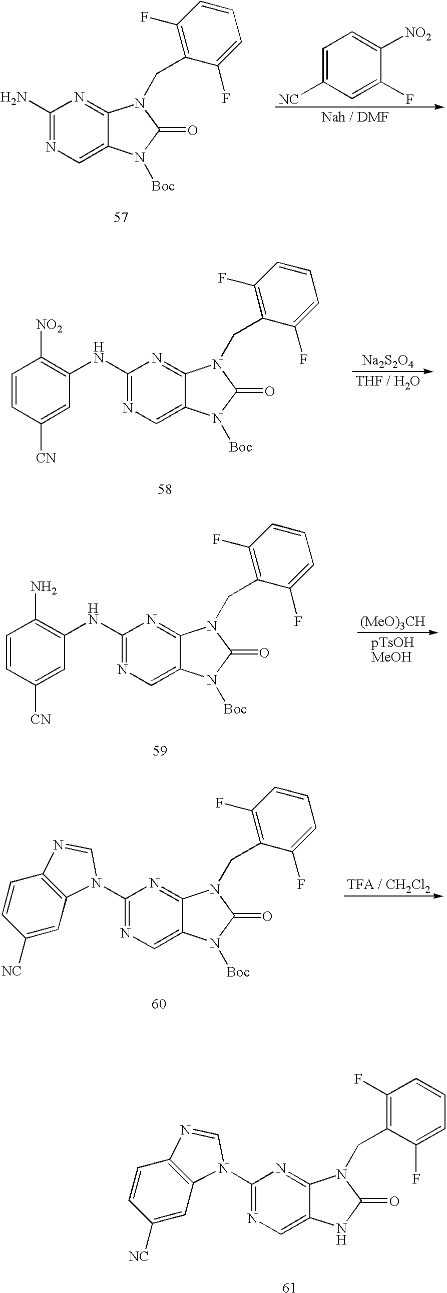

N²-(2,4-Dimethoxybenzyl)-N⁴-(2,6-difluorobenzyl)-5-nitropyrimidine-2,4-diamine (53). 2,6-Difluorobenzylamine (0.24 mL) was added dropwise over one min. to a solution of 2,4-dichloro-5-nitropyrimidine (40) (0.388 g) and DIEA (0.77 mL) in THF in a cold bath set to −78° C. The reaction mixture was stirred for a further 15 min at −78° C. then removed from the cold bath and allowed to warm to RT. Additional DIEA (0.77 mL) was added to the reaction intermediate (N-(2,6-difluorobenzyl)-2-chloro-5-nitropyrimidin-4-amine) (52) followed by the addition of 2,4-dimethoxybenzylamine (0.30 mL) and the resulting mixture was stirred at RT overnight. Purification via column chromatography (eluted with 1 and 2.5% MeOH/DCM) gave N²-(2,4-dimethoxybenzyl)-N⁴-(2,6-difluorobenzyl)-5-nitropyrimidine-2,4-diamine (53) (0.80 g), MH⁺=432.

2-(2,4-Dimethoxybenzylamino)-9-(2,6-difluorobenzyl)-7H-purin-8(9H)-one (55). Raney Ni was added to a solution of N²-(2,4-dimethoxybenzyl)-N⁴-(2,6-difluorobenzyl)-5-nitropyrimidine-2,4-diamine (0.80 g) in THF (50 mL) under argon flush. The suspension was evacuated, charged with hydrogen (balloon) and stirred for 16 hr. The resulting mixture was filtered through a celite plug that was thoroughly rinsed with THF and MeOH to yield N²-(2,4-dimethoxybenzyl)-N⁴-(2,6-difluorobenzyl)pyrimidine-2,4,5-triamine (54) that was used as such in the next reaction.

Carbonyldiimidazole (0.93 g) was added to a solution of N²-(2,4-dimethoxybenzyl)-N⁴-(2,6-difluorobenzyl)pyrimidine-2,4,5-triamine (54) in THF (20 mL) and the resultant mixture stirred at RT overnight, then the solvents were removed under reduced pressure and the taken up in EtOAc and washed trice with water. The organics were dried, filtered and evaporated and purified via column chromatography, elution with 2.5 and 4% MeOH/DCM, to yield 2-(2,4-Dimethoxybenzylamino)-9-(2,6-difluorobenzyl)-7H-purin-8(9H)-one (55) (0.58 g), MH⁺=428.

tert-Butyl 9-(2,6-difluorobenzyl)-2-amino-8-oxo-8,9-dihydropurine-7-carboxylate (57). A 1:1 solution of TFA/DCM (10 mL) was added to 2-(2,4-dimethoxybenzylamino)-9-(2,6-difluorobenzyl)-7H-purin-8(9H)-one (55) (0.58 g) and stirred for 30 min, after which triethylsilane (2 mL) was added and the mixture was stirred an additional 4 hr. The solvents were removed under in vacuo, the residue was taken up in minimal MeOH and triturated with Et₂O, to yield the TFA salt of 9-(2,6-difluorobenzyl)-2-amino-7H-purin-8(9H)-one (56) (0.55 g), MH⁺=278, as a salmon colored solid.

9-(2,6-Difluorobenzyl)-2-amino-7H-purin-8(9H)-one (0.55 g) was dissolved in a mixture of MeOH/ACN/DCM (40 mL), Et₃N (2 mL) and di-tert-butyl dicarbonate (0.61 g) were added and the mixture was stirred at RT overnight. The reaction solvents were removed and the crude material was taken up in DCM and washed with H₂O, evaporated and purified via column chromatography, elution with 2 and 3% MeOH/DCM gave the titled product (57) (0.36 g), MH⁺=378, MH⁺—Boc=278 (major), (M+Na)⁺=400 and (2M+Na)⁺=777 were also observed.

tert-Butyl 9-(2,6-difluorobenzyl)-2-(5-cyano-2-nitrophenylamino)-8-oxo-8,9-dihydropurine-7-carboxylate (58). Sodium hydride (88 mg, 95%) was added, under argon flush, to a solution of tert-butyl 9-(2,6-difluorobenzyl)-2-amino-8-oxo-8,9-dihydropurine-7-carboxylate (57) (191 mg) and 3-fluoro-4-nitrobenzonitrile (415 mg) in DMF (5 mL) at −40° C. The reaction mixture was allowed to warm to −20° C. over 3 hr then quenched by the addition of sat. aq. NH₄Cl, once at RT the mixture was diluted with EtOAc and separated. The organics were washed with brine (3×), dried, filtered and evaporated, purified via column chromatography, (eluted with DCM and 1 and 2.5% MeOH/DCM) to yield tert-Butyl 9-(2,6-difluorobenzyl)-2-(5-cyano-2-nitrophenylamino)-8-oxo-8,9-dihydropurine-7-carboxylate (58) (288 mg), MH⁺=524.

tert-Butyl 9-(2,6-difluorobenzyl)-2-(6-cyano-1H-benzo[d]imidazol-1-yl)-8-oxo-8,9-dihydropurine-7-carboxylate (60). A freshly prepared solution of sodium hydrosulfite (tech, 1 g) and sodium bicarbonate (0.5 g) in H₂O (10 mL) was added to a solution of the above nitro compound (58) (288 mg) in THF (10 mL). The mixture was stirred vigorously for 5 min., extracted with DCM (3×), the combined organics were washed with brine, dried, filtered and concentrated to yield the intermediate tert-butyl 9-(2,6-difluorobenzyl)-2-(2-amino-5-cyanophenylamino)-8-oxo-8,9-dihydropurine-7-carboxylate (59) that was used as such in the next step.

A catalytic amount of para-toluene sulfonic acid monohydrate was added to a solution of the above amine intermediate and trimethyl orthoformate (3 mL) in MeOH (10 mL). After 1 hr the crude material was adsorbed onto silica gel and purified by column chromatography (eluted with 1 and 2% MeOH/DCM) to yield tert-Butyl 9-(2,6-difluorobenzyl)-2-(6-cyano-1H-benzo[d]imidazol-1-yl)-8-oxo-8,9-dihydropurine-7-carboxylate (60) (164 mg), MH⁺=504 and MH⁺—BOC=404.

3-(9-(2,6-Difluorobenzyl)-8-oxo-8,9-dihydro-7H-purin-2-yl)-3H-benzo[d]imidazole-5-carbonitrile (61). A 1:1 solution of TFA/DCM (10 mL) was added to tert-Butyl 9-(2,6-difluorobenzyl)-2-(6-cyano-1H-benzo[d]imidazol-1-yl)-8-oxo-8,9-dihydropurine-7-carboxylate (60) and stirred for 1 hr. The solvents were removed en vacuo and the resulting solid was triturated with Et₂O, and suspended in 6N HCl. Removal of solvents and trituration with Et₂O of the resulting solid gave the titled compound (61) (68 mg) as a HCl salt, MH⁺=404, ¹H NMR (d₆-DMSO) δ 11.8 (s, 1H), 9.2 (s, 1H), 8.8 (s, 1H), 8.0 (s, 1H), 7.9 (d, 1H), 7.8 (broad s, 1H), 7.4 (d, 1H), 7.4 (quintet, 1H), 7.1 (m, 2H), 5.2 (s, 2H) ppm, ¹⁹F NMR δ−114.3 (m).

### Example 21

### Synthesis of 3-(8-Oxo-9-(tetrahydro-2H-pyran-4-yl)-8,9-dihydro-7H-purin-2-yl)-3H-benzo[d]imidazole-5-carbonitrile (62)

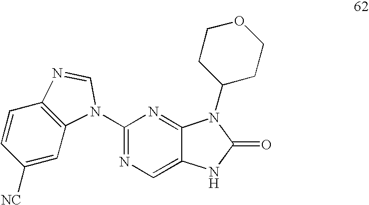

The title compound can be synthesized using the same procedures as described for the synthesis of 3-(9-(2,6-difluorobenzyl)-8-oxo-8,9-dihydro-7H-purin-2-yl)-3H-benzo[d]imidazole-5-carbonitrile (61, Example 20).

¹H NMR (d₆-DMSO) 11.71 (s, 1H), 9.34 (s, 1H), 8.94 (d, J=1.5 Hz, 1H), 8.38 (s, 1H), 8.00 (d, J=8.1 Hz, 1H), 7.79 (dd, J=8.1, 1.5 Hz, 1H), 4.57 (m, 1H), 4.04 (m, 2H), 3.50 (m, 2H), 2.59 (m, 2H), 1.79 (m, 2H); Mass (MH+) 362.1.

### Example 22

### Regiospecific synthesis of an oxoimidazopyridine derivative: Synthesis of 3-(2-oxo-3-((R)-1-(pyridin-3-yl)ethyl)-2,3-dihydro-1H-imidazo[4,5-b]pyridin-5-yl)-3H-benzo[d]imidazole-5-carbonitrile

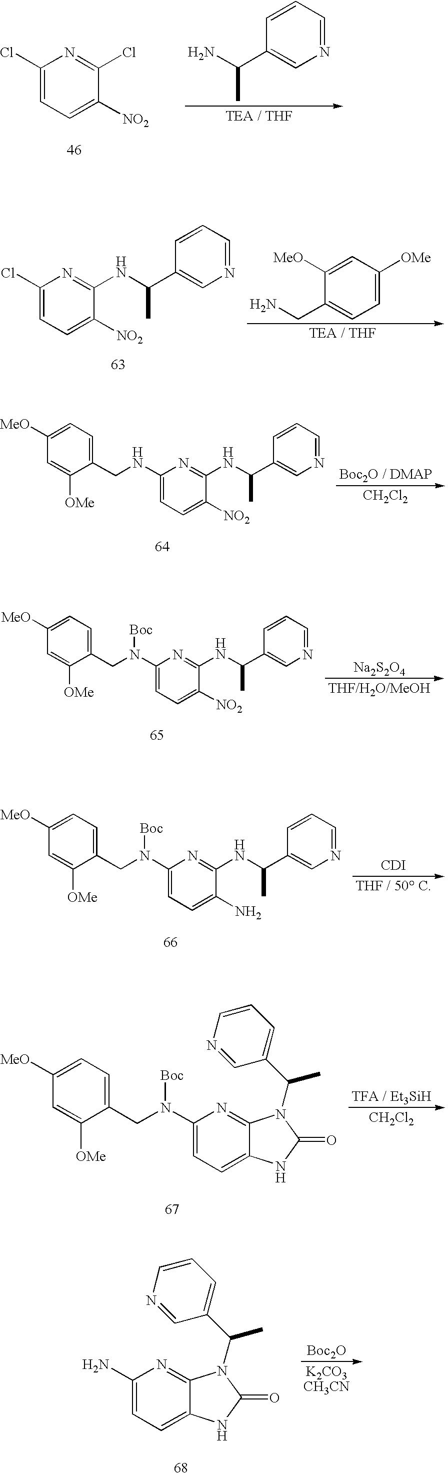

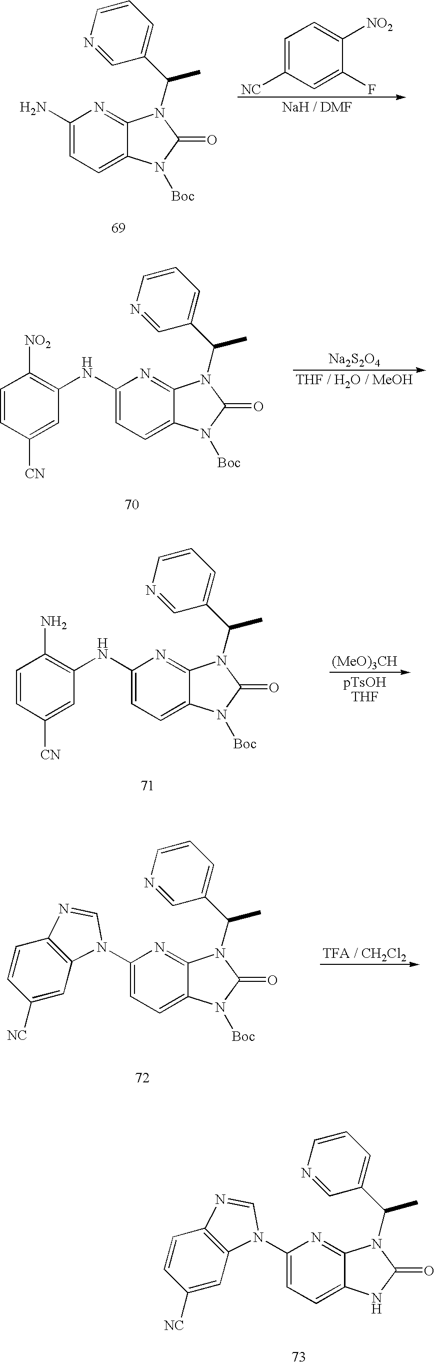

(R)—N6-(2,4-Dimethoxybenzyl)-3-nitro-N2-(1-(pyridin-3-yl)ethyl)pyridine-2,6-diamine (64). A solution of 2,6-dichloro-5-nitropyridine (46) (0.5 g) in THF (20 mL) was cooled to 0° C. and treated with 1.6 mL triethylamine followed by (R)-1-pyridin-3-yl-ethylamine (300 μL). The mixture was stirred for 1.5 h, then warmed to RT and stirred another 20 h. 2,4-Dimethoxybenzylamine (0.8 mL) was added and the mixture was heated at 50° C. for four hours. The mixture was diluted with ethyl acetate and washed twice with saturated sodium chloride solution. The organic layer was separated, dried over sodium sulfate, filtered, and concentrated. Column chromatography (50→100% ethyl acetate in hexanes) provided 761 mg of (R)—N6-(2,4-Dimethoxybenzyl)-3-nitro-N2-(1-(pyridin-3-yl)ethyl)pyridine-2,6-diamine (64).

(R)-tert-Butyl 2,4-dimethoxybenzyl(5-nitro-6-(1-(pyridin-3-yl)ethylamino)pyridin-2-yl)carbamate (65). A solution of (R)—N6-(2,4-dimethoxybenzyl)-3-nitro-N2-(1-(pyridin-3-yl)ethyl)pyridine-2,6-diamine (64) (367 mg) in methylene chloride (20 mL) was treated with di-tert-butyl dicarbonate (1.0 g) and 4-dimethylaminopyridine (22 mg). The mixture was stirred for 16 h and concentrated in vacuo. Column chromatography (50→100% ethyl acetate in hexanes) provided 500 mg of (R)-tert-Butyl 2,4-dimethoxybenzyl(5-nitro-6-(1-(pyridin-3-yl)ethylamino)pyridin-2-yl)carbamate (65).

(R)-tert-Butyl 2,4-dimethoxybenzyl(2-oxo-3-(1-(pyridin-3-yl)ethyl)-2,3-dihydro-1H-imidazo[4,5-b]pyridin-5-yl)carbamate (67). A solution of (R)-tert-butyl 2,4-dimethoxybenzyl(5-nitro-6-(1-(pyridin-3-yl)ethylamino)pyridin-2-yl)carbamate (500 mg) in THF (25 mL) was treated with an aqueous solution comprised of 2 g of Na₂S₂O₄ and 1 g NaHCO₃ in 20 mL of water followed by 1 mL of methanol. The mixture was stirred for 30 minutes, then diluted with ethyl acetate and washed with saturated sodium chloride solution. The organic layer was separated, dried over sodium sulfate, filtered, and concentrated to provide the intermediate (R)-tert-butyl 2,4-dimethoxybenzyl(5-amino-6-(1-(pyridin-3-yl)ethylamino)pyridin-2-yl)carbamate (66). The intermediate was dissolved in THF (50 mL) and treated with 1,1′-carbonyldiimidazole (0.5 g) at 50° C. for 20 h. The mixture was concentrated and purified by column chromatography (2→5% MeOH in methylene chloride) to provide 413 mg of (R)-tert-Butyl 2,4-dimethoxybenzyl(2-oxo-3-(1-(pyridin-3-yl)ethyl)-2,3-dihydro-1H-imidazo[4,5-b]pyridin-5-yl)carbamate (67).

(R)-tert-Butyl 5-amino-2-oxo-3-(1-(pyridin-3-yl)ethyl)-2,3-dihydroimidazo[4,5-b]pyridine-1-carboxylate (69). A solution of (R)-tert-butyl 2,4-dimethoxybenzyl(2-oxo-3-(1-(pyridin-3-yl)ethyl)-2,3-dihydro-1H-imidazo[4,5-b]pyridin-5-yl)carbamate in methylene chloride (15 mL) was treated with TFA (15 mL) and triethylsilane (1.0 mL) for one hour. The mixture was concentrated to provide the intermediate (R)-5-amino-3-(1-(pyridin-3-yl)ethyl)-1H-imidazo[4,5-b]pyridin-2(3H)-one (68), which was dissolved in acetonitrile (50 mL) and stirred vigorously with di-tert-butyl dicarbonate (1.0 g) and potassium carbonate (3.0 g) for 2 h. Methylene chloride (200 mL) and water (100 mL) was added and the organic layer was separated. The aqueous layer was extracted with another 100 mL of methylene chloride. The combined organic layers were separated, dried over sodium sulfate, filtered, and concentrated. Column chromatography (2→3→4% MeOH in methylene chloride) provided 235 mg (R)-tert-Butyl 5-amino-2-oxo-3-(1-(pyridin-3-yl)ethyl)-2,3-dihydroimidazo[4,5-b]pyridine-1-carboxylate (69).

(R)-tert-Butyl 5-(5-cyano-2-nitrophenylamino)-2-oxo-3-(1-(pyridin-3-yl)ethyl)-2,3-dihydroimidazo[4,5-b]pyridine-1-carboxylate (70). A solution of (R)-tert-butyl 5-amino-2-oxo-3-(1-(pyridin-3-yl)ethyl)-2,3-dihydroimidazo[4,5-b]pyridine-1-carboxylate (94 mg) and 3-fluoro-4-nitrobenzonitrile (225 mg) in DMF (6 mL) was cooled to −25° C. and treated with NaH (60% w/w in mineral oil, 75 mg) and slowly allowed to warm to −15° C. The mixture was stirred for four hours between −20° C. and −15° C., then diluted with EtOAc and quenched with saturated ammonium chloride solution. The organic phase was washed three times with brine, separated, dried over sodium sulfate, filtered, and concentrated. Column chromatography (2% MeOH in methylene chloride) provided 100 mg (R)-tert-Butyl 5-(5-cyano-2-nitrophenylamino)-2-oxo-3-(1-(pyridin-3-yl)ethyl)-2,3-dihydroimidazo[4,5-b]pyridine-1-carboxylate (70).

tert-Butyl 5-(6-cyano-1H-benzo[d]imidazol-1-yl)-2-oxo-3-((R)-1-(pyridin-3-yl)ethyl)-2,3-dihydroimidazo[4,5-b]pyridine-1-carboxylate (72). A solution of (R)-tert-butyl 5-(5-cyano-2-nitrophenylamino)-2-oxo-3-(1-(pyridin-3-yl)ethyl)-2,3-dihydroimidazo[4,5-b]pyridine-1-carboxylate (70) (100 mg) in THF (5 mL) was treated with an aqueous solution comprised of 0.5 g of Na₂S₂O₄ and 0.25 g NaHCO₃ in 5 mL of water. The mixture went quickly from a red color to a slightly yellow color, which indicated reduction of the nitro group. The mixture was diluted with ethyl acetate and washed with saturated sodium chloride solution. The organic layer was separated, dried over sodium sulfate, filtered, and concentrated to provide the intermediate (R)-tert-butyl 5-(2-amino-5-cyanophenylamino)-2-oxo-3-(1-(pyridin-3-yl)ethyl)-2,3-dihydroimidazo[4,5-b]pyridine-1-carboxylate (71). The intermediate was dissolved in THF (5 mL), DMF (1 mL), and trimethylorthoformate (2 mL). The mixture was treated with 10 mg of p-toluenesulfonic acid and stirred for 20 h. The mixture was diluted with ethyl acetate and washed once with saturated sodium bicarbonate and twice with saturated NaCl solution. The organic layer was separated, dried over sodium sulfate, filtered, and concentrated. Column chromatography (2% MeOH in methylene chloride) provided 57 mg tert-Butyl 5-(6-cyano-1H-benzo[d]imidazol-1-yl)-2-oxo-3-((R)-1-(pyridin-3-yl)ethyl)-2,3-dihydroimidazo[4,5-b]pyridine-1-carboxylate (72).

3-(2-oxo-3-((R)-1-(pyridin-3-yl)ethyl)-2,3-dihydro-1H-imidazo[4,5-b]pyridin-5-yl)-3H-benzo[d]imidazole-5-carbonitrile (73). A solution of tert-butyl 5-(6-cyano-1H-benzo [d]imidazol-1-yl)-2-oxo-3-((R)-1-(pyridin-3-yl)ethyl)-2,3-dihydroimidazo[4,5-b]pyridine-1-carboxylate (72) (57 mg) in methylene chloride (1 mL) was treated with TFA (1 mL) for one hour. The mixture was concentrated and the resulting TFA salt was converted to the HCl salt by dissolving it in 5 mL EtOH and adding 0.5 mL of conc. HCl, then concentrating the solution in vacuo. The process was repeated and the resulting residue was dissolved in a minimum amount of methanol and triturated with the addition of ethyl ether. After 3 triturations, 3-(2-oxo-3-((R)-1-(pyridin-3-yl)ethyl)-2,3-dihydro-1H-imidazo[4,5-b]pyridin-5-yl)-3H-benzo[d]imidazole-5-carbonitrile (73) HCl salt (39 mg) was isolated as a tan colored solid: ¹H-NMR (CD₃OD) δ 9.9 (br s, 1H), 9.2 (s, 1H), 9.0 (m, 2H), 8.5 (s, 1H), 8.3 (m, 1H), 8.2 (m, 1H), 8.1 (d, 1H), 7.9 (d, 1H), 7.8 (d, 1H), 6.3 (q, 1H), 2.3 (d, 3H).

### Example 24

### Synthesis of 2-(1H-Benzo[d]imidazol-1-yl)-9-(cis-3-methyl-tetrahydro-2H-pyran-4-yl)-7H-purin-8(9H)-one

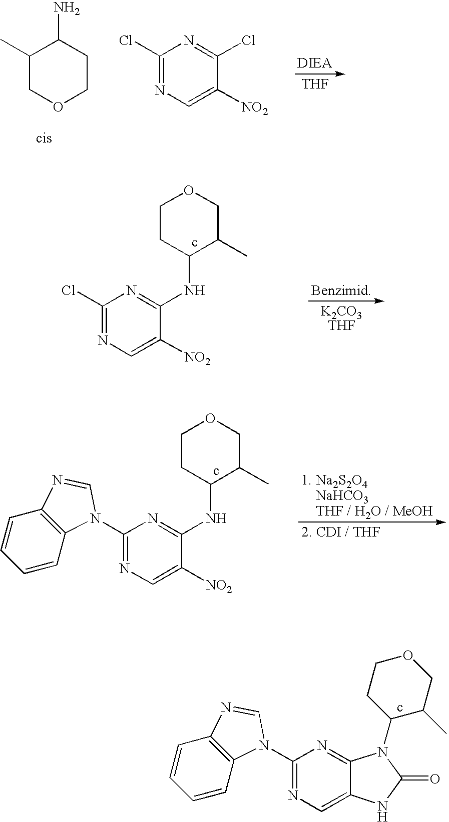

2-Chloro-N-(cis-3-methyl-tetrahydro-2H-pyran-4-yl)-5-nitropyrimidin-4-amine. To a suspension of 0.24 g of the hydrochloride salt of cis-3-methyl-tetrahydro-2H-pyran-4-amine (WO 2004/041161) and DIEA (1.5 mL) in THF (10 mL) at −78° C. was added 2,4-dichloro-5-nitropyrimidine (0.72 g). The mixture was allowed to slowly reach room temperature and stirred for 16 hours. The mixture was diluted with EtOAc and washed 3 times with brine. The organic layer was separated, dried over sodium sulfate, and concentrated in vacuo. Column chromatography (20-40% EtOAc/hexanes) provided 289 mg of the title compound.

2-(1H-Benzo[d]imidazol-1-yl)-N-(cis-3-methyl-tetrahydro-2H-pyran-4-yl)-5-nitropyrimidin-4-amine. To a solution of 2-chloro-N-(cis-3-methyl-tetrahydro-2H-pyran-4-yl)-5-nitropyrimidin-4-amine (115 mg) in acetonitrile (5 mL) was added potassium carbonate (300 mg) and benzimidazole (150 mg). The mixture was stirred at 70° C. for 2.5 hours. After diluting with 70 mL EtOAc, the mixture was washed with brine, dried over sodium sulfate, and concentrated in vacuo. Column chromatography (50→100% EtOAc/hexanes) provided 99 mg of the title compound.

2-(1H-Benzo[d]imidazol-1-yl)-9-(cis-3-methyl-tetrahydro-2H-pyran-4-yl)-7H-purin-8(9H)-one. To a solution of 2-(1H-benzo[d]imidazol-1-yl)-N-(cis-3-methyl-tetrahydro-2H-pyran-4-yl)-5-nitropyrimidin-4-amine (51 mg) in THF (10 mL) was added a solution of sodium hydrosulfite (300 mg) and sodium bicarbonate (150 mg) in water (10 mL). The mixture briefly became blue followed by colorless. Methanol (1 mL) was added to maintain the homogeneity of the solution. The mixture was diluted with 70 mL EtOAc and washed twice with brine. The aqueous washes were extracted with another 50 mL of EtOAc and then the combined organic layers were dried over sodium sulfate and concentrated in vacuo to provide 2-(1H-benzo[d]imidazol-1-yl)-N4-(cis-3-methyl-tetrahydro-2H-pyran-4-yl)pyrimidine-4,5-diamine. The diamine intermediate was dissolved in THF (5 mL) and treated with 1,1′-carbonyldiimidazole (80 mg) at 50° C. for 16 hours. The mixture was diluted with 50 mL EtOAc and washed 3 times with brine. The organic layer was separated, dried over sodium sulfate, and concentrated in vacuo. Column chromatography (2→4% MeOH/DCM) provided 19.3 mg of the title compound. ¹H-NMR (300 MHz, 5% CD₃OD in CDCl₃) δ 8.9 (s, 1H), 8.5 (d, 1H), 8.2 (s, 1H), 7.8 (d, 1H), 7.4 (t, 1H), 7.3 (t, 1H), 4.7 (m, 1H), 4.2 (d(br), 1H), 3.9 (d, 1H), 3.7 (d, 1H), 3.5 (m, 2H), 2.3 (t(br), 1H), 1.8 (d(br), 1H), 1.2 (d, 3H).

### Example 25

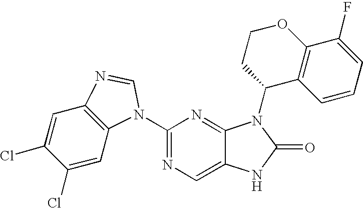

2-(5,6-Dichloro-1H-benzo[d]imidazol-1-yl)-9-((R)-8-fluorochroman-4-yl)-7H-purin-8(9H)-one. A solution of (R)-2-chloro-N-(8-fluorochroman-4-yl)-5-nitropyrimidin-4-amine in acetonitrile was treated with 5,6-dichlorobenzimidazole and potassium carbonate. The mixture was stirred at reflux for 6 hours, cooled to room temperature, diluted with 150 mL of EtOAc, and washed twice with 30 mL portions of water. The organic layer was separated, dried with magnesium sulfate, filtered, and concentrated in vacuo. Purification by column chromatography (2% MeOH/DCM) gave the intermediate nitropyrimidinamine. The title compound was synthesized from the intermediate nitropyrimidinamine via the procedures described in Example 24. ¹H-NMR (300 MHz, CDCl₃) δ 8.7 (s, 1H), 8.3 (s, 1H), 8.2 (s, 1H), 7.8 (t, 1H), 7.0 (t, 1H), 6.6 (m, 2H), 5.9 (t, 1H), 4.6 (m, 1H), 4.4 (m, 1H), 3.0 (m, 1H), 2.3 (m, 1H).

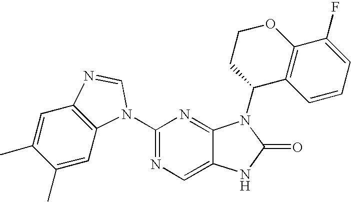

2-(5,6-Dimethyl-1H-benzo [d]imidazol-1-yl)-9-((R)-8-fluorochroman-4-yl)-7H-purin-8(9H)-one. The title compound was synthesized from 5,6-dichlorobenzimidazole via the procedures described in Example 25. ¹H-NMR (300 MHz, CDCl₃) δ 9.6 (s, 1H), 8.8 (s, 1H), 8.3 (s, 1H), 8.0 (s, 1H), 7.6 (s, 1H), 7.0 (t, 1H), 6.7 (m, 1H), 5.9 (t, 1H), 4.7 (m, 1H), 4.5 (m, 1H), 3.2 (m, 2H), 2.4 (d, 6H).

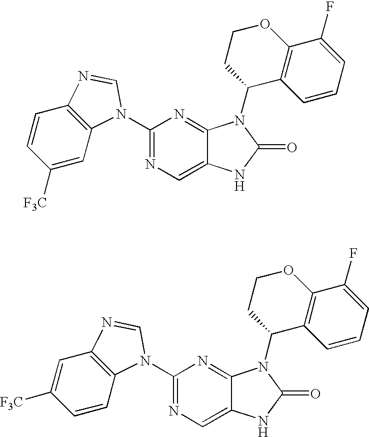

9-((R)-8-Fluorochroman-4-yl)-2-(6-(trifluoromethyl)-1H-benzo[d]imidazol-1-yl)-7H-purin-8(9H)-one and 9-((R)-8-fluorochroman-4-yl)-2-(5-(trifluoromethyl)-1H-benzo[d]imidazol-1-yl)-7H-purin-8(9H)-one. The title compound was synthesized from 5-trifluoromethylbenzimidazole (US 2004/0087601) via the procedures described in Example 25. Purification by column chromatography (2% MeOH/DCM) eluted the 6-trifluoromethyl isomer first (¹H-NMR (300 MHz, CDCl₃) δ 8.8 (d, 2H), 8.4 (s, 1H), 7.9 (d, 1H), 7.6 (d, 2H), 7.0 (t, 1H), 6.7 (m, 2H), 5.9 (t, 1H), 4.7 (m, 1H), 4.4 (m, 1H), 3.0 (m, 1H), 2.4 (m, 1H).) followed by the 5-trifluoromethyl isomer (¹H-NMR (300 MHz, CDCl₃) δ 9.0 (s, 1H), 8.4 (s, 1H), 8.1 (s, 1H), 8.0 (d, 1H), 7.6 (d, 2H), 7.0 (m, 1H), 6.8 (m, 2H), 5.9 (t, 1H), 4.7 (m, 1H), 4.4 (m, 1H), 2.9 (m, 1H), 2.4 (m, 1H).

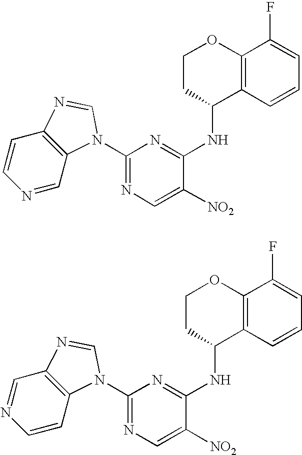

N—((R)-8-Fluorochroman-4-yl)-2-(3H-imidazo[4,5-c]pyridin-3-yl)-5-nitropyrimidin-4-amine and N—((R)-8-fluorochroman-4-yl)-2-(1H-imidazo[4,5-c]pyridin-1-yl)-5-nitropyrimidin-4-amine. The title compound was synthesized from 5-azabenzimidazole via the procedure described in Example 25. Purification by column chromatography (1% MeOH/DCM) provided N—((R)-8-fluorochroman-4-yl)-2-(3H-imidazo[4,5-c]pyridin-3-yl)-5-nitropyrimidin-4-amine as the first eluting isomer: (¹H-NMR (300 MHz, CDCl₃) δ 9.8 (s, 1H), 9.4 (s, 1H), 9.2 (s, 1H), 8.9 (d, 1H), 8.6 (d, 1H), 7.8 (d, 1H), 7.1 (m, 2H), 6.9 (m, 1H), 5.8 (q, 1H), 4.6 (m, 1H), 4.4 (m, 1H), 2.6 (m, 1H), 2.4 (m, 1H).). N—((R)-8-Fluorochroman-4-yl)-2-(1H-imidazo[4,5-c]pyridin-1-yl)-5-nitropyrimidin-4-amine eluted second: (¹H-NMR (300 MHz, CDCl₃) δ 9.4 (s, 1H), 9.2 (s, 1H), 9.1 (s, 1H), 8.9 (d, 1H), 8.6 (d, 1H), 8.4 (d, 1H), 7.1 (m, 2H), 6.9 (m, 1H), 5.7 (q, 1H), 4.5 (m, 1H), 4.4 (m, 1H), 2.6 (m, 1H), 2.4 (m, 1H).).

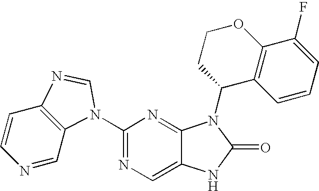

9-((R)-8-fluorochroman-4-yl)-2-(3H-imidazo[4,5-c]pyridin-3-yl)-7H-purin-8(9H)-one. The title compound was synthesized from N—((R)-8-fluorochroman-4-yl)-2-(3H-imidazo[4,5-c]pyridin-3-yl)-5-nitropyrimidin-4-amine via the procedures described in Example 24. ¹H-NMR (300 MHz, CDCl₃) δ 9.8 (s, 1H), 9.4 (s, 1H), 8.6 (d, 1H), 8.3 (m, 2H), 7.0 (t, 1H), 6.7 (m, 2H), 5.9 (t, 1H), 4.6 (m, 1H), 4.4 (m, 1H), 2.8 (m, 1H), 2.4 (m, 1H).

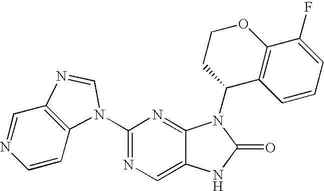

9-((R)-8-fluorochroman-4-yl)-2-(1H-imidazo[4,5-c]pyridin-1-yl)-7H-purin-8(9H)-one. The title compound was synthesized from N—((R)-8-fluorochroman-4-yl)-2-(1H-imidazo[4,5-c]pyridin-1-yl)-5-nitropyrimidin-4-amine via the procedures described in Example 24. ¹H-NMR (300 MHz, CDCl₃) δ 9.3 (d, 2H), 8.4 (d, 1H), 8.3 (d, 2H), 7.0 (t, 1H), 6.7 (m, 2H), 5.9 (t, 1H), 4.6 (m, 1H), 4.4 (m, 1H), 2.8 (m, 1H), 2.4 (m, 1H).

### Example 26

### Synthesis of 3-(9-((R)-6,8-difluorochroman-4-yl)-8-oxo-8,9-dihydro-7H-purin-2-yl)-3H-benzo[d]imidazole-5-carbonitrile

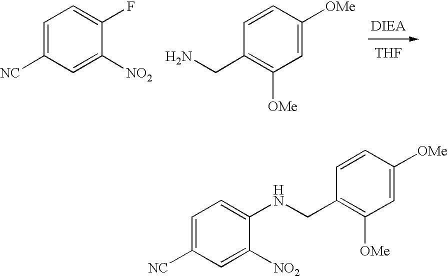

4-(2,4-dimethoxybenzylamino)-3-nitrobenzonitrile. A solution of 4-fluoro-3-nitrobenzonitrile (5.0 g) in THF (100 mL) was treated with DIEA (6.3 mL) and 2,4-dimethoxybenzylamine (5.0 mL), and then stirred for 24 h. The solvent was evaporated and the crude mixture was dissolved in EtOAc (100 mL). The solution was washed once with 1 M HCl and twice with saturated aqueous NaCl (100 mL each). The organic layer was separated, dried over Na₂SO₄, filtered, and concentrated in vacuo. Column chromatography (20% EtOAc/DCM) provided 9.25 g of the title compound.

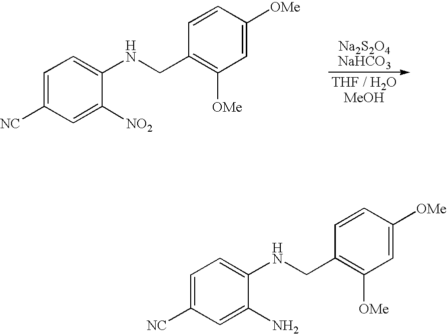

4-(2,4-dimethoxybenzylamino)-3-aminobenzonitrile. A solution of 4-(2,4-dimethoxybenzylamino)-3-nitrobenzonitrile (4.54 g) in THF (400 mL) was treated with a solution of sodium hydrosulfite (20 g) and sodium bicarbonate (10 g) in distilled water (350 mL). Enough methanol was immediately added (50 mL) to maintain a homogeneous solution. After 15 minutes, EtOAc (500 mL) and saturated aqueous NaCl (500 mL) were added and the organic layer was separated. The aqueous layer was extracted again with 400 mL EtOAc. The combined organic layers were washed with saturated aqueous NaCl (500 mL) and separated. The organic phase was dried over Na₂SO₄, filtered, and concentrated in vacuo to provide 4.33 g of the title compound.

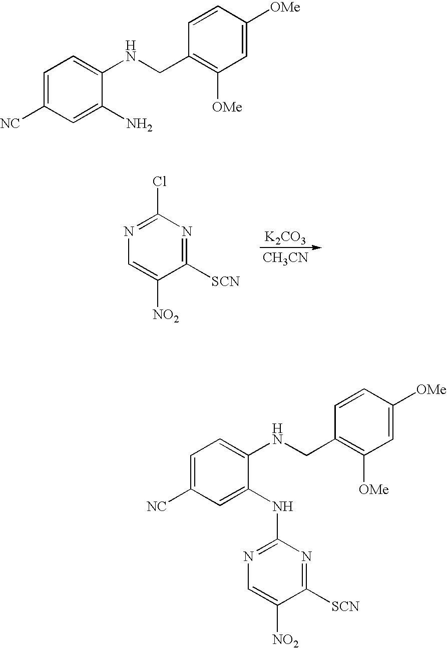

4-(2,4-dimethoxybenzylamino)-3-(5-nitro-4-thiocyanatopyrimidin-2-ylamino)benzonitrile. A solution of 4-(2,4-dimethoxybenzylamino)-3-aminobenzonitrile (3.9 g) in acetonitrile (100 mL) was cooled to 0° C. and treated with potassium carbonate (6.3 g) followed by a solution containing 3 g of 2-chloro-5-nitro-4-thiocyanatopyrimidine (WO 2003/032994) in acetonitrile (50 mL). The mixture was stirred for 30 minutes at 0° C. and 30 minutes at room temperature resulting in the formation of a precipitate. The mixture was quenched at 0° C. by the addition of 4% acetic acid (150 mL) and filtered. The precipitate was swirled in 100 mL acetonitrile and filtered again. The precipitate was washed with acetonitrile, which resulted in the slow dissolution of product into the filtrate. After air-drying, 1.5 g of the title compound remained as the precipitate cake. The filtrate was extracted with EtOAc, dried over Na₂SO₄, filtered, and concentrated in vacuo. Column chromatography (0→20% EtOAc/DCM) and recrystallization from acetonitrile provided 0.415 g of additional title compound.

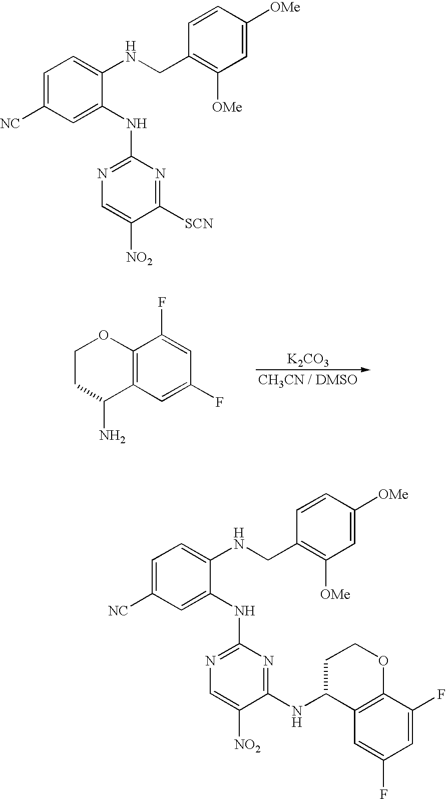

(R)-4-(2,4-dimethoxybenzylamino)-3-(4-(6,8-difluorochroman-4-ylamino)-5-nitropyrimidin-2-ylamino)benzonitrile. A partial suspension of 4-(2,4-dimethoxybenzylamino)-3-(5-nitro-4-thiocyanatopyrimidin-2-ylamino)benzonitrile (415 mg) in 40 mL of acetonitrile was treated with a solution of (R)-6,8-difluorochroman-4-amine HCl salt (320 mg) in DMSO (10 mL) followed by potassium carbonate (1.0 g). The mixture was stirred for 24 hours, and then diluted with EtOAc (200 mL). The mixture was washed once with saturated aqueous ammonium chloride (200 mL) and 3 times with saturated aqueous NaCl (200 mL each). The organic layer was separated, dried over Na₂SO₄, filtered, and concentrated in vacuo. Column chromatography (20→40% EtOAc/hexanes) provided 358 mg of the title compound.

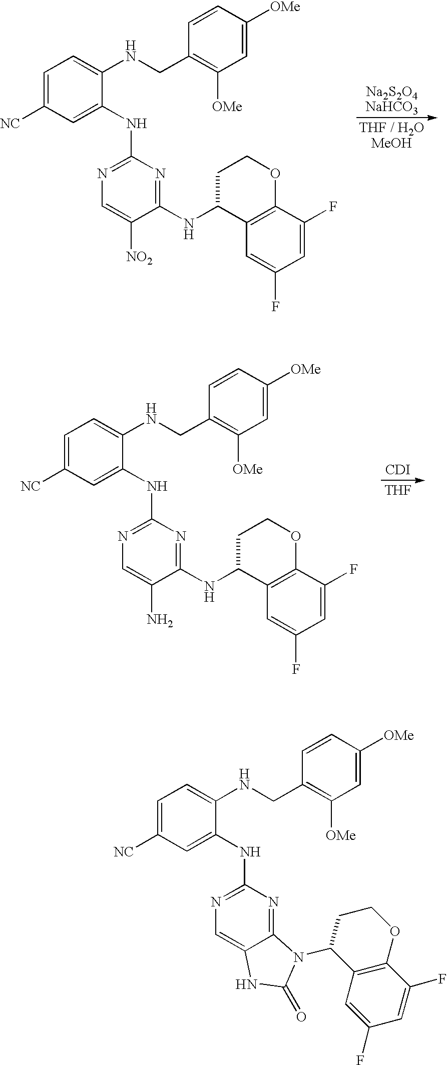

(R)-4-(2,4-dimethoxybenzylamino)-3-(9-(6,8-difluorochroman-4-yl)-8-oxo-8,9-dihydro-7H-purin-2-ylamino)benzonitrile. A solution of (R)-4-(2,4-dimethoxybenzylamino)-3-(4-(6,8-difluorochroman-4-ylamino)-5-nitropyrimidin-2-ylamino)benzonitrile (358 mg) in THF (25 mL) was treated with a solution of sodium hydrosulfite (1.5 g) and sodium bicarbonate (1.5 g) in 20 mL of distilled water. Methanol (5 mL) was added to maintain a homogeneous solution. After 15 minutes, the mixture was diluted with EtOAc (100 mL) and washed with saturated aqueous NaCl (2×100 mL). The organic layer was separated, dried over Na₂SO₄, filtered, and concentrated in vacuo to provide the intermediate (R)-4-(2,4-dimethoxybenzylamino)-3-(5-amino-4-(6,8-difluorochroman-4-ylamino)pyrimidin-2-ylamino)benzonitrile. The intermediate was dissolved in THF (5 mL) and treated with carbonyldiimidazole (0.55 g) for 16 hours. The mixture was diluted with EtOAc (100 mL) and washed twice with saturated aqueous NaCl (2×100 mL). The organic layer was separated, dried over Na₂SO₄, filtered, and concentrated in vacuo. Column chromatography (2→3% MeOH/DCM) provided 230 mg of the title compound.

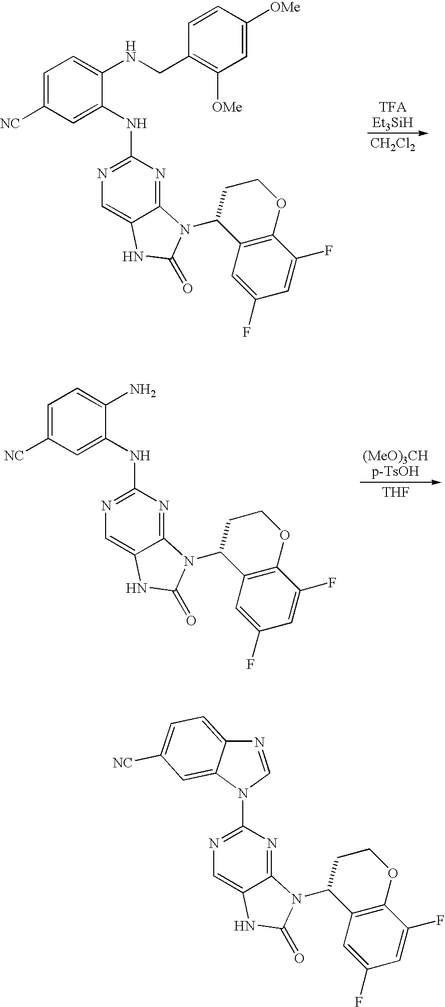

3-(9-((R)-6,8-difluorochroman-4-yl)-8-oxo-8,9-dihydro-7H-purin-2-yl)-3H-benzo[d]imidazole-5-carbonitrile. A solution of (R)-4-(2,4-dimethoxybenzylamino)-3-(9-(6,8-difluorochroman-4-yl)-8-oxo-8,9-dihydro-7H-purin-2-ylamino)benzonitrile (230 mg) in DCM (5 mL) was treated with TFA (5 mL) and triethylsilane (1 mL) for 16 h. The mixture was concentrated in vacuo to provide the intermediate (R)-4-amino-3-(9-(6,8-difluorochroman-4-yl)-8-oxo-8,9-dihydro-7H-purin-2-ylamino)benzonitrile. The intermediate was dissolved in 5 mL THF and treated with 3 mL trimethylorthoformate followed by p-toluenesulfonic acid (3 mg). After 1 hour, the mixture was diluted with EtOAc (100 mL) and washed once with saturated aqueous sodium bicarbonate (100 mL). The organic layer was separated, dried over Na₂SO₄, filtered, and concentrated in vacuo. Column chromatography (50→100% EtOAc/hexanes) provided 78 mg of the title compound. ¹H-NMR (300 MHz, 5% CD₃OD in CDCl₃) δ 8.8 (s, 1H), 8.7 (s, 1H), 8.2 (s, 1H), 7.8 (d, 1H), 7.6 (dd, 1H), 6.8 (td, 1H), 6.4 (dd, 1H), 5.8 (dd, 1H), 4.6 (m, 1H), 4.4 (td, 1H), 2.9 (m, 1H), 2.3 (m, 1H).

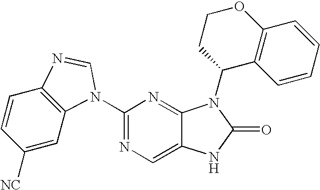

3-(9-((R)-chroman-4-yl)-8-oxo-8,9-dihydro-7H-purin-2-yl)-3H-benzo[d]imidazole-5-carbonitrile. The title compound was synthesized from (R)-chroman-4-amine via the procedures described in Example 26. ¹H-NMR (300 MHz, 5% CD₃OD in CDCl₃) δ 8.8 (s, 1H), 8.5 (s, 1H), 8.2 (s, 1H), 7.8 (d, 1H), 7.5 (dd, 1H), 7.1 (m, 2H), 6.8 (d, 1H), 6.7 (td, 1H), 5.8 (dd, 1H), 4.5 (m, 1H), 4.3 (td, 1H), 2.8 (m, 1H), 2.3 (m, 1H).

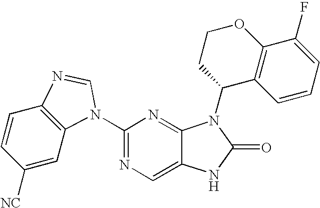

3-[9-(8-Fluoro-chroman-4-yl)-8-oxo-8,9-dihydro-7H-purin-2-yl]-3H-benzoimidazole-5-carbonitrile. The title compound was synthesized from (R)-8-fluorochroman-4-amine via the procedures described in Example 26. ¹H-NMR (300 MHz, CDCl₃) δ 8.8 (s, 1H), 8.6 (s, 1H), 8.2 (s, 1H), 7.8 (d, 1H), 7.6 (d, 1H), 7.0 (t, 1H), 6.6 (m, 2H), 5.8 (t, 1H), 4.6 (m, 1H), 4.4 (m, 1H), 2.8 (m, 1H), 2.4 (m, 1H).

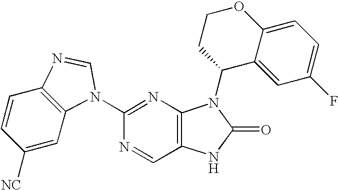

3-(9-((R)-6-fluorochroman-4-yl)-8-oxo-8,9-dihydro-7H-purin-2-yl)-3H-benzo [d]imidazole-5-carbonitrile. The title compound was synthesized from (R)-6-fluorochroman-4-amine via the procedures described in Example 26. ¹H NMR (300 MHz, CDCl₃+5% CD₃OD): δ 8.86 (s, 1H), 8.41 (s, 1H), 8.18 (s, 1H), 7.72 (d, 1H), 7.51 (d, 1H), 7.0-7.1 (m, 1H), 6.8-6.9 (m, 1H), 6.49 (dd, 1H), 5.76 (br t, 1H), 4.4-4.5 (m, 1H), 4.24 (br t, 1H), 2.7-2.9 (m, 1H), 2.2-2.3 (m, 1H). Conditions for introduction of the chromanyl amine were improved as described below:

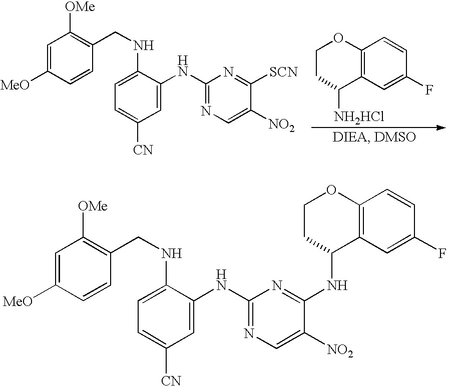

(R)-4-(2,4-dimethoxybenzylamino)-3-(4-(6-fluorochroman-4-ylamino)-5-nitropyrimidin-2-ylamino)benzonitrile. A solution of 4-(2,4-dimethoxybenzylamino)-3-(5-nitro-4-thiocyanatopyrimidin-2-ylamino)benzonitrile (139 mg) in anhydrous DMSO (3 mL) was added to solution of (R)-6-fluorochroman-4-amine hydrochloride (79 mg) in anhydrous DMSO (3 mL) and DIEA (0.21 mL), the resulting dark red solution was stirred at RT under an atmosphere of Ar over which time the solution lighten to yellow. Upon completion of the reaction, the mixture was cooled to 0° C. with an ice bath, and water (25 mL) was added (exotherm). The resulting yellow solid was collected via filtration, washed with additional water, air dried, then dissolved in CH₂Cl₂, the organic solution was dried (MgSO₄), filtered and evaporated to yield the titled compound (quant.), NMR CDCl₃ ¹H δ 9.0 (s, 1H), 8.6(d, 1H), 7.7 (br s, 1H), 7.4 (dd, 1H), 7.1 (d, 1H), 7.0-6.8 (m, 4H), 6.5-6.4 (m, 2H), 5.2 (br s, 1H), 4.3 (s, 2H), 4.2 (br s, 2H), 3.8 (s, 6H), 2.2 (br s, 1H), 1.8 (br s, 1H); ¹⁹F δ −123 ppm; MH⁺=572.

This material was taken on using the same procedures outlined in Example 26 to give 3-(9-((R)-6-fluorochroman-4-yl)-8-oxo-8,9-dihydro-7H-purin-2-yl)-3H-benzo[d]imidazole-5-carbonitrile.

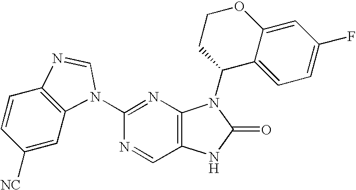

3-(9-((R)-7-fluorochroman-4-yl)-8-oxo-8,9-dihydro-7H-purin-2-yl)-3H-benzo[d]imidazole-5-carbonitrile. The title compound was synthesized from (R)-7-fluorochroman-4-amine via the procedures described in Example 26. ¹H NMR (300 MHz, CDCl₃+5% CD₃OD): δ 8.86 (s, 1H), 8.58 (s, 1H), 8.19 (s, 1H), 7.79 (d, 1H), 7.56 (d, 1H), 6.7-6.9 (m, 2H), 6.4-6.5 (m, 1H), 5.78 (br t, 1H), 4.5-4.6 (m, 1H), 4.32 (br t, 1H), 2.7-2.9 (m, 1H), 2.2-2.4 (m, 1H).

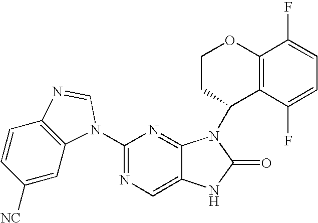

3-[9-(5,8-Difluoro-chroman-4-yl)-8-oxo-8,9-dihydro-7H-purin-2-yl]-3H-benzoimidazole-5-carbonitrile. The title compound was synthesized from (R)-5,8-difluorochroman-4-amine via the procedures described in Example 26. ¹H-NMR (300 MHz, CDCl₃) δ 8.8 (s, 1H), 8.7 (s, 1H), 8.2 (s, 1H), 7.8 (d, 1H), 7.6 (d, 1H), 7.0 (m, 1H), 6.4 (m, 1H), 5.9 (t, 1H), 4.6 (m, 1H), 4.4(m, 1H), 2.5(m, 2H).

### Example 27

### Synthesis of 2-(6-fluoro-1H-benzo[d]imidazol-1-yl)-9-((R)-8-fluoro-chroman-4-yl)-7H-purin-8(9H)-one

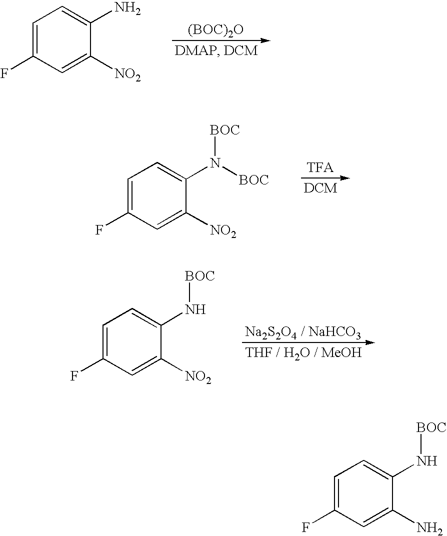

4-Fluoro-2-nitro-phenyl di-tert-butyl imidodicarbonate. A catalytic amount of DMAP was added to a mixture of 4-fluoro-2-nitrobenzenamine (0.78 g) and di-tert-butyl dicarbonate (2.18 g) in DCM (20 mL) and stirred at room temperature for 15 hr. The mixture was diluted with H₂O and twice extracted with DCM, the combined organics were dried, filtered and evaporated to yield the bis-BOC material (quant). ¹H-NMR (300 MHz, CDCl₃) δ 7.8 (dd, 1H), 7.3 (m, 2H), 1.4 (s, 18H).

tert-Butyl 4-fluoro-2-nitrophenylcarbamate. (procedure: Connell, R. D.; Rein, T.; Akermark, B.; Helquist, P. J. J. Org. Chem. 1988, 53, 3845) To a stirred solution of the Bis-BOC material in DCM (20 mL) was added TFA (0.58 mL). After 3 hr the reaction was quenched with aq. NaHCO₃ (5 mL), brine was added, the mixture separated and extracted with additional DCM. The combined organics were evaporated, purified via column chromatography (eluted with 7.5% EtOAc/Hex) to give the titled product (1.12 g). ¹H-NMR (300 MHz, CDCl₃) δ 9.5 (br 1H), 8.5 (dd, 1H), 7.9 (dd, 1H), 7.3 (m, 1H), 1.5 (s, 9H).

tert-Butyl 2-amino-4-fluorophenylcarbamate. To a solution of tert-butyl 4-fluoro-2-nitrophenylcarbamate (0.34 g) in THF (30 mL) was added a premixed solution of sodium hydrosulfite (2 g) and sodium bicarbonate (1 g) in water (50 mL). MeOH (10 mL) was also added to aid solution of the mixture, which was stirred at room temperature for 30 min, when sodium chloride was added to saturate the solution. The resultant mixture was extracted with EtOAc (2×). The combined organics were dried, filtered and evaporated to yield the titled compound (quant) that was used as such for the next step. ¹H-NMR (300 MHz, CDCl₃) δ 7.5 (dd, 1H), 6.6 (dd, 1H), 6.5 (m, 1H), 6.4 (br 1H), 4.7 (br 2H), 1.5 (s, 9H); MH⁺=227 (minor) 127 (—BOC), 171 (-tBu).

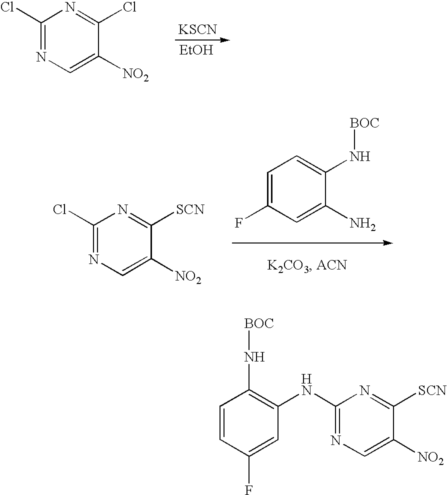

2-Chloro-5-nitro-4-thiocyanatopyrimidine. (compound known, e.g. WO 2003/032994) Potassium thiocyanate (0.97 g, 10 mM) was added to a solution of 2,4-dichloro-5-nitropyrimidine (1.94 g 10, mM) in EtOH (40 mL) cooled to 0° C. via an ice bath. The solution was stirred at 0° C. for 30 min, then the bath was removed and the resulting suspension allowed to come to RT over 60 min, when water (100 mL) was added. The precipitate was collected via filtration, washed with ice cold water, dissolved with DCM, dried (MgSO₄), filtered and evaporated to yield the titled compound (1.7 g). ¹H-NMR (300 MHz, CDCl₃) δ 9.4 (s, 1H).

tert-Butyl 4-fluoro-2-(5-nitro-4-thiocyanatopyrimidin-2-ylamino)phenylcarbamate. Potassium carbonate (207 mg) was added to a stirred solution of 2-chloro-5-nitro-4-thiocyanatopyrimidine (108 mg) and tert-butyl 4-fluoro-2-nitrophenylcarbamate (113 mg) in ACN (5 mL) and stirred for 15 hr. The solution was diluted with brine and extracted with EtOAc (2×). The combined organics were evaporated and purified via column chromatography, elution with 30% EtOAc/Hex gave the titled compound (144 mg, 71% yield). ¹H-NMR (300 MHz, DMSO-d₆) δ 10.5 (br s, 1H), 9.3 (br s, 1H), 8.9 (br s, 1H), 7.7-7.4 (m, 2H), 7.1 (br s, 1H), 1.5 (s, 9H), 1.5 (s, 9H); MH⁺=407, 307 (—BOC), 351 (-tBu).

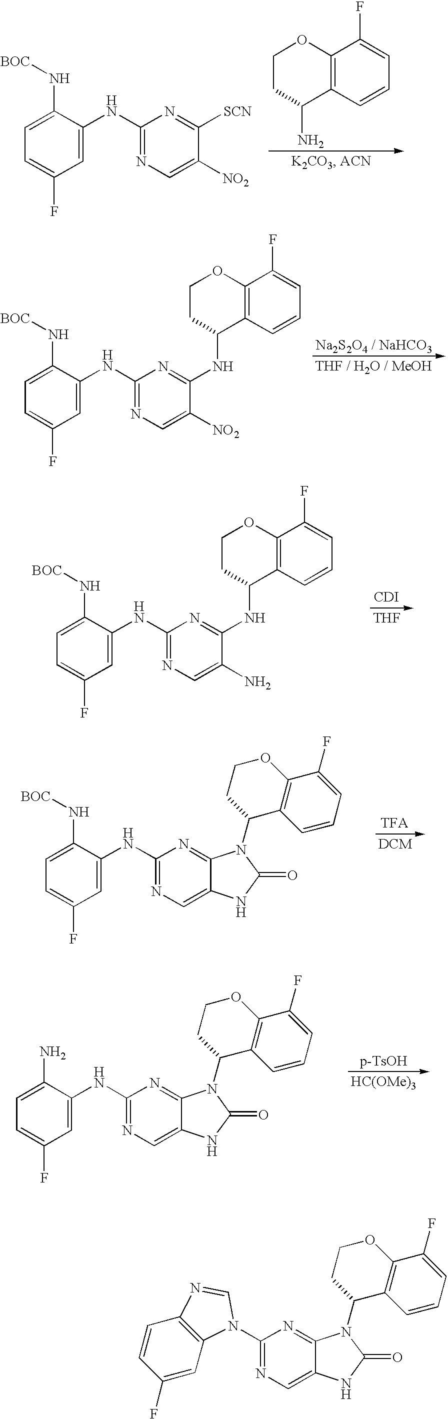

(R)-tert-Butyl 4-fluoro-2-(4-(8-fluorochroman-4-ylamino)-5-nitropyrimidin-2-ylamino)phenylcarbamate. A solution of the (R)-8-fluorochroman-4-amine hydrochloride (104 mg) in DMSO (2 mL) and potassium carbonate (141 mg) were added to a stirred solution of tert-butyl 4-fluoro-2-(5-nitro-4-thiocyanatopyrimidin-2-ylamino)phenylcarbamate (140 mg) in ACN (10 mL). The mixture was stirred for 15 hr at room temperature then partitioned between brine and EtOAc and separated. The aq. layer was washed with additional EtOAc, the combined organics were evaporated and purified via column chromatography, elution with 20-30% EtOAc/H gave the titled product in 83% yield. ¹H-NMR (300 MHz, CDCl₃) δ 9.1 (s, 1H), 8.7(m, 1H), 8.2 (br s, 1H), 7.7 (m, 1H), 7.3 (m, 1H), 7.3-6.8 (m, 4H), 6.5 (s, 1H), 5.5 (br s, 1H), 4.4 (m 2H), 2.4 (m, 1H), 2.2 (m, 1H), 1.5 (s, 9H); MH⁺=515, 459 (-tBu).

(R)-tert-Butyl 4-fluoro-2-(9-(8-fluorochroman-4-yl)-8-oxo-8,9-dihydro-7H-purin-2-ylamino)phenylcarbamate. To a solution of (R)-tert-butyl 4-fluoro-2-(4-(8-fluorochroman-4-ylamino)-5-nitropyrimidin-2-ylamino)phenylcarbamate (141 mg) in THF (20 mL) was added a premixed solution of sodium hydrosulfite (0.6 g) and sodium bicarbonate (0.3 g) in water (50 mL). MeOH (5 mL) was also added to aid solution of the mixture, which was stirred at room temperature for 30 min, when sodium chloride was added to saturate the solution. The resultant mixture was extracted with EtOAc (2×), the combined organics were dried, filtered and evaporated to yield (R)-tert-butyl 2-(5-amino-4-(8-fluorochroman-4-ylamino)pyrimidin-2-ylamino)-4-fluorophenylcarbamate that was used as such for the next step, MH⁺=485.

To a stirred solution of the above material in THF (5 mL) was added CDI (131 mg). After 15 hr brine and EtOAc were added and the mixture was separated. The aq. layer was washed with additional EtOAc and the combined organics were evaporated and purified by column chromatography (eluted 3% MeOH/DCM) to yield titled product (86 mg, 62% yield for two steps). ¹H-NMR (300 MHz, 5% CD₃OD in CDCl₃) δ 7.9 (s, 1H), 7.4 (dd, 1H), 7.3 (m, 1H), 6.9 (dd, 1H), 6.7-6.5 (m, 3H), 5.7 (dd,1H), 4.6 (m 1H), 4.3(td, 1H), 2.9 (m, 1H), 2.2 (m, 1H), 1.5 (s, 9H); MH⁺=511, 411 (—BOC), 455 (-tBu).

2-(6-Fluoro-1H-benzo[d]imidazol-1-yl)-9-((R)-8-fluorochroman-4-yl)-7H-purin-8(9H)-one. A freshly prepared solution of 30% TFA/DCM (5 mL) was added to (R)-tert-butyl 4-fluoro-2-(9-(8-fluorochroman-4-yl)-8-oxo-8,9-dihydro-7H-purin-2-ylamino)phenylcarbamate and the solution was stirred at room temperature for 60 min then the solvents were removed in vacuo to yield (R)-2-(2-amino-5-fluorophenylamino)-9-(8-fluorochroman-4-yl)-7H-purin-8(9H)-one that was used as such MH⁺=411.

To the above di-amine was added MeOH (2 mL), trimethylorthoformate (2 mL) and p-TsOH (cat). The mixture was stirred at RT for 60 min then the solvents were reduced and the resultant material partitioned between DCM and brine and separated. The crude product was purified via column chromatography (eluted with 4% MeOH/DCM) to yield the titled compound (46 mg). ¹H-NMR (300 MHz, 5% CD₃OD in CDCl₃) δ 8.7 (s, 1H), 8.1 (s, 1H), 7.5 (m, 2H), 6.9 (m, 2H), 6.6 (m, 2H), 5.8 (dd, 1H), 4.6 (m 1H), 4.3 (td, 1H), 2.8 (m, 1H), 2.3 (m, 1H); MH⁺=421.

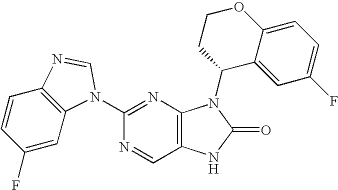

2-(6-Fluoro-1H-benzo [d]imidazol-1-yl)-9-((R)-6-fluorochroman-4-yl)-7H-purin-8(9H)-one. The title compound was synthesized from (R)-6-fluorochroman-4-amine via the procedures described in Example 27. ¹H NMR (300 MHz, CDCl₃+5% CD₃OD): δ 8.72 (s, 1H), 8.16 (s, 1H), 7.5-7.7 (m, 2H), 6.9-7.0 (m, 2H), 6.8-6.9 (m, 1H), 6.54 (dd, 1H), 5.78 (br t, 1H), 4.4-4.5 (m, 1H), 4.26 (m, 1H), 2.7-2.8 (m, 1H), 2.2-2.3 (m, 1H).

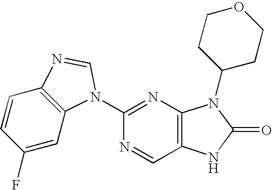

2-(6-Fluoro-1H-benzo [d]imidazol-1-yl)-9-(tetrahydro-2H-pyran-4-yl)-7H-purin-8(9H)-one. The title compound was synthesized from 4-aminotetrahydropyran via the procedures described in Example 27. ¹H NMR (d₆-DMSO) δ 11.65 (s, 1H), 9.13 (s, 1H), 8.34 (s, 1H), 8.28 (m, 1H), 7.82 (m, 1H), 7.25 (td, J=9.0, 2.4 Hz, 1H), 4.56 (m, 1H), 4.03 (dd, J=11.1, 3.9 Hz, 2H), 3.50 (t, J=11.1 Hz, 2H), 2.59 (m, 2H), 1.78 (m, 2H).

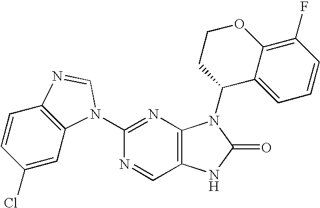

2-(6-Chloro-1H-benzo[d]imidazol-1-yl)-9-((R)-8-fluorochroman-4-yl)-7H-purin-8(9H)-one. The title compound was synthesized from (R)-8-fluorochroman-4-amine and 4-chloro-2-nitrobenzenamine via the procedures described in Example 27. ¹H NMR (300 MHz, CDCl₃+5% CD₃OD): δ 8.7 (s, 1H), 8.2 (s, 1H), 8.1 (s, 1H), 7.6 (d, 1H), 7.2 (dd, 1H), 6.9 (td, 1H), 6.7-6.5 (m, 2H). 5.8 (dd, 1H), 4.6 (m 1H), 4.4 (td, 1H), 2.9 (m, 1H), 2.3 (m, 1H).

### Example 28

### Synthesis of 2-(5-Fluoro-1H-benzo[d]imidazol-1-yl)-9-((R)-8-fluorochroman-4-yl)-7H-purin-8(9H)-one

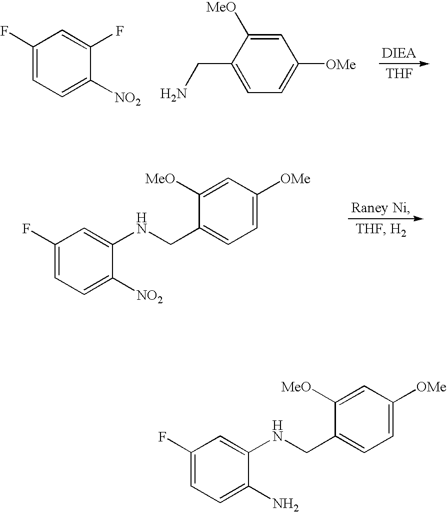

N-(2,4-Dimethoxybenzyl)-5-fluoro-2-nitrobenzenamine. A solution of 2,4-difluoro-1-nitrobenzene (1.1 mL), 2,4-dimethoxy benzylamine (1.5 mL) and DIEA (5.2 mL), in THF (40 mL) was heated at 60° C. for 60 min, allowed to cool to RT, partitioned between EtOAc and H₂O, separated, dried (MgSO₄), filtered and evaporated to yield the titled product as a yellow solid (3.14 g). ¹H-NMR (300 MHz, CDCl₃) δ 8.5 (br s, 1H), 7.2 (dd, 1H), 7.2 (d, 1H), 6.6-6.4 (m, 3H), 6.3 (m, 1H), 4.3(d, 2H), 3.9 (s, 3H), 3.8 (s, 3H).

N¹-(2,4-Dimethoxybenzyl)-5-fluorobenzene-1,2-diamine. Under a flush of Ar, a catalytic amount of a Raney Ni solution in water was added to a solution of N-(2,4-dimethoxybenzyl)-5-fluoro-2-nitrobenzenamine (0.5 g) in THF (20 mL). The flask was closed with a septum, evacuated under house vacuum and hydrogen added via balloon. The resulting suspension was stirred at RT for 16 hr, when the H₂ balloon was removed, mixture evacuated and filtered through a plug of celite, that was thoroughly rinsed with THF and MeOH, to yield the titled diamine that was used as such.

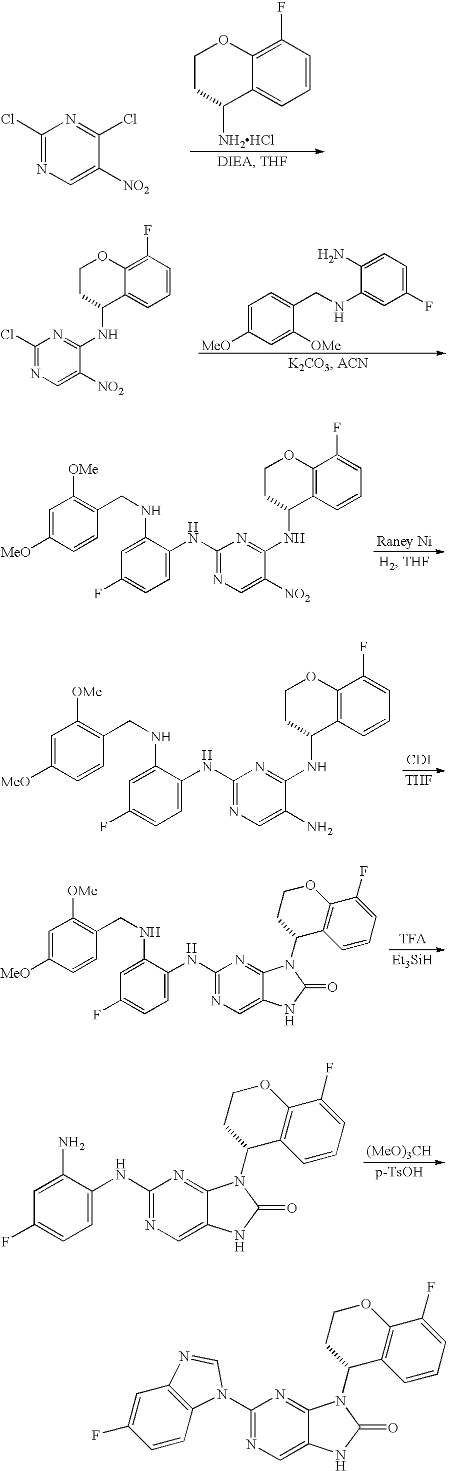

(R)-2-Chloro-N-(8-fluorochroman-4-yl)-5-nitropyrimidin-4-amine. A solution of (R)-8-fluorochroman-4-amine hydrochloride (1.02 g) and DIEA (2.6 mL) in DCM (10 mL) was slowly added to a solution of 2,4-dichloro-5-nitropyrimidine (0.97 g) THF (25 mL) at −78° C. The reaction mixture was stirred for 30 min at −78° C. then allowed to warm to RT overnight. The reaction was quenched with the addition of sat. NH₄Cl (1 mL), the solvent volume was reduced in vacuo, and the resulting mixture partition between EtOAc and water then separated. The crude material was purified via column chromatography, elution with 30% EtOAc/Hex gave the titled product (1.43 g). ¹H-NMR (300 MHz, CDCl₃) δ 9.1 (s, 1H), 8.6 (br d, 1H), 7.1-6.8 (m, 3H), 5.6 (dd,1H), 4.4 (m 1H), 4.3(m, 1H), 2.4 (m, 1H), 2.2 (m, 1H).

(R)—N²-(2-(2,4-Dimethoxybenzylamino)-4-fluorophenyl)-N⁴-(8-fluorochroman-4-yl)-5-nitropyrimidine-2,4-diamine. A mixture of (R)-2-chloro-N-(8-fluorochroman-4-yl)-5-nitropyrimidin-4-amine (32 mg), N¹-(2,4-dimethoxybenzyl)-5-fluorobenzene-1,2-diamine (28 mg)and KCO₃ (41 mg) in ACN was heated at 65° C. for 3 hr, cooled to RT, diluted with brine and extracted with EtOAc (2×). The combined organics were evaporated, and purified by column chromatography (eluted with 30% EtOAc/Hex) to yield the titled product (21 mg). ¹H-NMR (300 MHz, CDCl₃) δ 9.0 (s, 1H), 8.6 (br d, 1H), 7.2-6.8 (m, 6H), 6.5-6.3 (m,4H), 4.4-4.2 (m, 4H), 3.8 (s, 6H), 2.3-2.2 (m, 2H).

(R)-2-(2-(2,4-Dimethoxybenzylamino)-4-fluorophenylamino)-9-(8-fluorochroman-4-yl)-7H-purin-8(9H)-one. Under an Ar atmosphere, a catalytic amount of a Raney Ni solution in water was added to a solution of (R)—N²-(2-(2,4-dimethoxybenzylamino)-4-fluorophenyl)-N⁴-(8-fluorochroman-4-yl)-5-nitropyrimidine-2,4-diamine (21 mg) in THF. The flask was closed with a septum, evacuated under house vacuum and hydrogen added via balloon. The resulting suspension was stirred at RT for 2 hr, when the H₂ balloon was removed, the mixture evacuated and filtered through a plug of celite, that was thoroughly rinsed with THF and MeOH, to yield (R)—N²-(2-(2,4-dimethoxybenzylamino)-4-fluorophenyl)-N⁴-(8-fluorochroman-4-yl)pyrimidine-2,4,5-triamine that was used directly.

To a stirred solution of the above material in THF (5 mL) was added CDI (12 mg). After 18 hr brine and EtOAc were added and the mixture was separated. The organic layer was evaporated and purified by column chromatography (eluted 4% MeOH/DCM) to yield titled product (14 mg). ¹H NMR (300 MHz, CDCl₃+5% CD₃OD): δ 7.8 (s, 1H), 7.3 (s, 1H), 7.1 (d, 1H), 6.9 (m, 2H), 6.7-6.2 (m, 6H), 5.7 (dd, 1H), 4.5 (m 1H), 4.2 (m, 1H), 4.1 (s, 2H), 3.8 (s, 3H), 3.7 (s, 3H), 2.8 (m, 1H), 2.2 (m, 1H).

### 2-(5-Fluoro-1H-benzo[d]imidazol-1-yl)-9-((R)-8-fluorochroman-4-yl)-7H-purin-8(9H)-one

A mixture of (R)-2-(2-(2,4-dimethoxybenzylamino)-4-fluorophenylamino)-9-(8-fluorochroman-4-yl)-7H-purin-8(9H)-one (14 mg) and TFA (1 mL) was stirred for 60 min when triethyl silane (0.5 mL) was added. The resulting solution was stirred at RT for 16 hr, then the solvents were reduced in vacuo to yield (R)-2-(2-amino-4-fluorophenylamino)-9-(8-fluorochroman-4-yl)-7H-purin-8(9H)-one that was used as such.

A catalytic amount of p-TsOH was added to a solution of the above amine in trimethylorthoformate (2 mL). The mixture was stirred at RT for 15 hr then the solvents were reduced and the resultant material partitioned between DCM and brine and separated. The crude product was purified via column chromatography (eluted with 5% MeOH/DCM) to yield the titled compound (9 mg). ¹H-NMR (300 MHz, CDCl₃) δ 10.0 (s, 1H), 8.9 (s, 1H), 8.3 (s, 1H), 7.8 (dd, 1H), 7.4 (d, 1H), 7.1 (m, 2H), 6.8 (m, 2H), 5.9 (dd, 1H), 4.7 (m 1H), 4.4 (td, 1H), 2.9 (m, 1H), 2.4 (m, 1H).

### Example 29

### Synthesis and Resolution of 8-Fluorochroman-4-amine

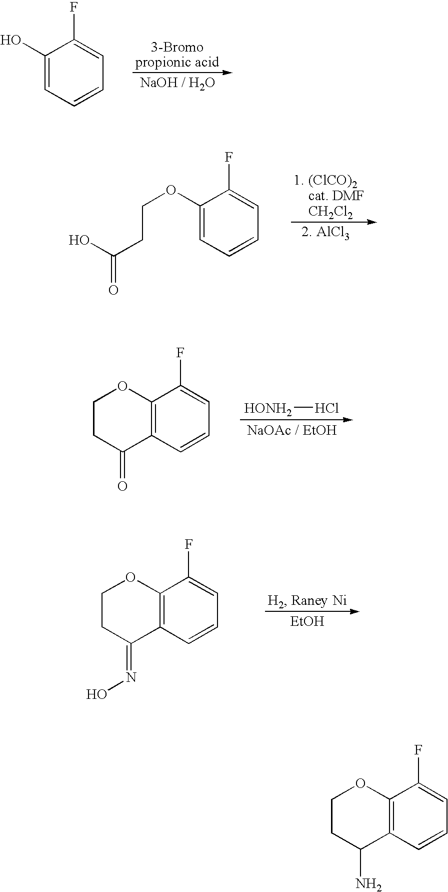

3-(2-Fluorophenoxy)propanoic acid. A mixture of 2-fluorophenol (15 g), 3-bromopropanoic acid (20 g) and NaOH (11 g) was refluxed in 50 mL of water. The solution was cooled to room temperature and acidified to pH 2 with 3 M HCl. The resulting precipitate was isolated by filtration to yield 9.27 g of title compound as a white solid. The filtrate was extracted 3 times with EtOAc to yield 2.5 g of less pure compound.

8-Fluorochroman-4-one. Oxalyl chloride (8.79 mL) and 1 drop of DMF were added to an ice cold solution of 3-(2-fluorophenoxy)propanoic acid (9.27 g) in DCM (50 mL). The solution was stirred at 0° C. for 2 hours, then aluminum chloride (7.39 g, 55.42 mM) was added and the solution was stirred for 16 hours at room temperature. The mixture was poured onto ice water, and extracted three times with DCM. The combined organics were washed with 0.5M NaOH and brine, then dried, evaporated, and purified by column chromatography (eluting with 20% EtOAc/Hex) to give of the title compound (8.20 g, 98%).

8-Fluorochroman-4-amine. A round bottom flask was charged with 8-fluorochroman-4-one (8.2 g), hydroxylamine hydrochloride (3.78 g) and sodium acetate (4.46 g). A reflux condenser was added, the flask was purged with argon, dry EtOH (20 mL) was added, and the mixture was stirred at reflux for 18 hours. The solution was cooled to room temperature, diluted with EtOAc, and washed with water. The organic phase was dried, and evaporated to give the intermediate 8-fluorochroman-4-one oxime, which was reduced with Raney Nickel in EtOH at 50 PSI to yield the titled amine (4.69 g, 57%).

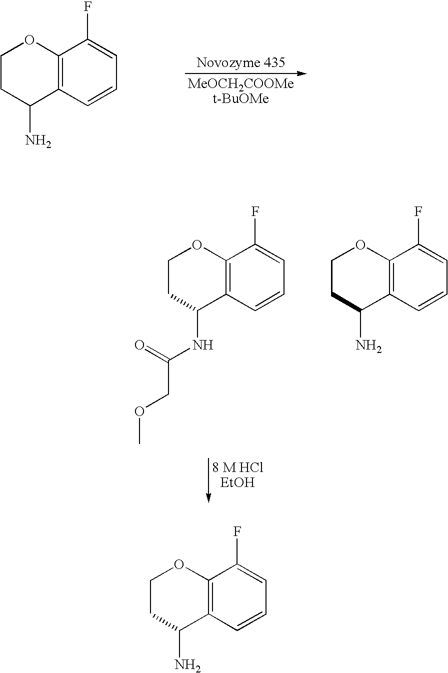

Resolution of 8-fluorochroman-4-amine. (Procedure based on US0157739). A mixture of 8-fluorochroman-4-amine (3.40 g), methyl 2-methoxyacetate (2.44 g) and Novozyme 435 (Aldrich, 0.68 g) in anhydrous tert-butyl methyl ether (75 mL) was heated at reflux under argon for 2 hours (at which time the ratio of acylated to unacylated product was 1:1 by HPLC). The solid that formed upon cooling was collected via filtration and dissolved in EtOAc. The mixture was filtered to remove the biocatalyst and washed once with 0.5M HCl to remove any lingering (S)-amine. The solvent was evaporated and the product was recrystallized from tert-butyl methyl ether to yield (R)—N-(8-fluorochroman-4-yl)-2-methoxyacetamide (0.78 g). The reaction solvent and recrystallization mother liquor was washed 3 times with 0.5 M HCl and concentrated to yield additional (R)—N-(8-fluorochroman-4-yl)-2-methoxyacetamide (0.83 g). The combined acidic aqueous layers were made basic by NaOH and extracted with DCM to yield (S)-8-fluorochroman-4-amine (1.6 g). A solution of (R)—N-(8-fluorochroman-4-yl)-2-methoxyacetamide (0.78 g) in 8M HCl in EtOH (50 mL) was heated at reflux for 4 hours. The solvents were removed from the cooled reaction mixture, the resulting solid was taken up in 50 mL of 0.5M NaOH, salted out with NaCl(s), and extracted 4 times with DCM to yield (R)-8-fluorochroman-4-amine (0.48 g (87%)). The % ee was checked via chiral HPLC: Chiralcel OD-H (0.46×25 cm analytical column, Daicel Chemical Industries) method: isocratic 5% (0.05% TFA/EtOH) 95% (0.05% TFA/Hex), Rt=7.2 min (S)-enantiomer, Rt=9.2 min (R)-enantiomer.

### Example 30

### Chroman-4-amine, 5-fluorochroman-4-amine, 6-fluorochroman-4-amine, 6-chlorochroman-4-amine, 6-methylchroman-4-amine, 6-methoxychroman-4-amine, 7-fluorochroman-4-amine, 5,8-difluorochroman-4-amine, and 6,8-difluorochroman-4-amine

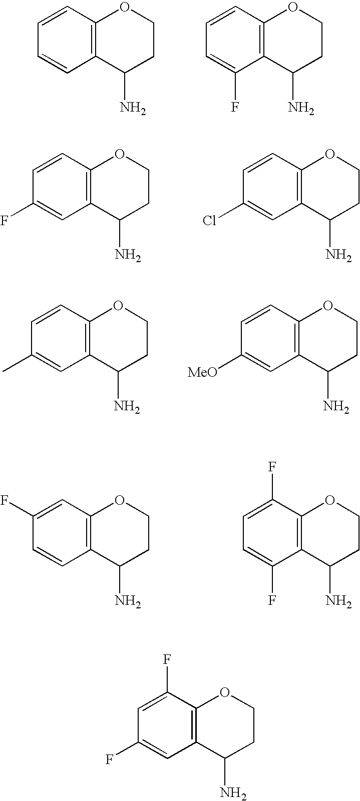

These amines were prepared via procedures described in Example 29 for the synthesis of 8-fluorochroman-4-amine. The corresponding chroman-4-ones were commercially available as advanced intermediates for the synthesis of chroman-4-amine, 6-fluorochroman-4-amine, 6-chlorochroman-4-amine, 6-methylchroman-4-amine, and 6-methoxychroman-4-amine. For the synthesis of 5-fluorochroman-4-amine, the intermediate 5-fluorochroman-4-one was obtained using procedures from GB 2355264, which also provided 7-fluorochroman-4-one. 7-Fluorochroman-4-one could be used in the synthesis of 7-fluorochroman-4-amine. Chroman-4-amine, 5-fluorochroman-4-amine, 6-fluorochroman-4-amine, 7-fluorochroman-4-amine, 5,8-difluorochroman-4-amine, and 6,8-difluorochroman-4-amine were resolved via the procedure described in Example 29 for the resolution of 8-fluorochroman-4-amine.

### Example 31

### 1-Methyl-4,5,6,7-tetrahydro-1H-indol-4-amine

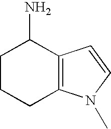

### The title compound was obtained from 1-methyl-6,7-dihydro-1H-indol-4(5H)-one (Heterocycles (1984), 22, 2313) via the procedure described in Example 29 that were used to obtain 8-fluorochroman-4-amine from 8-fluorochroman-4-one.

### Example 32

### Synthesis of 5,6-Difluorochroman-4-amine

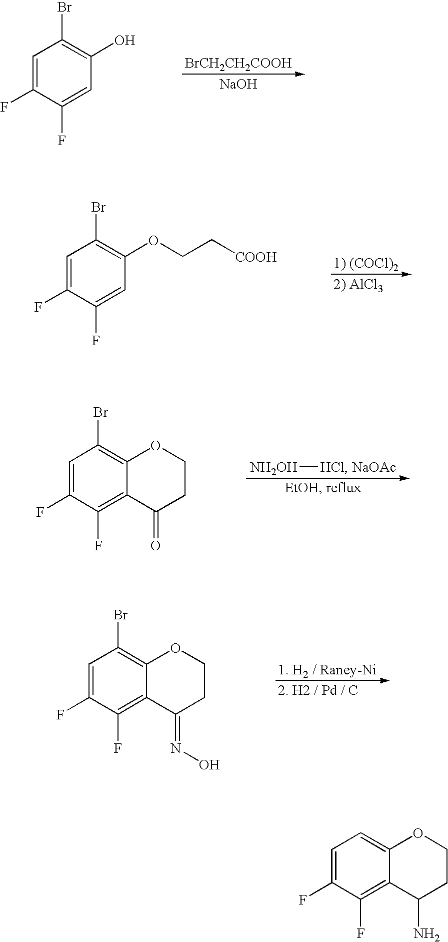

3-(2-Bromo-4,5-difluorophenoxy)propanoic acid. A solution of 1.68 g of NaOH (42 mmol) in 5 mL of water was added slowly to the suspension of 2.29 mL (20 mmol) 2-bromo-4,5-difluorophenol and 3.07 g (20 mmol) 3-bromopropionic acid. The mixture was heated at 100° C. in an oil bath for 5 hours, and then allowed to cool to room temperature. Water was added to completely dissolve any solid material and the reaction mixture was made acidic with concentrated HCl. The product was extracted into ether (3 times), and the combined organic layers was dried over Na₂SO₄ and evaporated to give 3.7 g (66%) of the title compound as a light brown solid. ¹H NMR (300 MHz, CDCl₃): δ 7.4 (t, 1H), 6.8 (q, 1H), 4.3 (t, 2H), 2.9 (t, 2H).

8-Bromo-5,6-difluoro-2,3-dihydrochromen-4-one. Oxalyl chloride (1.7 mL, 20 mmol) was added to the solution of 2.8 g (10 mmol) of 3-(2-bromo-4,5-difluorophenoxy)-propanoic acid in 40 mL of anhydrous DCM followed by a drop of DMF. After 1.5 hours, a drying tube was attached and the solution was cooled in an ice-water bath. AlCl₃ (1.5 g, 11 mmol) was added and the dark red solution was allowed to slowly reach room temperature while being stirred for 16 hours. The mixture was poured into ice and the organic layer was separated. The aqueous layer was extracted with DCM twice. The combined organic layers were washed with 0.5 N NaOH and brine, then dried over Na₂SO₄ and concentrated. Column chromatography of this residue with hexane and EtOAc provided 1.9 g of the title compound as an off-white solid (73%). ¹H NMR (300 MHz, CDCl₃): δ 7.6 (t, 1H), 4.65 (t, 2H), 2.85 (t, 2H).

8-Bromo-5,6-difluoro-2,3-dihydrochromen-4-one oxime. To a solution of 8-bromo-5,6-difluoro-2,3-dihydrochromen-4-one (7.2 mmol) in 40 mL of ethanol was added hydroxylamine hydrochloride (0.55 g, 7.9 mmol) and sodium acetate (0.65 g, 7.9 mmol). This mixture heated at reflux for 20 hrs. The mixture was cooled, diluted with EtOAc, washed with water and brine, and then dried over Na₂SO₄. Concentration of the solvent provided the title compound as a white solid (1.9 g). ¹H NMR (300 MHz, 10% CD₃OD in CDCl₃): δ 7.3 (t, 1H), 4.2 (t, 2H), 2.9 (t, 2H).

5,6-Difluoro-3,4-dihydro-2H-chromen-4-amine. Raney-Ni (5 mL slurry in water) was added to a solution of 8-bromo-5,6-difluoro-2,3-dihydrochromen-4-one oxime (1.9 g) in 200 mL MeOH. The mixture was hydrogenated at 50 psi for 24 hrs to provide 8-bromo-5,6-difluoro-3,4-dihydro-2H-chromen-4-amine. Pd/C (0.3 g) was added to the mixture and hydrogenation was resumed at 50 psi for 4 hours. The title compound was obtained after filtration and concentration in vacuo. ¹H NMR (300 MHz, 10% CD₃OD in CDCl₃): δ 7.15 (q, 1H), 6.6 (m, 1H), 4.6 (bm, 1H), 4.25 (bm, 2H), 2.2-2.4 (m, 2H).

### Example 33

### Synthesis of 4-Amino-3,4-dihydro-2H-chromene-8-carbonitrile

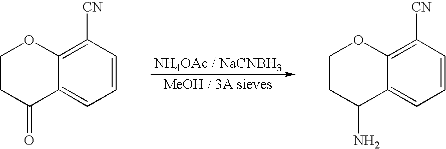

4-Amino-3,4-dihydro-2H-chromene-8-carbonitrile. A mixture of 260 mg of 4-oxo-3,4-dihydro-2H-chromene-8-carbonitrile (made from 2-hydroxybenzonitrile via the procedure described in Example 29), ammonium acetate (1.2 g), and 3A molecular sieves (1.5 g) in 10 mL of methanol was stirred for 5 days. The mixture was filtered through celite and the filtrate concentrated in vacuo. The crude residue was treated with 100 mL of 1 M HCl and extracted with ethyl ether (3×100 mL). The aqueous layer was made basic to pH 10 with saturated NaOH and extracted with DCM (3×100 mL). The combined DCM layers were washed with brine, dried over magnesium sulfate, and concentrated in vacuo to provide 150 mg of the title compound.

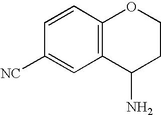

4-Amino-3,4-dihydro-2H-chromene-6-carbonitrile. The title compound was made from 6-cyano-4-chromanone (Syntech) via the same procedure that was described in Example 33.

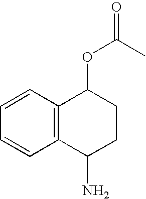

4-Amino-1,2,3,4-tetrahydronaphthalen-1-yl acetate. The title compound was made from 4-oxo-1,2,3,4-tetrahydronaphthalen-1-yl acetate (Tetrahedron: Asymmetry 2001, 12, 2283) via the same procedure that was described in Example 33.

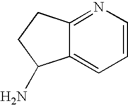

6,7-Dihydro-5H-cyclopenta[b]pyridin-5-amine. The title compound was made as described in WO 03/045924.

### Example 34

### Synthesis of (R)-5,6,7,8-Tetrahydroquinoxalin-5-amine

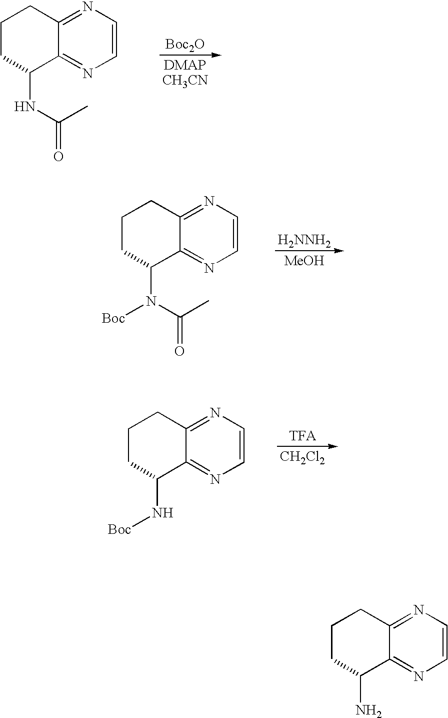

(R)-tert-Butyl acetyl(5,6,7,8-tetrahydroquinoxalin-5-yl)carbamate. A solution containing 483 mg of (R)—N-(5,6,7,8-tetrahydroquinoxalin-5-yl)acetamide (J. Org. Chem. (2003), 68, 3546) in acetonitrile (20 mL) was treated with Boc₂O (3 g) and DMAP (5 mg). The mixture was heated at 60° C. for 1.5 h and then concentrated in vacuo. Column chromatography (50% EtOAc/hexanes) provided 293 mg of the title compound.

(R)-tert-Butyl 5,6,7,8-tetrahydroquinoxalin-5-ylcarbamate. A solution of (R)-tert-butyl acetyl(5,6,7,8-tetrahydroquinoxalin-5-yl)carbamate (293 mg) in methanol (10 mL) was treated with hydrazine hydrate (0.5 mL) for 1.5 h. The mixture was diluted with EtOAc and washed twice with saturated aqueous sodium chloride. The organic layer was separated, dried with sodium sulfate, and concentrated in vacuo to provide 238 mg of the title compound.

(R)-5,6,7,8-Tetrahydroquinoxalin-5-amine. A solution of (R)-tert-butyl 5,6,7,8-tetrahydroquinoxalin-5-ylcarbamate (238 mg) in 10 ml of 1:1 TFA/DCM was stirred for 30 minutes. The mixture was concentrated in vacuo to provide the title compound as the TFA salt.

### Example 35

### Synthesis of 2-(1H-Benzo[d]imidazol-1-yl)-9-(4,5,6,7-tetrahydro-1H-indol-4-yl)-7H-purin-8(9H)-one

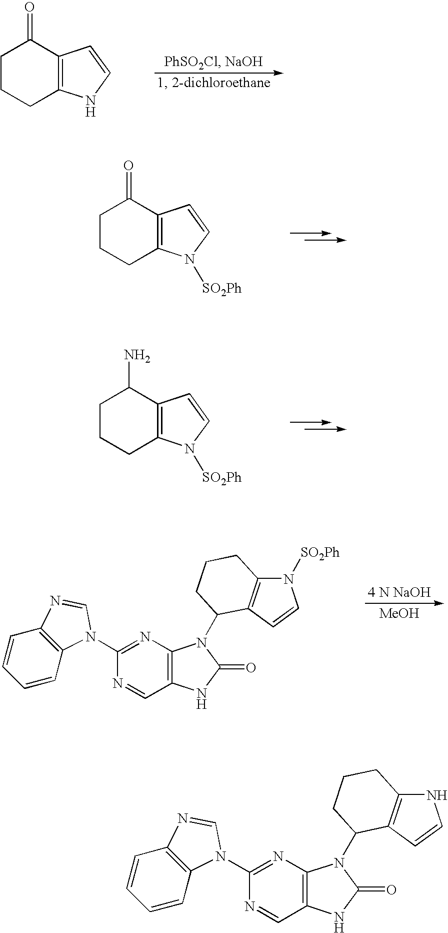

1-(Phenylsulfonyl)-4-oxo-4,5,6,7-tetrahydroindole. To a suspension of NaOH (4.44 g) in 1,2-dichloroethane (250 mL) was added 4-oxo-4,5,6,7-tetrahydroindole (5.0 g). The mixture was then cooled to 0° C. and stirred for 30 min, following which a solution of phenylsulfonyl chloride (5.7 mL) in 1,2-dichloroethane (50 mL) was added dropwise over a period of 30 min. After 30 min of stirring, the reaction mixture was allowed to come to room temperature and stirred overnight. The reaction was quenched by pouring onto distilled water (100 mL). The organic layer was separated, and the aqueous layer was extracted with dichloromethane (3×50 mL). The combined organic extract was washed with distilled water to neutrality, dried over MgSO₄, and concentrated in vacuo to afford 7.0 g of the title compound.

1-(Phenylsulfonyl)-4,5,6,7-tetrahydro-1H-indol-4-amine. The title compound was obtained from 1-(phenylsulfonyl)-4-oxo-4,5,6,7-tetrahydroindole via the procedure described in Example 29 that was used to obtain 8-fluorochroman-4-amine from 8-fluorochroman-4-one.

2-(1H-Benzo[d]imidazol-1-yl)-9-(1-(phenylsulfonyl)-4,5,6,7-tetrahydro-1H-indol-4-yl)-7H-purin-8(9H)-one. The title compound was obtained from 1-(phenylsulfonyl)-4,5,6,7-tetrahydro-1H-indol-4-amine via the procedures described in Example 24.

2-(1H-benzo[d]imidazol-1-yl)-9-(4,5,6,7-tetrahydro-1H-indol-4-yl)-7H-purin-8(9H)-one. To a solution of 2-(1H-benzo[d]imidazol-1-yl)-9-(1-(phenylsulfonyl)-4,5,6,7-tetrahydro-1H-indol-4-yl)-7H-purin-8(9H)-one (50 mg) in MeOH (1 mL) was added 4 N NaOH (1 mL), and the mixture was refluxed overnight and cooled. Volatiles were removed under reduced pressure, and the resultant neutralized with 4 N HCl. The white precipitate was filtered, washed with a small amount of water, and dried in vacuo to afford 36 mg of the title compound. ¹H NMR (d₆-DMSO) δ 11.6 (s, 1H), 10.7 (s, 1H), 8.86 (s, 1H), 8.27 (s, 1H), 7.69 (d, J=7.8 Hz, 1H), 7.58 (d, J=8.1 Hz, 1H), 7.33 (m, 2H), 6.53 (t, J=2.4 Hz, 1H), 5.64 (t, J=2.4 Hz, 1H), 5.54 (m, 1H), 2.72 (m, 2H), 2.30 (m, 1H), 2.07 (m, 2H), 1.84 (m, 1H).

### Examples 36 and 37 (Dihydrobenzofurans)

### 2-(1H-benzo[d]imidazol-1-yl)-9-(2,3-dihydrobenzofuran-3-yl)-7H-purin-8(9H)-one and 2-(1H-benzo[d]imidazol-1-yl)-9-(4-fluoro-2,3-dihydrobenzofuran-3-yl)-7H-purin-8(9H)-one

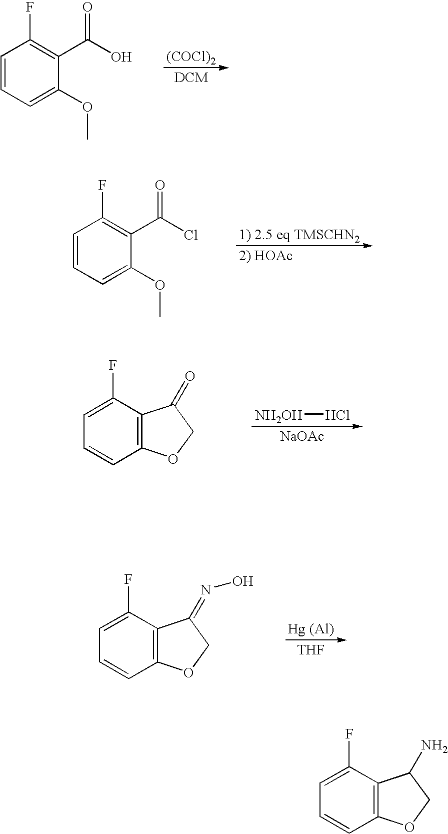

2-fluoro-6-methoxybenzoyl chloride. Oxalyl chloride (0.56 mL, 6.4 mmol) was added to the solution of 1.0 g (5.9 mmol) 2-fluoro-6-methoxybenzoic acid in 5 mL anhydrous CH₂Cl₂. Then a drop of DMF was added. After one hour, when the slow bubbling was ceased, volatiles were removed under reduced pressure to afford 1.1 g (95%) acid chloride as pale-yellow liquid.

¹H NMR (300 MHz, CDCl₃): δ 7.45 (q, 1H), 6.7-6.8 (m, 2H), 3.9 (s, 3H).

4-fluorobenzofuran-3(2H)-one. The yellow (trimethylsilyl)diazomethane ether solution (2.0 M, 3.7 mL) was added to 0.57 g (3.0 mmol) above acid chloride with stirring. After 3 hours, solvent was evaporated. The yellow residue was dissolved in 3 mL acetic acid (strong gas and heat evolution, used a water bath to cool the flask for a minute), and stirred for 15 min at room temperature. The solvents were removed under vacuum, and the red residue was taken into 2 mL CH₂Cl₂, washed with water twice, then brine, and dried over Na₂SO₄. This crude product was purified by column chromatography (eluting with 10% EtOAc in hexanes) to give 0.24 g (53%) 4-fluorobenzofuran-3(2H)-one as white solid.

¹H NMR (300 MHz, CDCl₃): δ 7.58 (m, 1H), 6.92 (br d, 1H), 6.71 (t, 1H), 4.65 (s, 2H).

(Z)-4-fluorobenzofuran-3(2H)-one oxime. The above ketone (0.70 g, 4.6 mmol) was dissolved in 5 ml ethyl alcohol, and then added 0.64 g (9.2 mmol) hydroxylamine hydrochloride and 0.75 g (9.2 mmol) sodium acetate. This suspension was brought to reflux for 1 hr. The mixture was cooled to room temperature and added 4 mL water to dissolve the excess reagents. Suction filtration, and then wash the solid cake with small amount of cold water provided 0.48 g (63%) desired oxime as white needle crystals.

¹H NMR (300 MHz, CDCl₃): δ 8.38 (s, 1H), 7.38 (q, 1H), 6.6-6.8 (m, 2H), 5.21 (s, 2H).

4-fluoro-2,3-dihydrobenzofuran-3-amine. The above oxime (0.48 g) was dissolved in 40 mL anhydrous THF under Argon. Fresh prepared aluminum amalgam (by dipping 1 g polished aluminum foil sequentially in 2% HgCl₂ aqueous solution, water, and finally THF) was added quickly and the mixture was refluxed for 24 hours under Argon. Shinny mercury beads appeared at the bottom of the flask. The mixture was allowed to cool to room temperature and filtered through a pad of celite. Flask and solid cake were washed with THF three times, then methanol three times. The combined filtrate was rotary evaporated to give 0.41 g yellow solid as a mixture of approximately (determined by NMR) 20% desired amine with 80% starting oxime. This mixture was used for next step without purification.

¹H NMR (300 MHz, CDCl₃): δ 7.18 (q, 1H), 6.5-6.7 (m, 2H), 4.8-4.9 (m, 1H), 4.69 (t, 1H), 4.2-4.3 (m, 1H).

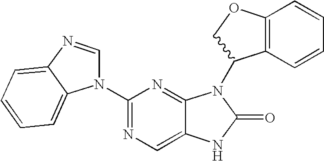

The above racemic 2-(1H-benzo[d]imidazol-1-yl)-9-(2,3-dihydrobenzofuran-3-yl)-7H-purin-8(9H)-one can be separated on chiral CHIRALCEL OD-H column (Cellulose tris(3,5-dimethylphenylcarbamate) on a 5 μM silica-gel substrate) eluting with 85:15 hexanes:ethanol (both with 0.1% diethylamine). One enantiomer has a retention time of 25 min, the other one 33.5 min.

Procedures and NMR Data for 7-Alkylated Purinone Analogs.

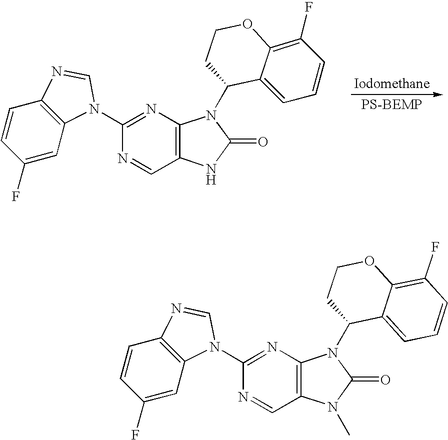

### Example 38

2-(6-fluoro-1H-benzo[d]imidazol-1-yl)-9-((R)-8-fluorochroman-4-yl)-7-methyl-7H-purin-8(9H)-one. A suspension of 2-(6-fluoro-1H-benzo[d]imidazol-1-yl)-9-((R)-8-fluorochroman-4-yl)-7H-purin-8(9H)-one (5 mg), 2-tert-butylimino-2-diethylamino-1,3-dimethyl-perhydro-1,3,2-diazaphosphorine on polystyrene (30 mg, 2.2 mM/g) and iodomethane (5 μL) in ACN (1 mL) was stirred for 1 hr at RT, then filtered through a short plug of celite, which was thoroughly rinsed with MeOH. Evaporation of the solvents and silica gel chromatography (elution with 4% MeOH/DCM) gave the titled compound (4 mg). ¹H-NMR (300 MHz, CDCl₃) δ 8.7 (s, 1H), 8.2 (s, 1H), 7.7 (m, 2H), 7.0 (m, 2H), 6.7 (m, 2H), 5.9 (dd, 1H), 4.7 (m 1H), 4.4 (td, 1H), 3.6 (s, 3H), 2.9 (m, 1H), 2.4 (m, 1H).

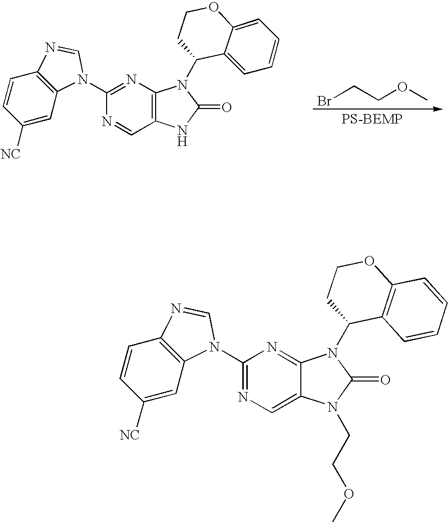

### Example 39

3-[9-Chroman-4-yl-7-(2-methoxy-ethyl)-8-oxo-8,9-dihydro-7H-purin-2-yl]-3H-benzoimidazole-5-carbonitrile. A solution of 3-(9-((R)-chroman-4-yl)-8-oxo-8,9-dihydro-7H-purin-2-yl)-3H-benzo[d]imidazole-5-carbonitrile (10 mg) in acetonitrile (10 mL). was treated with 2-bromoethylmethyl ether (23 μL) and 2-tert-butylimino-2-diethylamino-1,3-dimethyl-perhydro-1,3,2-diazaphosphorine on polystyrene (500 mg, 2.2 mmol base/g). The mixture was stirred at room temperature 16 hours, and then filtered through celite. The celite was washed with acetonitrile and methanol, and then the collected filtrate was dried with magnesium sulfate and concentrated in vacuo. Purification by column chromatography (1% MeOH/DCM) gave 4 mg of the title compound. ¹H-NMR (300 MHz, CDCl₃) δ 8.8 (s, 1H), 8.6 (s, 1H), 8.4 (s, 1H), 7.9 (d, 1H), 7.6 (d, 1H), 7.2 (m, 2H), 6.9 (d,1H), 6.8 (d, 1H), 5.9 (t, 1H), 4.6 (m, 1H), 4.4 (m, 1H), 4.2 (m, 2H), 3.8 (m, 2H), 3.4 (s, 3H), 2.8 (m, 1H), 2.4 (m, 1H).

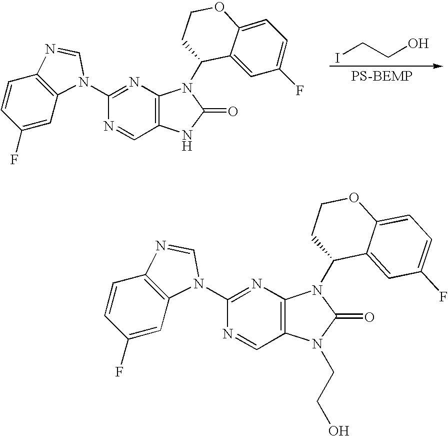

### Example 40

2-(6-fluoro-1H-benzo[d]imidazol-1-yl)-9-((R)-6-fluorochroman-4-yl)-7-(2-hydroxyethyl)-7H-purin-8(9H)-one. To the solution of 5 mg of 2-(6-fluoro-1H-benzo[d]imidazol-1-yl)-9-((R)-6-fluorochroman-4-yl)-7H-purin-8(9H)-one in 1 mL anhydrous acetonitrile was added 50 mg (excess) of 2-tert-butylimino-2-diethylamino-1,3-dimethyl-perhydro-1,3,2-diazaphosphorine on polystyrene (2.2 mmol base/g, Fluka), then 10 μL (excess) of 2-iodoethanol. The mixture was stirred at room temperature for 24 hours. Column chromatography with 3% MeOH in CH₂Cl₂ and then preparative HPLC provided 3 mg of the desired product as TFA salt. ¹H NMR (300 MHz, CDCl₃): δ 9.14 (s, 1H), 8.40 (s, 1H), 7.7-7.9 (m, 2H), 7.1-7.2 (m, 1H), 7.0-7.1 (m, 1H), 6.9-7.0 (m, 1H), 6.59 (dd, 1H), 5.88 (br t, 1H), 4.5-4.6 (m, 1H), 4.32 (t, 1H), 4.0-4.2 (m, 4H), 2.7-2.9 (bs+m, 2H), 2.2-2.3 (m, 1H).

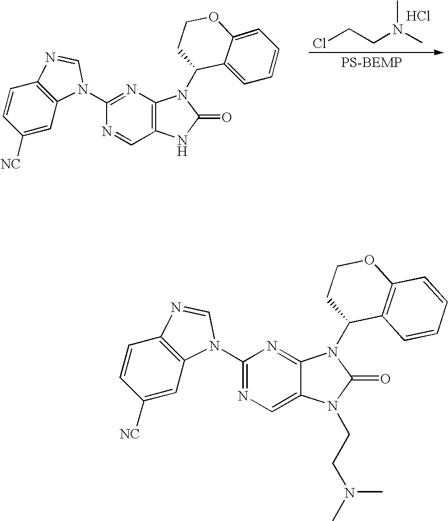

### Example 41

3-[9-Chroman-4-yl-7-(2-dimethylamino-ethyl)-8-oxo-8,9-dihydro-7H-purin-2-yl]-3H-benzoimidazole-5-carbonitrile. A solution of 3-(9-((R)-chroman-4-yl)-8-oxo-8,9-dihydro-7H-purin-2-yl)-3H-benzo[d]imidazole-5-carbonitrile (65 mg) in acetonitrile (10 mL). was treated with 2-chloro-N,N-dimethylethylamine hydrochloride (25 mg) and 2-tert-butylimino-2-diethylamino-1,3-dimethyl-perhydro-1,3,2-diazaphosphorine on polystyrene (325 mg, 2.2 mmol base/g). The mixture was stirred at room temperature for 16 hours, and then filtered through celite. The celite was washed with acetonitrile and methanol, and then the collected filtrate was dried with magnesium sulfate and concentrated in vacuo. Purification by column chromatography (2% MeOH/DCM) gave 21 mg of the title compound. ¹H-NMR (300 MHz, CDCl₃) δ 8.9 (s, 1H), 8.5 (s, 1H), 8.4 (s, 1H), 7.8 (d, 1H), 7.60 (d, 1H), 7.2 (m, 2H), 6.9 (d, 1H), 6.8 (d, 1H), 5.9 (t, 1H), 4.4 (m, 4H), 3.6 (m, 3H), 3.0 (s, 6H), 2.2 (m, 1H).

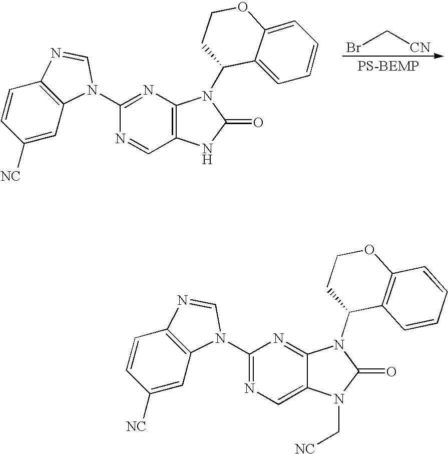

### Example 42

3-(9-Chroman-4-yl-7-cyanomethyl-8-oxo-8,9-dihydro-7H-purin-2-yl)-3H-benzoimidazole-5-carbonitrile. A solution of 3-(9-((R)-chroman-4-yl)-8-oxo-8,9-dihydro-7H-purin-2-yl)-3H-benzo[d]imidazole-5-carbonitrile (10 mg) in acetonitrile (10 mL). was treated with bromoacetonitrile (17 μL) and 2-tert-butylimino-2-diethylamino-1,3-dimethyl-perhydro-1,3,2-diazaphosphorine on polystyrene (500 mg, 2.2 mmol base/g). The mixture was stirred at room temperature 16 hours, and then filtered through celite. The celite was washed with acetonitrile and methanol, and then the collected filtrate was dried with magnesium sulfate and concentrated in vacuo. Purification by column chromatography (1% MeOH/DCM) gave 11 mg of the title compound. ¹H-NMR (300 MHz, CDCl₃) δ 8.8 (s, 1H), 8.5 (s, 1H), 8.4 (s, 1H), 7.8 (d, 1H), 7.6 (d, 1H), 7.2 (m, 2H), 6.8 (m, 2H), 5.9 (t, 1H), 5.0 (s, 2H), 4.6 (m, 1H), 4.4 (m, 1H), 2.8 (m, 1H), 2.4 (m, 1H).

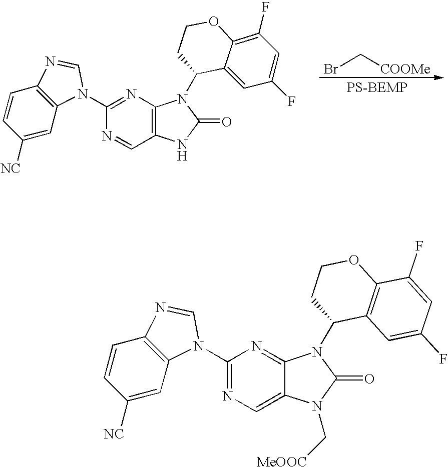

### Example 43

Methyl 2-(2-(6-cyano-1H-benzo[d]imidazol-1-yl)-9-((R)-6,8-difluorochroman-4-yl)-8-oxo-8,9-dihydropurin-7-yl)acetate. A solution of 3-(9-((R)-6,8-difluorochroman-4-yl)-8-oxo-8,9-dihydro-7H-purin-2-yl)-3H-benzo[d]imidazole-5-carbonitrile (10 mg) in acetonitrile (2 mL). was treated with methyl bromoacetate (90 mg) and 2-tert-butylimino-2-diethylamino-1,3-dimethyl-perhydro-1,3,2-diazaphosphorine on polystyrene (100 mg, 2.2 mmol base/g). The mixture was stirred at room temperature 48 hours, and then filtered. The collected filtrate was concentrated in vacuo. Purification by preparative TLC (5% MeOH/DCM) followed by column chromatography to give 6 mg of the title compound. ¹H-NMR (300 MHz, CDCl₃) δ8.94 (s, 1H), 8.78 (m, 1H), 8.21 (s, 1H), 7.91 (d, J=8.1 Hz, 1H), 7.63 (dd, J=8.1, 1.2 Hz, 1H), 6.83 (m, 1H), 6.42 (m, 1H), 5.89 (m, 1H), 4.77 (s, 2H), 4.68 (m, 1H), 4.41 (m, 1H), 3.87 (s, 3H), 2.98 (m, 1H), 2.40 (m, 1H).

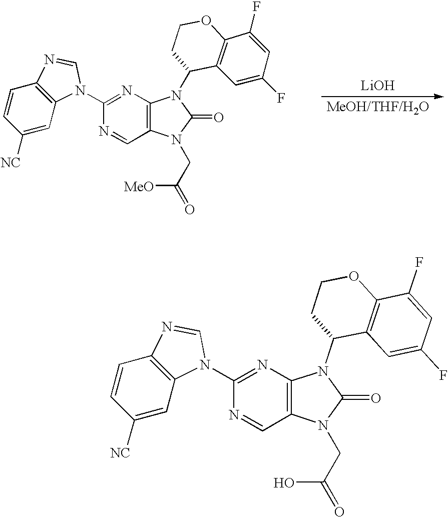

### Example 44

2-(2-(6-cyano-1H-benzo[d]imidazol-1-yl)-9-((R)-6,8-difluorochroman-4-yl)-8-oxo-8,9-dihydropurin-7-yl)acetic acid. To a solution of methyl 2-(2(6-cyano-1H-benzo[d]imidazol-1-yl)-9-((R)-6,8-difluorochroman-4-yl)-8-oxo-8,9-dihydropurin-7-yl)acetate (4 mg) in MeOH (0.75 mL) and THF (0.25 mL) at 0° C. was added a solution of lithium hydroxide (2 mg) in water (0.25 mL). The reaction was allowed to warm to room temperature and stirred for 2 h. After concentration under reduced pressure, the residue was diluted with water and acidified with 2 N HCl to pH 4. The precipitated solid was filtered, washed with a small amount of cold water, treated with 1 N HCl in MeOH (0.5 mL), and concentrated in vacuo to afford 2 mg of the title compound. ¹H-NMR (300 MHz, CDCl₃) δ9.04 (s, 1H), 8.75 (m, 1H), 8.46 (s, 1H), 7.87 (d, J=8.4 Hz, 1H), 7.68 (dd, J=8.4, 1.5 Hz, 1H), 6.92 (m, 1H), 6.70 (m, 1H), 5.97 (m, 1H), 4.77 (s, 2H), 4.63 (m, 1H), 4.44 (m, 1H), 2.91 (m, 1H), 2.42 (m, 1H).

### Example 45

### Synthesis of 2-(6-Fluoro-1H-benzo[d]imidazol-1-yl)-7-(piperidin-4-yl)-9-(tetrahydro-2H-pyran-4-yl)-7H-purin-8(9H)-one

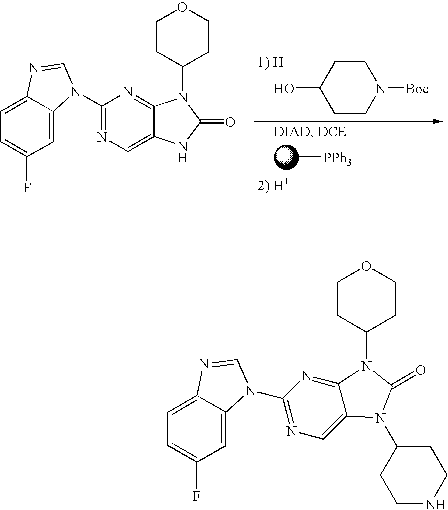

Resin bound triphenylphosphine (264 mg, 2.15 mM/g, Argonaut) and tert-butyl 4-hydroxypiperidine-1-carboxylate 6 (24 mg) were added to 2-(6-fluoro-1H-benzo[d]imidazol-1-yl)-9-(tetrahydro-2H-pyran-4-yl)-7H-purin-8(9H)-one in dichloroethane (6 mL), the mixture was stirred for 10 min when diisopropyl azodicarboxylate (64 μL) was added. After 18 hr, the material was filtered, the resins were thoroughly rinsed with ACN and MeOH, the combined organics were evaporated and purified via RP-HPLC (TFA removed Boc upon evaporation) to yield the titled compound as a TFA salt (16 mg). ¹H-NMR (300 MHz, CDCl₃+5% CD₃OD) δ 9.0 (s, 1H), 8.6 (s, 1H), 8.3 (dd, 1H), 7.7 (dd, 1H), 7.1 (td, 1H), 4.7-4.4 (m, 2H), 4.2 (dd, 2H), 3.6 (t,2H), 3.3 (br d, 2H), 2.9-2.7 (m, 4H), 2.4-2.2 (m, 4H), 2.9 (m, 1H), 1.9 (d, 2H), 1.8 (d, 2H); ¹⁹F δ −76, −117 ppm; MH⁺=438; IR (CDCl₃) 1726 (C═O stretch).

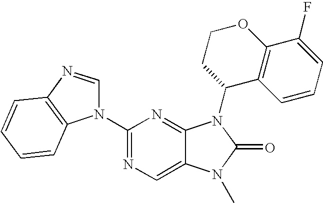

2-(1H-benzo[d]imidazol-1-yl)-9-((R)-8-fluorochroman-4-yl)-7-methyl-7H-purin-8(9H)-one. This compound was synthesized by the method described in example 38. ¹H-NMR (300 MHz, CDCl₃) δ 8.8 (s, 1H), 8.2 (s, 1H), 8.1 (m, 1H), 7.8 (m, 2H), 7.3 (m, 2H), 7.0 (m, 1H), 6.7 (m, 2H), 5.9 (t, 1H), 4.7 (m 1H), 4.4 (td, 1H), 3.6 (s, 3H), 2.9 (m, 1H), 2.4 (m, 1H).

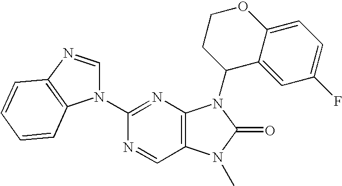

2-(1H-benzo[d]imidazol-1-yl)-9-(6-fluorochroman-4-yl)-7-methyl-7H-purin-8(9H)-one. This compound was synthesized by the method described in example 38. ¹H NMR (300 MHz, CDCl₃): δ 8.80 (s, 1H), 8.19 (s, 1H), 7.96 (d, 1H), 7.78 (d, 1H), 7.2-7.4 (m, 2H), 6.8-7.1 (m, 2H), 6.61 (dd, 1H), 5.88 (br t, 1H), 4.54 (m, 1H), 4.32 (m, 1H), 3.56 (s, 3H), 2.84 (m, 1H), 2.30 (m, 1H).

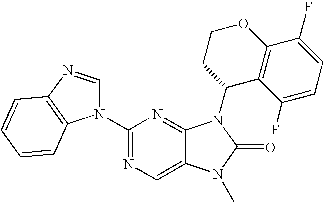

2-(1H-benzo[d]imidazol-1-yl)-9-((R)-5,8-difluorochroman-4-yl)-7-methyl-7H-purin-8(9H)-one. This compound was synthesized by the method described in example 38. ¹H NMR (300 MHz, CDCl₃): δ 8.7 (s, 1H), 8.2 (s, 1H), 8.0 (d, 1H), 7.9 (d, 1H), 7.4 (m, 2H), 7.0 (t-d, 1H), 6.5 (t-d, 1H), 6.0 (t, 1H), 4.6 (m, 1H), 4.4 (m, 1H), 3.5 (s, 3H), 2.6 (m, 1H), 2.5 (m, 1H).

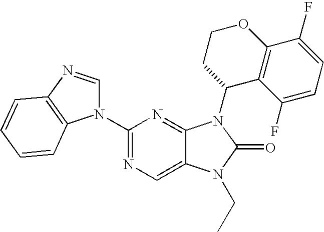

2-(1H-benzo[d]imidazol-1-yl)-9-((R)-5,8-difluorochroman-4-yl)-7-ethyl-7H-purin-8(9H)-one. This compound was synthesized by the method described in example 38 using iodoethane. ¹H NMR (300 MHz, CDCl₃): δ 8.8 (s, 1H), 8.2 (s, 1H), 8.0 (d, 1H), 7.9 (d, 1H), 7.4 (m, 2H), 7.0 (t-d, 1H), 6.5 (t-d, 1H), 6.0 (t, 1H), 4.6 (m, 1H), 4.4 (m, 1H), 4.0 (m, 2H), 2.6 (m, 1H), 2.5 (m, 1H), 1.5 (t, 3H).

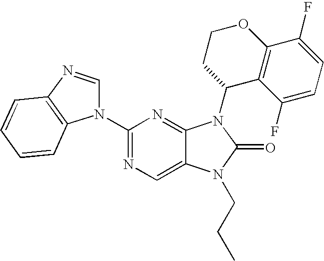

2-(1H-benzo[d]imidazol-1-yl)-9-((R)-5,8-difluorochroman-4-yl)-7-propyl-7H-purin-8(9H)-one. This compound was synthesized by the method described in example 38 using iodopropane. ¹H NMR (300 MHz, CDCl₃): δ 9.8 (s, 1H), 8.2 (s, 1H), 8.0 (d, 1H), 7.9 (d, 1H), 7.4 (m, 2H), 7.0 (t-d, 1H), 6.5 (t-d, 1H), 6.0 (t, 1H), 4.6 (m, 1H), 4.4 (m, 1H), 4.0 (m, 2H), 2.6 (m, 1H), 2.5 (m, 1H), 1.9 (m, 2H), 1.0 (t, 3H).

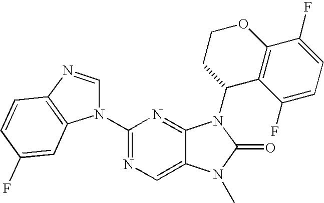

9-((R)-5,8-difluorochroman-4-yl)-2-(6-fluoro-1H-benzo[d]imidazol-1-yl)-7-methyl-7H-purin-8(9H)-one. This compound was synthesized by the method described in example 38. ¹H-NMR (300 MHz, CDCl₃) δ 8.7 (s, 1H), 8.0 (m, 1H), 7.7 (dd, 1H), 7.1 (m, 2H), 6.5 (td, 1H), 5.9 (dd, 1H), 4.6 (m 1H), 4.5 (m, 1H), 3.5 (s, 3H), 2.6 (m, 1H), 2.5 (m, 1H).

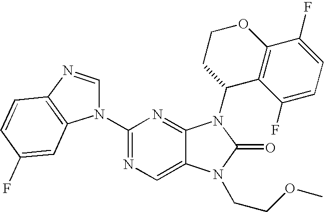

9-((R)-5,8-difluorochroman-4-yl)-2-(6-fluoro-1H-benzo[d]imidazol-1-yl)-7-(2-methoxyethyl)-7H-purin-8(9H)-one. This compound was synthesized by the method described in example 39. ¹H-NMR (300 MHz, CDCl₃) δ 8.7 (s, 1H), 8.4 (s, 1H), 8.0 (m, 1H), 7.8 (dd, 1H), 7.2-7.0 (m, 2H), 6.5 (td, 1H), 6.0 (t, 1H), 4.6 (m 1H), 4.5 (m, 1H), 4.1 (t, 2H), 3.7 (t, 2H), 3.4 (s, 3H), 2.7 (m, 1H), 2.4 (m, 1H).

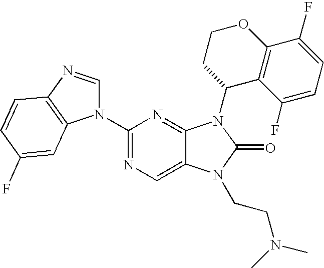

9-((R)-5,8-difluorochroman-4-yl)-7-(2-(dimethylamino)ethyl)-2-(6-fluoro-1H-benzo[d]imidazol-1-yl)-7H-purin-8(9H)-one. This compound was synthesized by the method described in example 41. ¹H-NMR (300 MHz, CDCl₃) δ 9.2 (s, 1H), 8.5 (s, 1H), 7.9 (m, 1H), 7.8 (dd, 1H), 7.2 (td, 1H), 7.1 (td, 1H), 6.5 (td, 1H), 5.9 (t, 1H), 4.6 (m 1H), 4.5 (m, 3H), 3.6 (m, 2H), 3.0 (s, 6H), 2.7 (m, 1H), 2.5 (m, 1H).

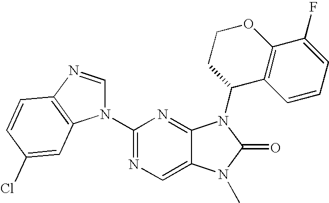

2-(6-Chloro-1H-benzo[d]imidazol-1-yl)-9-((R)-8-fluorochroman-4-yl)-7-methyl-7H-purin-8(9H)-one. This compound was synthesized by the method described in example 38. ¹H-NMR (300 MHz, CDCl₃) δ 8.7 (s, 1H), 8.3 (m, 1H), 8.2 (s, 1H), 7.7 (d, 1H), 7.3 (dd, 1H), 7.0 (td, 1H), 6.8-6.6 (m, 2H), 5.9 (dd, 1H), 4.7 (m 1H), 4.5 (td, 1H), 3.6 (s, 3H), 3.0 (m, 1H), 2.4 (m, 1H).

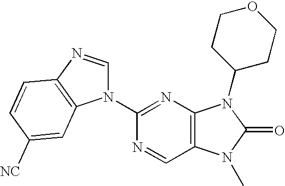

3-(7-methyl-8-oxo-9-(tetrahydro-2H-pyran-4-yl)-8,9-dihydro-7H-purin-2-yl)-3H-benzo[d]imidazole-5-carbonitrile. This compound was synthesized by the method described in example 38. ¹H-NMR (300 MHz, CDCl₃) δ 9.37 (s, 1H), 8.90 (m, 1H), 8.58 (s, 1H), 8.00 (d, J=8.4 Hz, 1H), 7.79 (dd, J=8.4, 1.5 Hz, 1H), 4.63 (m, 1H), 4.03 (m, 2H), 3.51 (m, 2H), 3.44 (s, 3H), 2.58 (m, 2H), 1.80 (m, 2H).

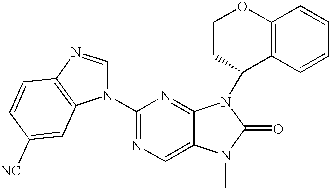

3-(9-Chroman-4-yl-7-methyl-8-oxo-8,9-dihydro-7H-purin-2-yl)-3H-benzoimidazole-5-carbonitrile. This compound was synthesized by the method described in example 38. ¹H-NMR (300 MHz, CDCl₃) δ 8.8 (s, 1H), 8.5 (s, 1H), 8.2 (s, 1H), 7.8 (d, 1H), 7.6 (d, 1H), 7.2 (m, 2H), 6.9 (d, 1H), 6.8 (d, 1H), 5.9 (t, 1H), 4.6 (m, 1H), 4.4(m, 1H), 3.6 (s, 3H), 2.8 (m, 1H), 2.4 (m, 1H).

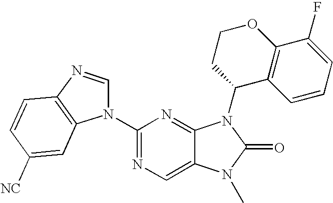

3-[9-(8-Fluoro-chroman-4-yl)-7-methyl-8-oxo-8,9-dihydro-7H-purin-2-yl]-3H-benzoimidazole-5-carbonitrile. This compound was synthesized by the method described in example 38. ¹H-NMR (300 MHz, CDCl₃) δ 8.8 (d, 2H), 8.2 (s, 1H), 7.8 (d, 1H), 7.6 (d, 1H), 7.0 (t, 1H), 6.6 (m, 2H), 5.9 (t, 1H), 4.6 (m, 1H), 4.4 (m, 1H), 3.6 (s, 3H), 2.8 (m, 1H), 2.4 (m, 1H).

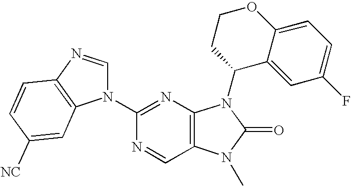

3-(9-((R)-6-fluorochroman-4-yl)-7-methyl-8-oxo-8,9-dihydro-7H-purin-2-yl)-3H-benzo[d]imidazole-5-carbonitrile. This compound was synthesized by the method described in example 38. ¹H NMR (300 MHz, CDCl₃): δ 8.90 (s, 1H), 8.52 (s, 1H), 8.21 (s, 1H), 7.84 (d, 1H), 7.59 (d, 1H), 7.1-7.2 (m, 1H), 6.9-7.0 (m, 1H), 6.59 (dd, 1H), 5.89 (br t, 1H), 4.5-4.6 (m, 1H), 4.34 (br t, 1H), 3.59 (s, 3H), 2.8-2.9 (m, 1H), 2.3-2.4 (m, 1H).

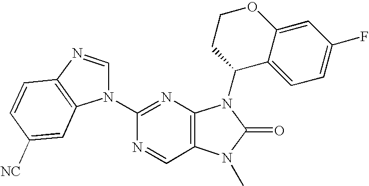

3-(9-((R)-7-fluorochroman-4-yl)-7-methyl-8-oxo-8,9-dihydro-7H-purin-2-yl)-3H-benzo[d]imidazole-5-carbonitrile. This compound was synthesized by the method described in example 38. ¹H NMR (300 MHz, CDCl₃): δ 8.90 (s, 1H), 8.62 (s, 1H), 8.19 (s, 1H), 7.84 (d, 1H), 7.59 (d, 1H), 6.8-7.0 (m, 2H), 6.4-6.6 (m, 1H), 5.86 (br t, 1H), 4.5-4.6 (m, 1H), 4.36 (br t, 1H), 3.59 (s, 3H), 2.8-2.9 (m, 1H), 2.3-2.4 (m, 1H).

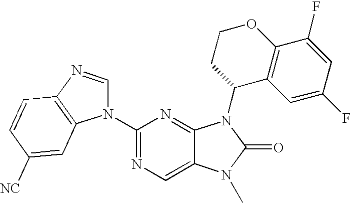

3-(9-((R)-6,8-difluorochroman-4-yl)-7-methyl-8-oxo-8,9-dihydro-7H-purin-2-yl)-3H-benzo[d]imidazole-5-carbonitrile. This compound was synthesized by the method described in example 38. ¹H-NMR (300 MHz, CDCl₃) δ 8.9 (s, 1H), 8.8 (s, 1H), 8.2 (s, 1H), 7.9 (d, 1H), 7.6 (dd, 1H), 6.8 (td, 1H), 6.4 (dd, 1H), 5.9 (dd, 1H), 4.7 (m, 1H), 4.4 (td, 1H), 3.6 (s, 3H), 3.0 (m, 1H), 2.4 (m, 1H).

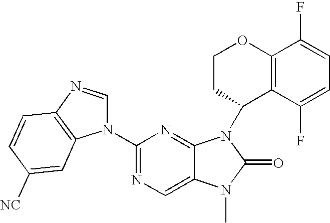

3-[9-(5,8-Difluoro-chroman-4-yl)-7-methyl-8-oxo-8,9-dihydro-7H-purin-2-yl]-3H-benzoimidazole-5-carbonitrile. This compound was synthesized by the method described in example 38. ¹H-NMR (300 MHz, CDCl₃) δ 8.8 (s, 1H), 8.7 (s, 1H), 8.2 (s, 1H), 7.8 (d, 1H), 7.6 (d, 1H), 7.0 (m, 1H), 6.5 (m, 1H), 5.9 (t, 1H), 4.5 (m, 2H), 3.5 (s, 3H), 2.5(m, 2H).

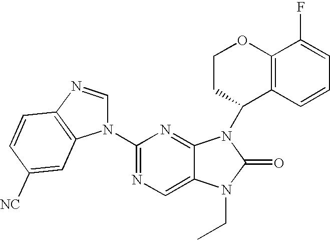

3-[7-Ethyl-9-(8-fluoro-chroman-4-yl)-8-oxo-8,9-dihydro-7H-purin-2-yl]-3H-benzoimidazole-5-carbonitrile. This compound was synthesized by the method described in example 38 using iodoethane. ¹H-NMR (300 MHz, CDCl₃) δ 8.8 (s, 2H), 8.2 (s, 1H), 7.9 (d, 1H), 7.6 (d, 1H), 7.0 (t, 1H), 6.7 (m, 2H), 5.9 (t, 1H), 4.6 (m, 1H), 4.4(m, 1H), 4.0 (q, 2H), 2.9 (m, 1H), 2.4 (m, 1H), 1.4 (t, 3H).

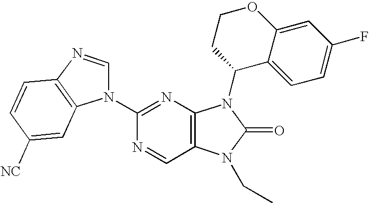

3-(7-ethyl-9-((R)-7-fluorochroman-4-yl)-8-oxo-8,9-dihydro-7H-purin-2-yl)-3H-benzo[d]imidazole-5-carbonitrile. This compound was synthesized by the method described in example 38 using iodoethane. ¹H NMR (300 MHz, CDCl₃): δ 8.88 (s, 1H), 8.62 (s, 1H), 8.21 (s, 1H), 7.84 (d, 1H), 7.59 (d, 1H), 6.8-7.0 (m, 2H), 6.4-6.6 (m, 1H), 5.86 (br t, 1H), 4.5-4.6 (m, 1H), 4.36 (br t, 1H), 4.0-4.1 (m, 2H), 2.8-2.9 (m, 1H), 2.3-2.4 (m, 1H), 1.48 (t, 3H).

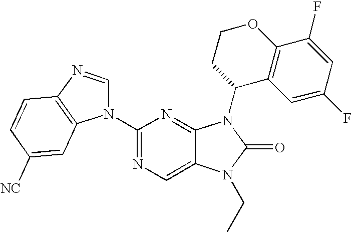

3-(9-((R)-6,8-difluorochroman-4-yl)-7-ethyl-8-oxo-8,9-dihydro-7H-purin-2-yl)-3H-benzo[d]imidazole-5-carbonitrile. This compound was synthesized by the method described in example 38 using iodoethane. ¹H-NMR (300 MHz, CDCl₃) δ 8.9 (s, 1H), 8.8 (s, 1H), 8.2 (s, 1H), 7.9 (d, 1H), 7.6 (dd, 1H), 6.8 (td, 1H), 6.4 (dd, 1H), 5.9 (dd, 1H), 4.7 (m, 1H), 4.4 (td, 1H), 4.1 (q, 2H), 3.0 (m, 1H), 2.4 (m, 1H), 1.5 (t, 3H).

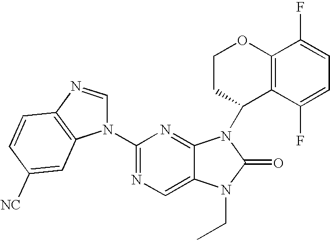

3-[9-(5,8-Difluoro-chroman-4-yl)-7-ethyl-8-oxo-8,9-dihydro-7H-purin-2-yl]-3H-benzoimidazole-5-carbonitrile. This compound was synthesized by the method described in example 38 using iodoethane. ¹H-NMR (300 MHz, CDCl₃) δ 8.8 (s, 1H), 8.7 (s, 1H), 8.2 (s, 1H), 7.9 (d, 1H), 7.6 (d, 1H), 7.0 (m, 1H), 6.4 (m, 1H), 5.9 (t, 1H), 4.6 (m, 1H), 4.4(m, 1H), 4.0 (q, 2H), 2.5(m, 2H), 1.4 (t, 3H).

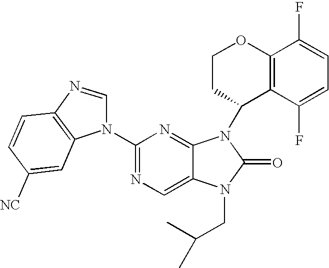

3-[9-(5,8-Difluoro-chroman-4-yl)-7-isobutyl-8-oxo-8,9-dihydro-7H-purin-2-yl]-3H-benzoimidazole-5-carbonitrile. This compound was synthesized by the method described in example 38 using iso-butyl iodide. ¹H-NMR (300 MHz, CDCl₃) δ 8.9 (s, 1H), 8.8 (s, 1H), 8.2 (s, 1H), 7.9 (d, 1H), 7.6 (d, 1H), 7.1 (m, 1H), 6.5 (m, 1H), 5.9 (t, 1H), 4.6 (m, 1H), 4.4(m, 1H), 3.8 (q, 2H), 2.7 (m, 1H), 2.5 (m, 1H), 2.3 (q, 1H), 1.0 (s, 6H).

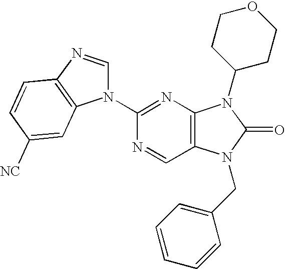

3-(7-benzyl-8-oxo-9-(tetrahydro-2H-pyran-4-yl)-8,9-dihydro-7H-purin-2-yl)-3H-benzo[d]imidazole-5-carbonitrile. This compound was synthesized by the method described in example 38 using benzyl bromide. ¹H NMR (300 MHz, CDCl₃): δ 9.30 (s, 1H), 9.01 (m, 1H), 8.23 (s, 1H), 7.89 (m, 1H), 7.66 (dd, J=8.1, 1.5 Hz, 1H), 7.40-7.32 (m, 5H), 5.16 (s, 2H), 4.75 (m, 1H), 4.18 (m, 2H), 3.63 (m, 2H), 2.81 (m, 2H), 1.90 (m, 2H).

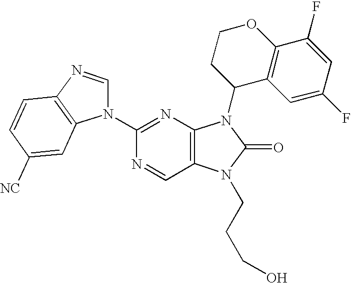

3-(9-(6,8-difluorochroman-4-yl)-7-(3-hydroxypropyl)-8-oxo-8,9-dihydro-7H-purin-2-yl)-3H-benzo[d]imidazole-5-carbonitrile. This compound was synthesized by the method described in example 40 using 3-iodopropanol. ¹H NMR (300 MHz, CDCl₃): δ 8.92 (s, 1H), 8.77 (m, 1H), 8.38 (s, 1H), 7.90 (d, J=8.4 Hz, 1H), 7.62 (dd, J=8.4, 1.5 Hz, 1H), 6.83 (m, 1H), 6.38 (m, 1H), 5.88 (m, 1H), 4.67 (m, 1H), 4.41 (m, 1H), 4.19 (t, J=6.5 Hz, 2H), 3.74 (t, J=5.6 Hz, 2H), 2.93 (m, 1H), 2.40 (m, 1H), 2.08 (m, 2H).

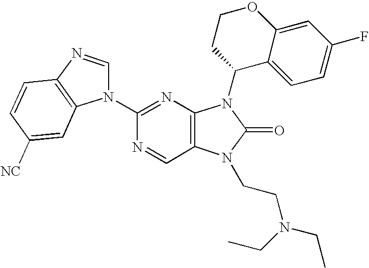

3-(7-(2-(diethylamino)ethyl)-9-((R)-7-fluorochroman-4-yl)-8-oxo-8,9-dihydro-7H-purin-2-yl)-3H-benzo[d]imidazole-5-carbonitrile. This compound was synthesized by the method described in example 41 using 2-chloro-N,N-diethylethylamine hydrochloride. ¹H NMR (300 MHz, CDCl₃): δ 8.91 (s, 1H), 8.64 (s, 1H), 8.28 (s, 1H), 7.84 (d, 1H), 7.59 (d, 1H), 6.8-7.0 (m, 2H), 6.4-6.6 (m, 1H), 5.86 (br t, 1H), 4.5-4.7 (m, 1H), 4.3-4.4 (m, 1H), 4.0-4.1 (m, 2H), 2.8-3.0 (m, 3H), 2.58 (q, 4H), 2.3-2.4 (m, 1H), 0.96 (t, 6H).

### Jak3 Kinase Assay

Human Jak3 cDNA was amplified by PCR. A fragment encoding the catalytic domain of Jak3 (508aa to 1124aa) was ligated with GST at 5′ end. This fused GST-Jak3 DNA fragment was cloned into the EcoRI site of the donor plasmid pFastBac 1 (Life Technologies #10359-016). The transformation, transposition, and transfection of insect cells (Sf9) were performed according to the manufacture's instructions. The cell lysate containing recombinant GST-Jak3 was used in the kinase assay. Anti-GST antibody (10 μg/ml, Sigma #G1417) was coated onto a 384-well plate at 4° C. overnight. Cell lysate containing GST-Jak3 (1:100 dilution) was added to the anti-GST coated plates, and GST-Jak3 was captured by immobilized anti-GST antibody. Testing compounds and substrate mix (50 mM HEPES, pH 7, 0.5 mM Na₃VO₄, 25 mM MgCl₂, 1 mM DTT, 0.005% BSA, 1 μM ATP, and 4.5 μg/ml biotinyl poly-Glu,Ala,Tyr) were added to the plate to initiate the reaction. After a 60-min incubation, the reaction was stopped by 4 mM EDTA, and phosphorylation of biotinyl poly-Glu,Ala,Tyr was detected using 17 μg/ml Cy5-streptavidin (Amersham, #PA92005) and 2.7 μg/ml Europium-conjugated anti-phosphotyrosine antibody (PerkinElmer #AD0069) using homogeneous time-resolved fluorescence (HTRF) technology.

### Jak3 Cellular Assay

The mouse F7 pre-B lymphocyte cell line was used for the cellular Jak3 assay. Human IL-2βc cDNA is stably expressed in F7 cells (Kawahara et al., 1995). F7 cells were maintained in RPMI 1640 medium supplemented with 10% fetal bovine serum plus IL-3. Cells (30,000 cells/well) in serum-free medium were seeded in 96-well plates for the cell proliferation assay. Testing compounds were added to cells, followed by the addition of IL-2 (final 20 ng/ml). After a 24-h incubation, the number of viable cells was determined by the CellTiter-Glo Luminescent Cell Viability Assay kit (Promega, #G7573) according to the manufacturer's instructions.

The results of testing of representative species are shown below. The compounds in Table 1 exhibited IC₅₀ less than 100 nM. The compounds in Table 2 exhibited IC₅₀ between 101 nM and 1 μM.

TABLE 1

| Synthesis Example | Reference No. | |
|---|---|---|
| | 501 | 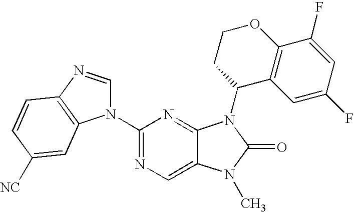 |
| | 502 | 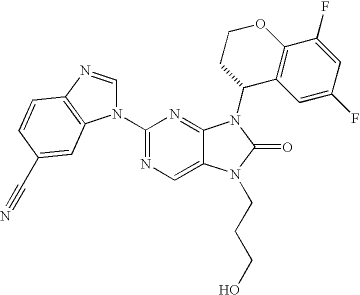 |
| Example 42 | 503 | 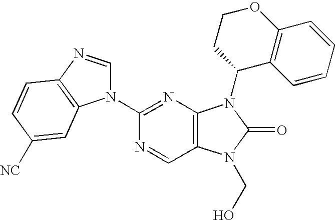 |
| | 504 | 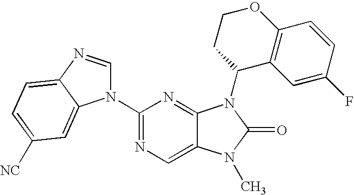 |
| | 505 | 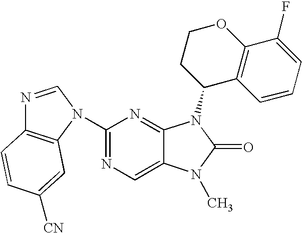 |
| | 506 | 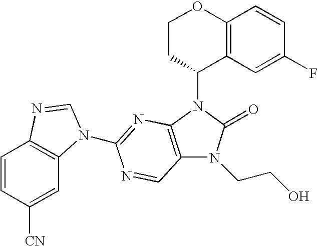 |
| | 507 | 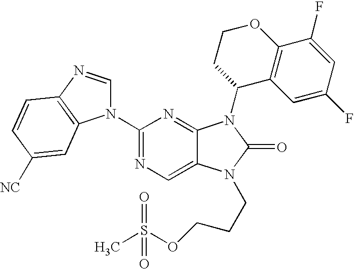 |
| | 508 | 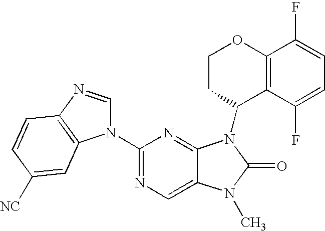 |
| | 509 | 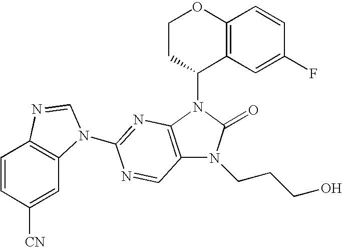 |
| | 510 | 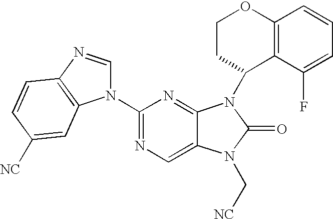 |
| | 511 | 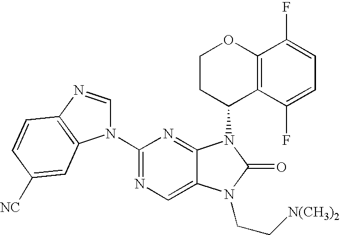 |
| | 512 | 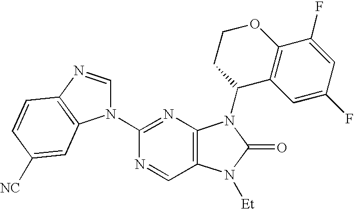 |
| Example 44 | 513 | 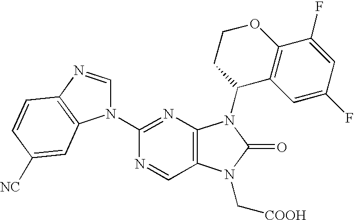 |
| | 514 | 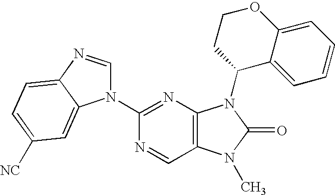 |
| Example 43 | 515 | 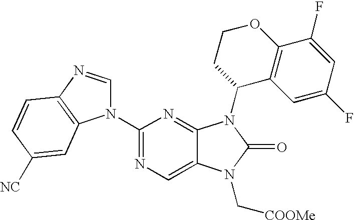 |
| | 516 | 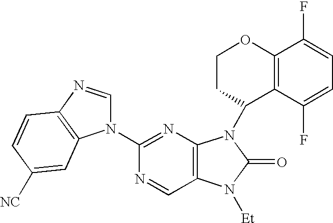 |
| | 517 | 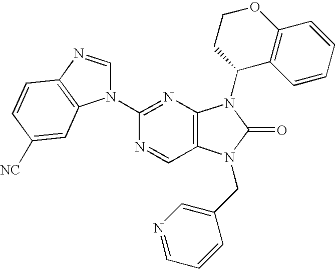 |
| | 518 | 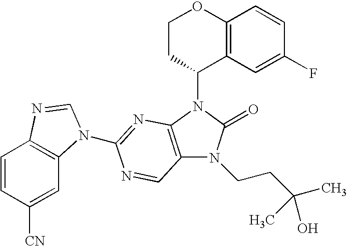 |
| | 519 | 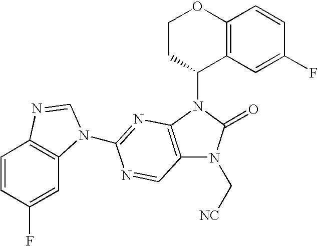 |
| | 520 | 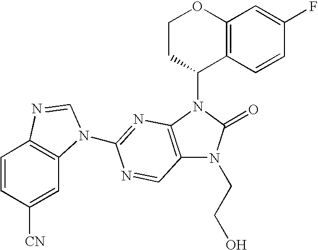 |
| | 521 | 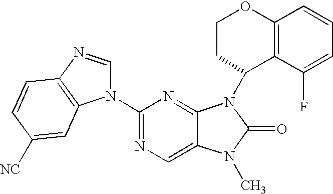 |
| Example 40 | 522 | 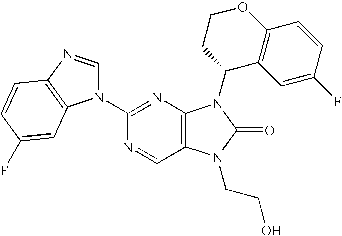 |
| | 523 | 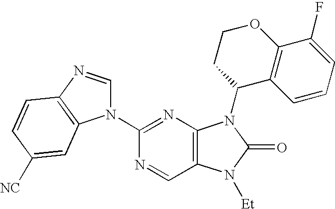 |
| Example 39 | 524 | 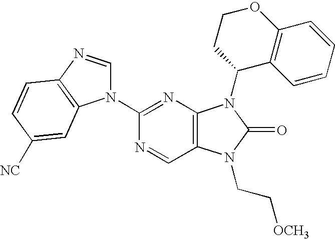 |
| | 525 | 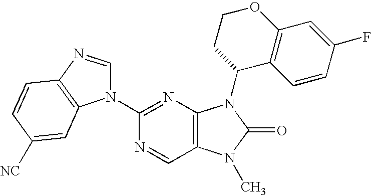 |
| Example 41 | 526 | 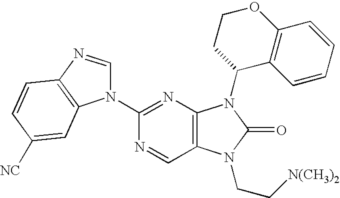 |
| | 527 | 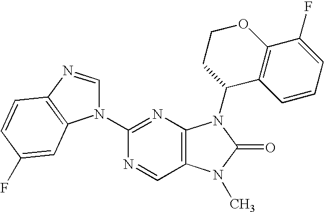 |
| | 528 | 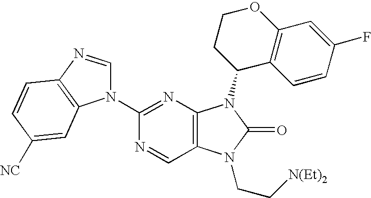 |
| | 529 | 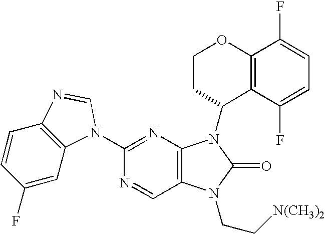 |
| | 530 | 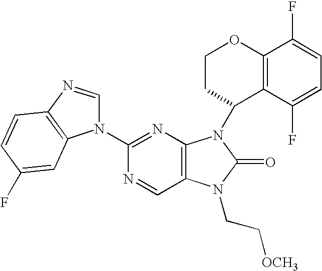 |
| | 531 | 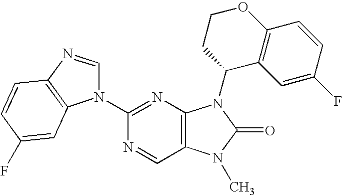 |
| | 532 | 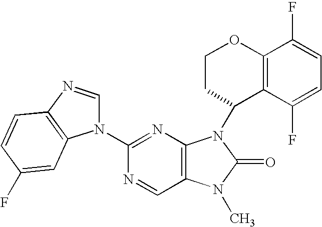 |
| | 533 | 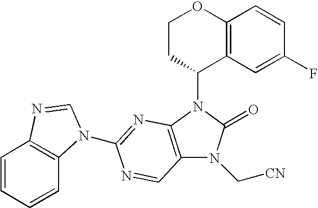 |
| | 534 | 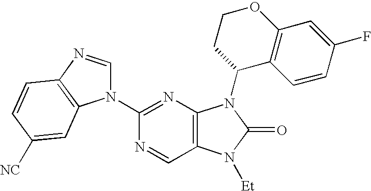 |
| | 535 | 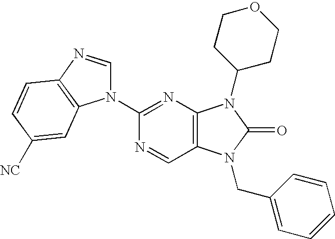 |
| | 536 | 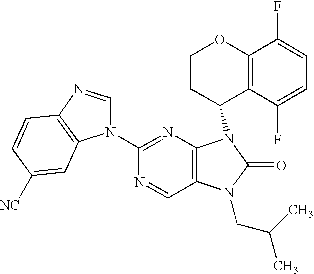 |
| | 537 | 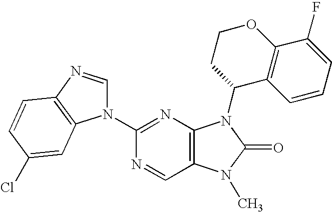 |
| | 538 | 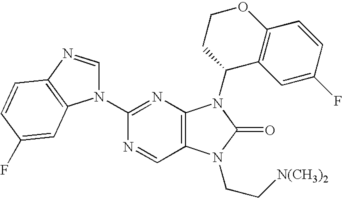 |
| | 539 | 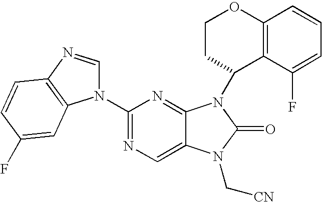 |
| Example | 540 | 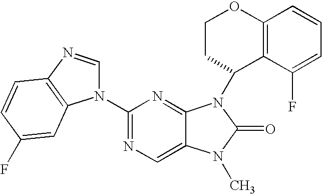 |
| | 541 | 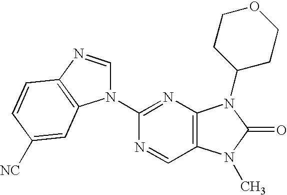 |
| Example | 542 | 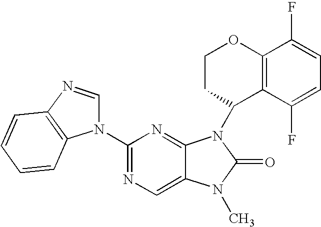 |
| | 543 | 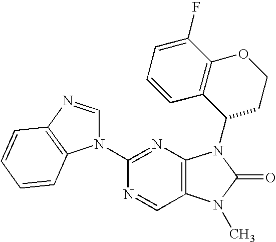 |
| | 544 | 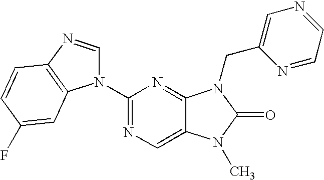 |
| | 545 | 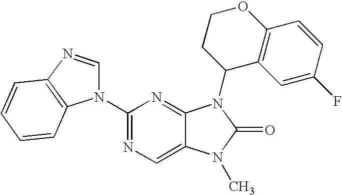 |
| | 546 | 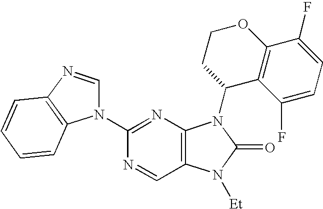 |
| | 701 | 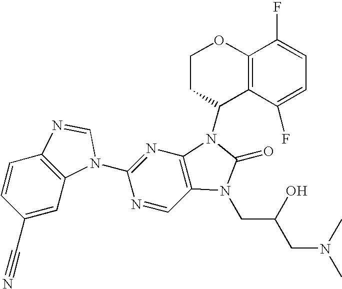 |
| | 702 | 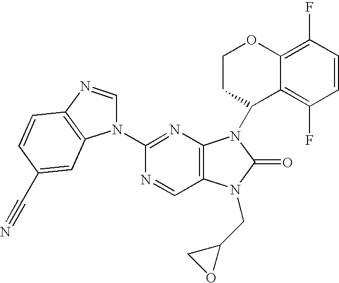 |
| | 703 | 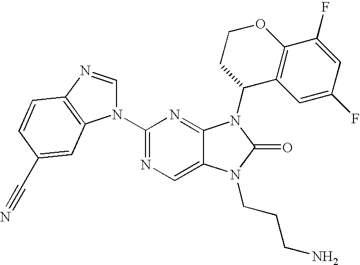 |
| | 704 | 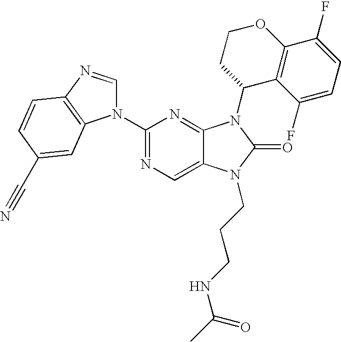 |
| | 705 | 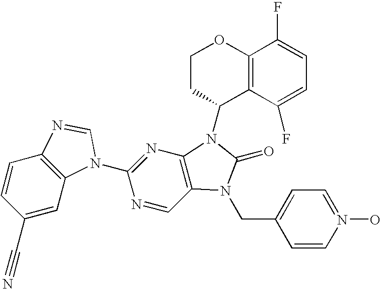 |
| | 706 | 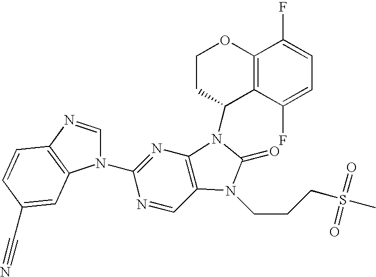 |
| | 707 | 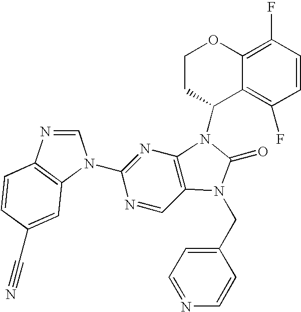 |
| | 708 | 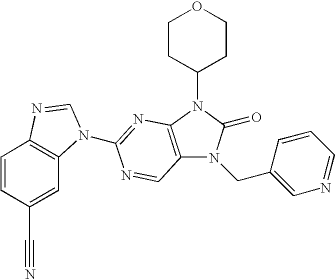 |
| | 709 | 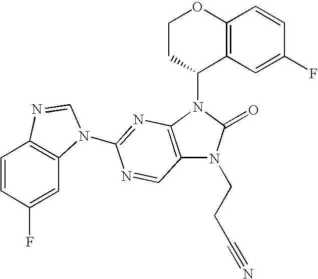 |
| | 710 | 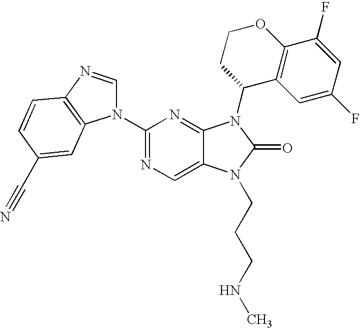 |
| | 711 | 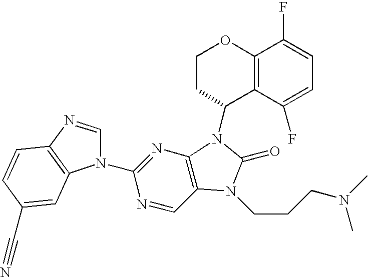 |
| | 712 | 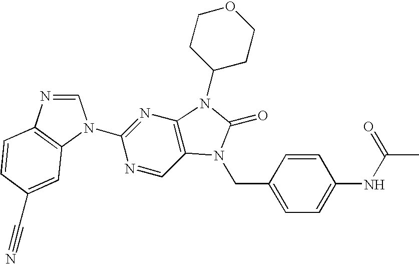 |
| | 713 | 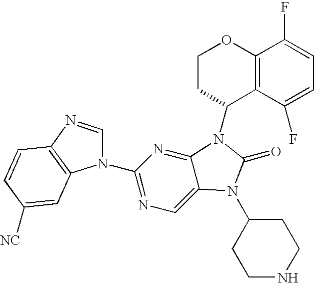 |
| | 714 | 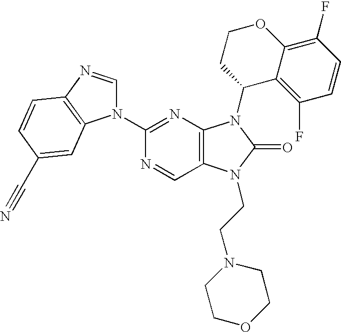 |
| | 715 | 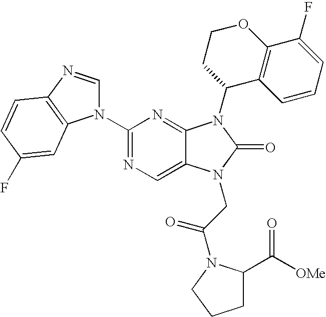 |
| | 716 | 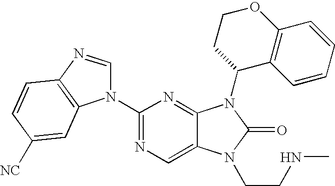 |
| | 717 | 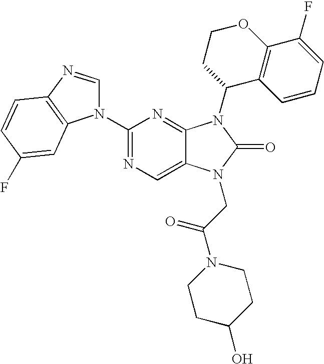 |
| | 718 | 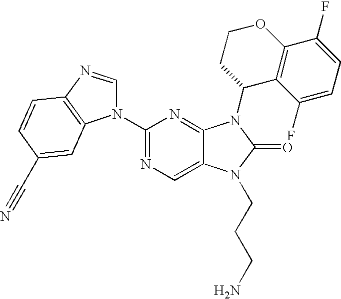 |
| | 719 | 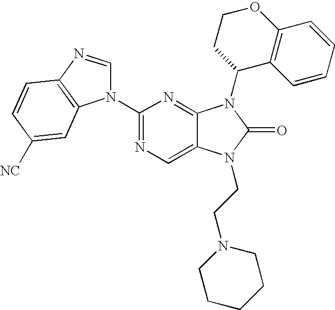 |
| | 710 | 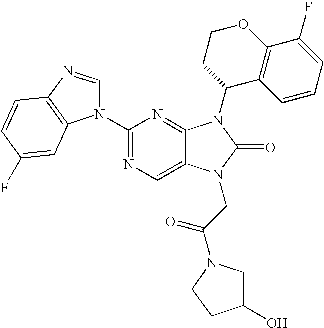 |
| | 711 | 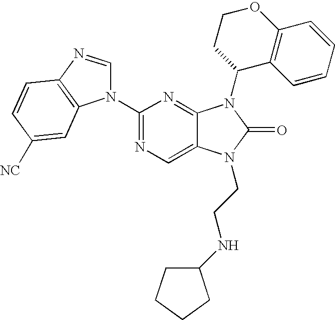 |
| | 712 | 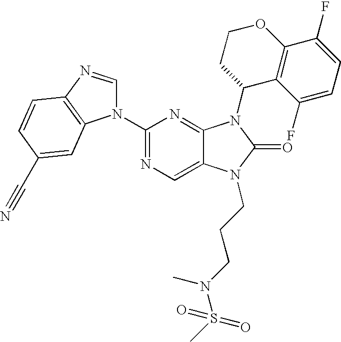 |
| | 713 | 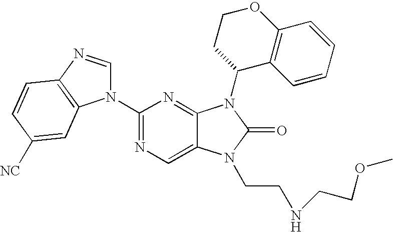 |
| | 714 | 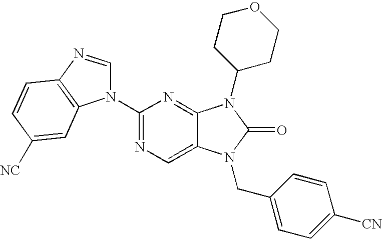 |
| | 715 | 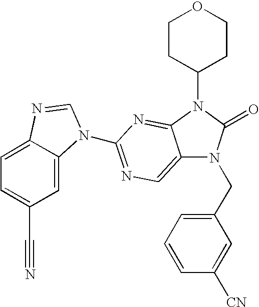 |
| | 716 | 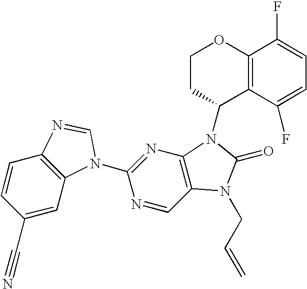 |
| | 717 | 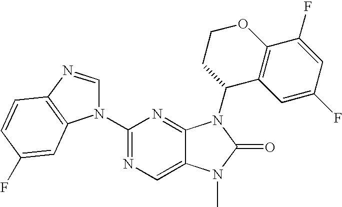 |
| | 718 | 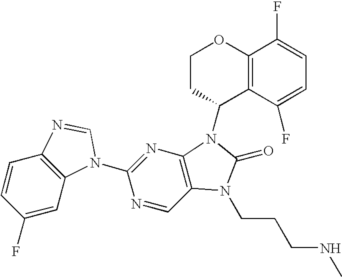 |
| | 719 | 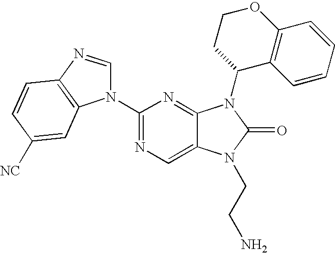 |
| | 720 | 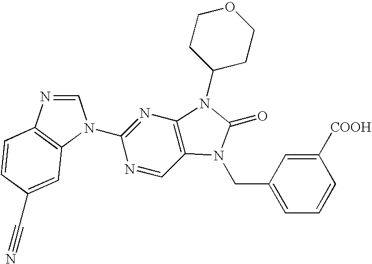 |
| | 721 | 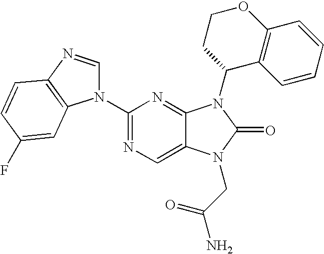 |
| | 722 | 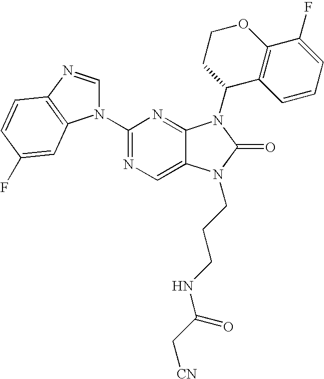 |
| | 723 | 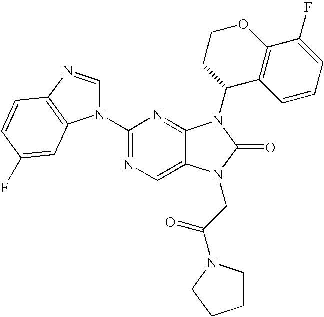 |
| | 724 | 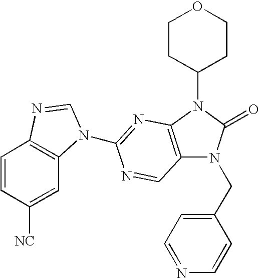 |
| | 725 | 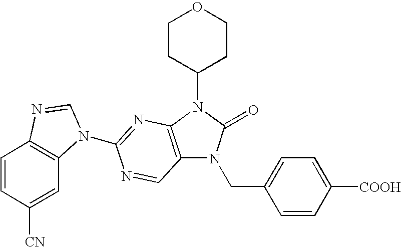 |
| | 726 | 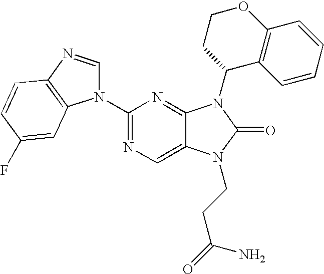 |
| | 727 | 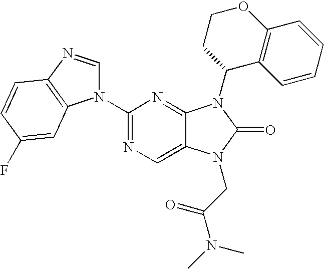 |
| | 728 | 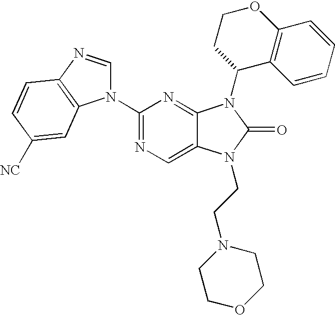 |
| | 729 | 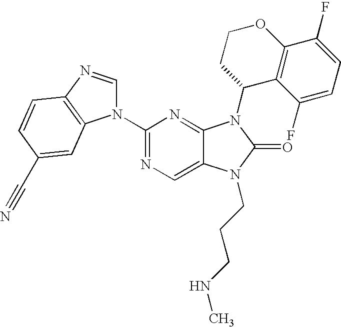 |
| | 730 | 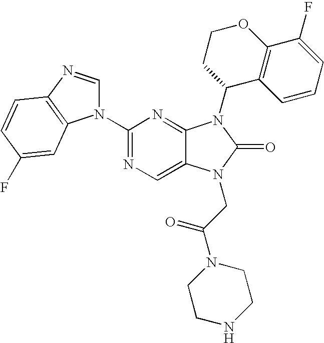 |
| | 731 | 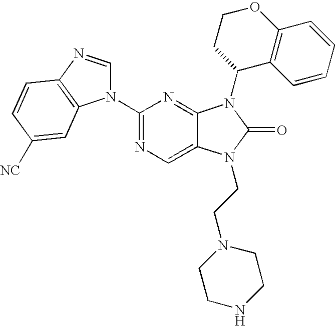 |
| | 732 | 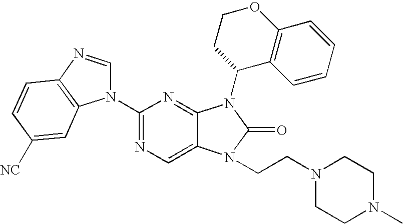 |
| | 733 | 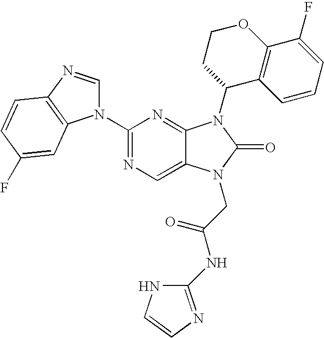 |
| | 734 | 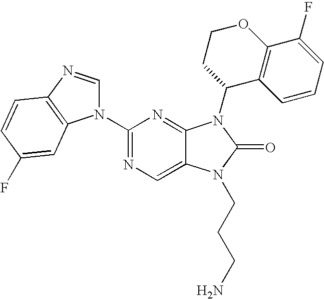 |
| | 735 | 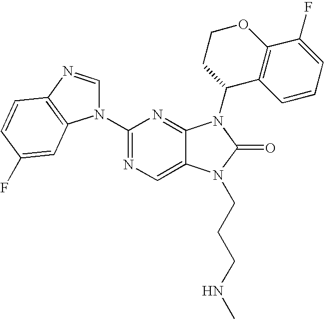 |
| | 736 | 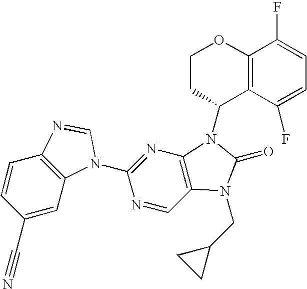 |
| | 737 | 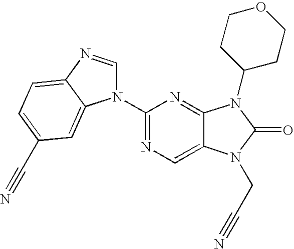 |
| | 738 | 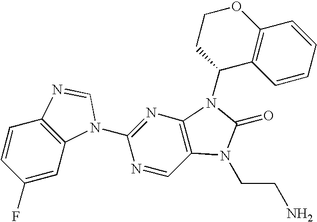 |
| | 739 | 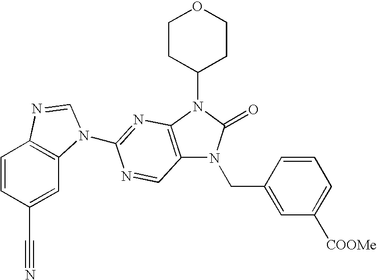 |
| | 740 | 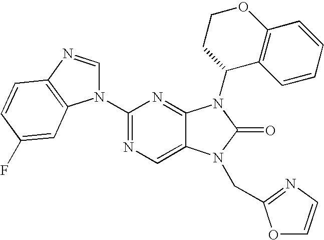 |
| | 741 | 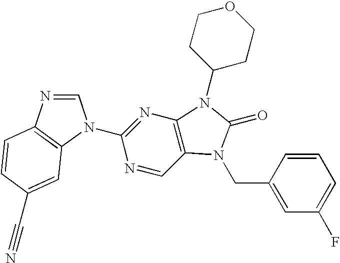 |
| | 742 | 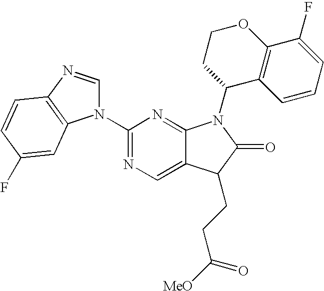 |
| | 743 | 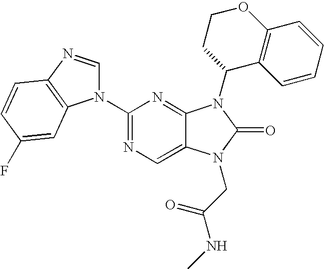 |
| | 744 | 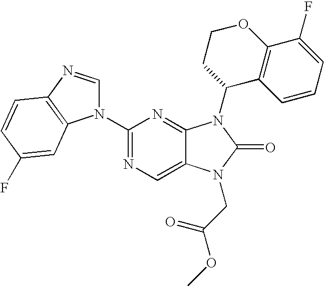 |
| | 745 | 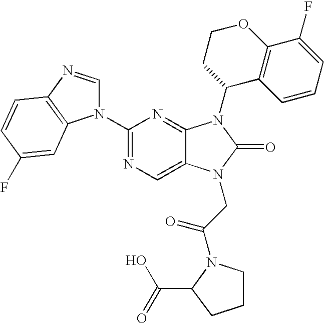 |
| | 746 | 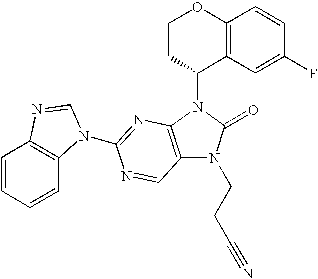 |
| | 747 | 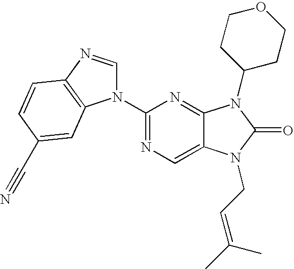 |
| | 748 | 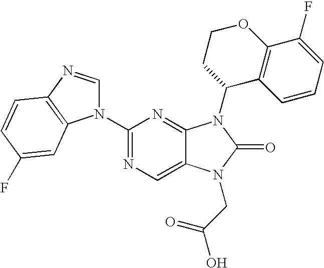 |
| | 749 | 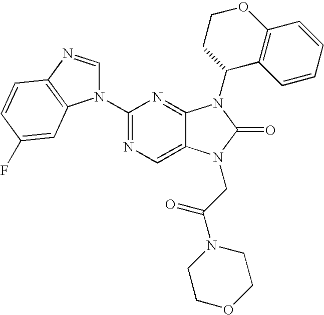 |
| | 750 | 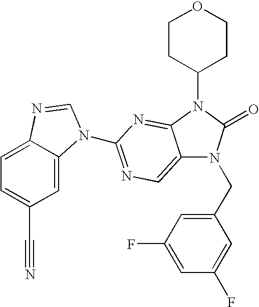 |
| | 751 | 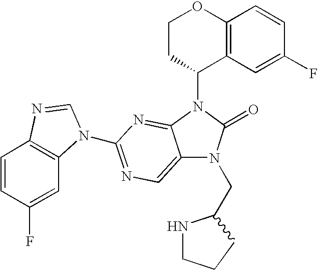 |
| | 752 | 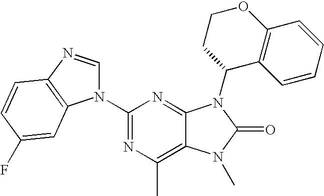 |
| | 753 | 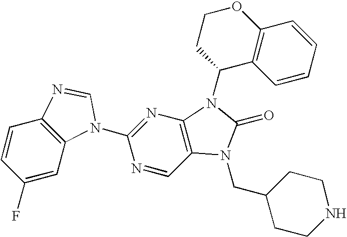 |
| | 754 | 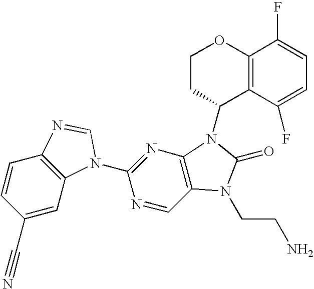 |
| | 755 | 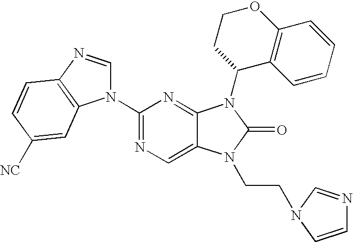 |
| | 756 | 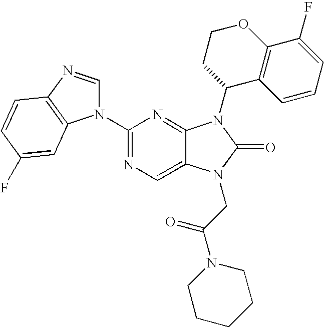 |
| | 757 | 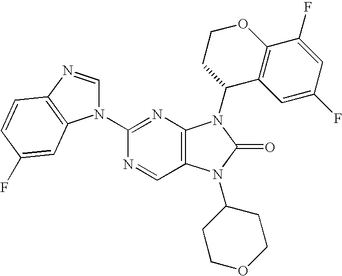 |
| | 758 | 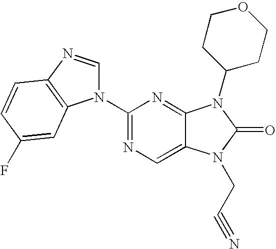 |
| | 759 | 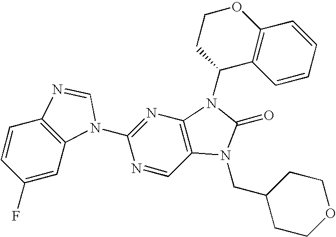 |
| | 760 | 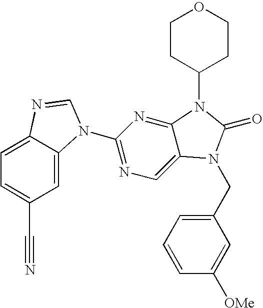 |
| | 761 | 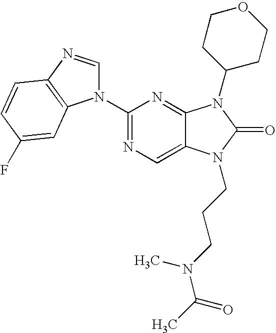 |
| | 762 | 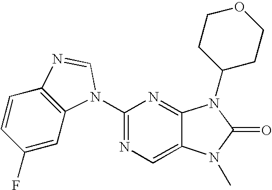 |
| | 763 | 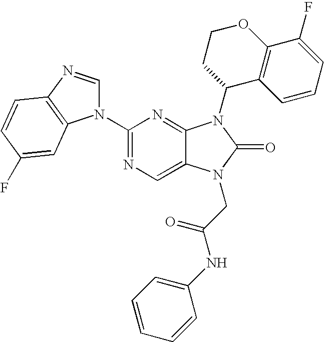 |
| | 764 | 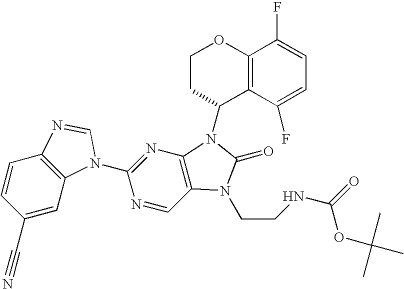 |
| | 765 | 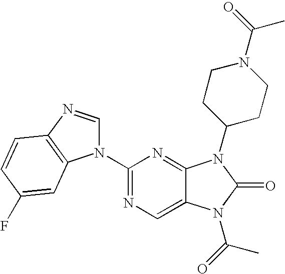 |

TABLE 2

| Synthesis Example | Reference No. | |
|---|---|---|
| | 601 | 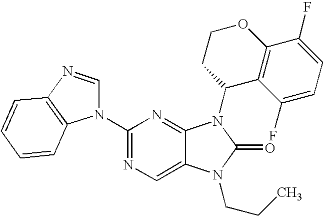 |
| | 766 | 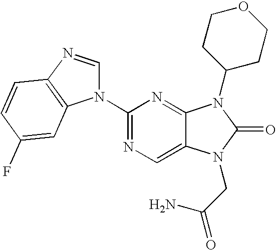 |
| | 767 | 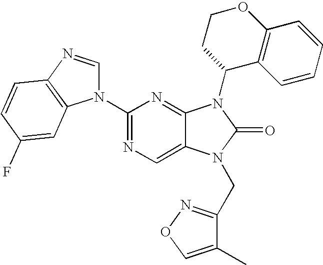 |
| | 768 | 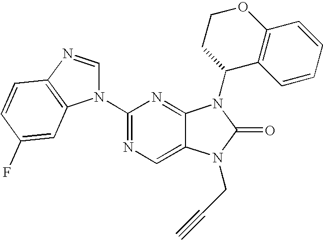 |
| | 769 | 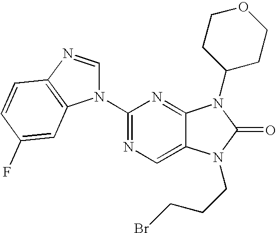 |
| | 770 | 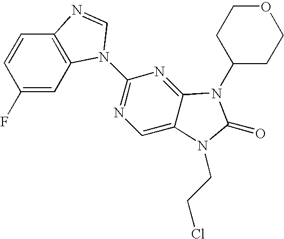 |
| | 771 | 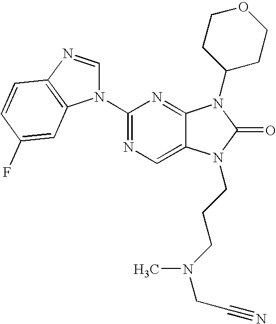 |
| | 772 | 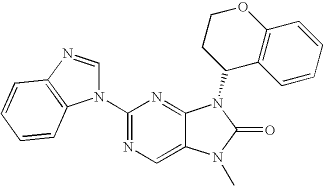 |
| | 773 | 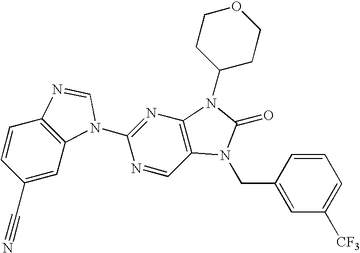 |
| | 774 | 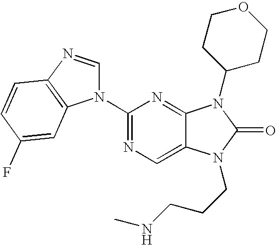 |
| | 775 | 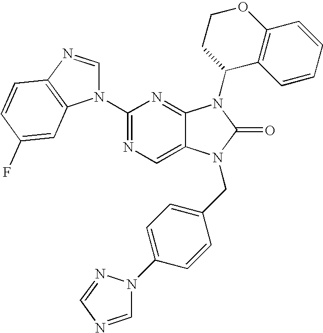 |
| | 776 | 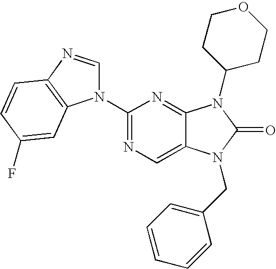 |
| | 778 | 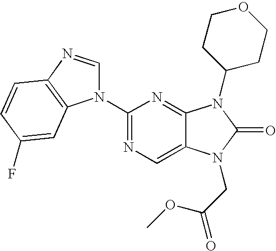 |
| | 779 | 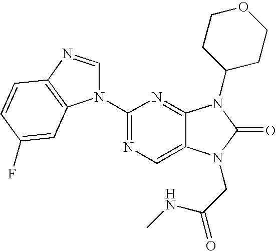 |
| | 780 | 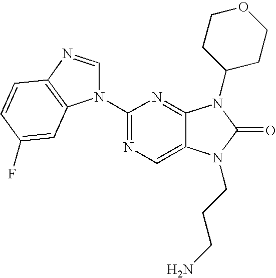 |
| | 781 | 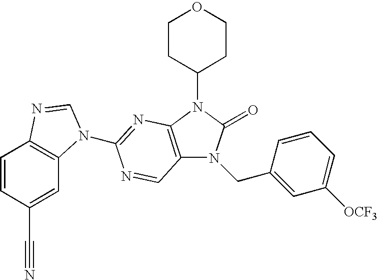 |
| | 783 | 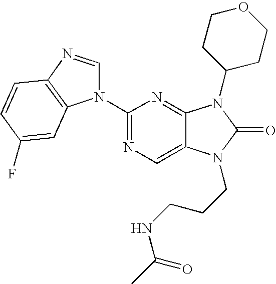 |
| | 784 | 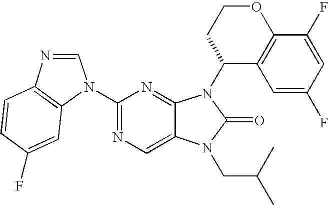 |
| | 785 | 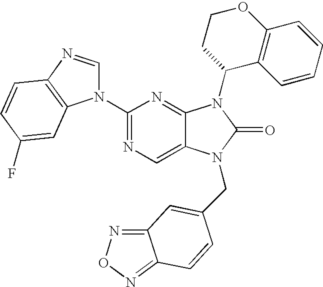 |
| | 786 | 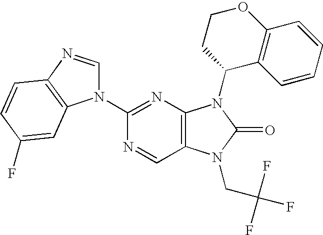 |
| 45 | 787 | 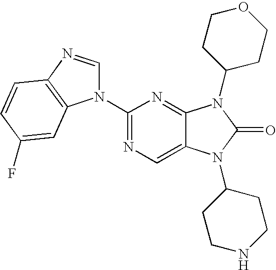 |
| | 788 | 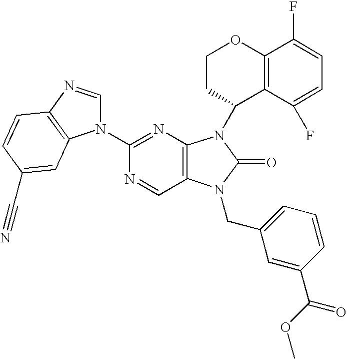 |
| | 789 | 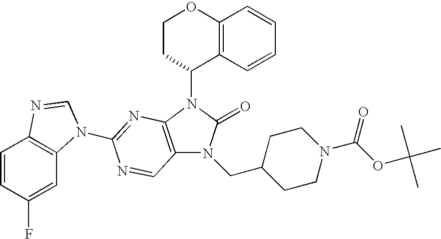 |
| | 790 | 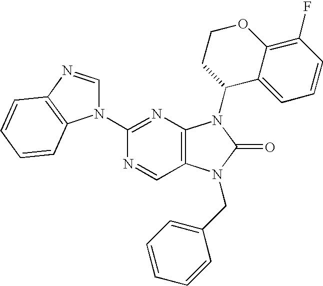 |
| | 791 | 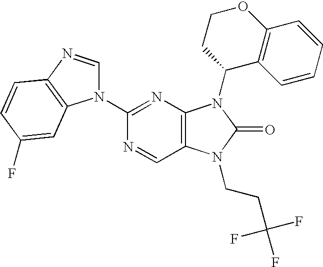 |
| | 792 | 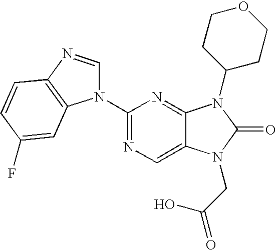 |

### IL-2-Induced IFN-γ Production in the Mouse

Administration of IL-2 leads to an increase in serum IFN-γ in the mouse due to NK secretion of the cytokine (Thornton S, Kuhn K A, Finkelman F D and Hirsch R. NK cells secrete high levels of IFN-γ in response to in vivo administration of IL-2. Eur J Immunol 2001 31:3355-3360). The experiment was carried out essentially according to the protocol in Thornton et al. and the test compounds were administered in order to determine the level of inhibition attained. In summary, female BALB/c mice were fasted for 12-18 hours before a study but had free access to water at all times. Test compounds were administered by gavage one hour before intraperitoneal injection of IL-2 and capture antibody. At termination of the studies, the mice were sacrificed by carbon dioxide inhalation, terminal blood samples were collected by cardiac puncture and serum was generated. Serum was stored frozen until it was assayed for IFN-γ, as described by the kit manufacturer (BD Pharmingen™, San Diego, Calif.).

Using the above method, the compounds of experimental examples 16, 28, and Table 1 examples 501, 504 and 505, at 30 mg/kg, were shown to inhibit IL-2-induced IFN-gamma production by >40% in vivo in the mouse. A reference compound, CP690550, exhibited 96% inhibition at 30 mg/kg in this screen.

The 7-substituted purinones exhibit increased selectivity for Jak3 compared to their 7-unsubstituted congeners.

Aurora A kinase assay was performed using a fluorescence polarization format. A 100 nM solution of fluorescien-labeled FAM PKAtide (Molecular Devices), the substrate for Aurora A (Upstate Biotechnology), was incubated with Aurora A (80 ng/ml) and 30 mM ATP at room temperature for 1 hour in the presence of an appropriate concentration of test inhibitor. The reaction was terminated by adding IMAP Progressive Binding Reagent mix according to the manufacturer's instructions (Molecular Devices). The polarization signal was detected using Aquest (Molecular Devices).

Some comparative examples are shown below. On the left is the 7-unsubstituted compound and its ratio of Jak3 to Aurora A inhibition given as “x-fold” selectivity. On the right are shown the 7-substituted congeners along with their selectivity. All of the IC₅₀'s for Jak3 are below 100 nM.

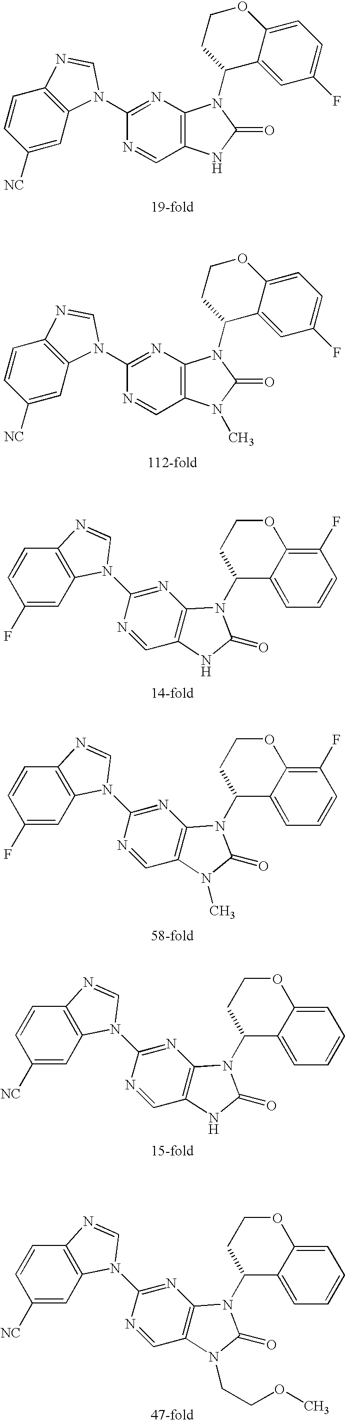

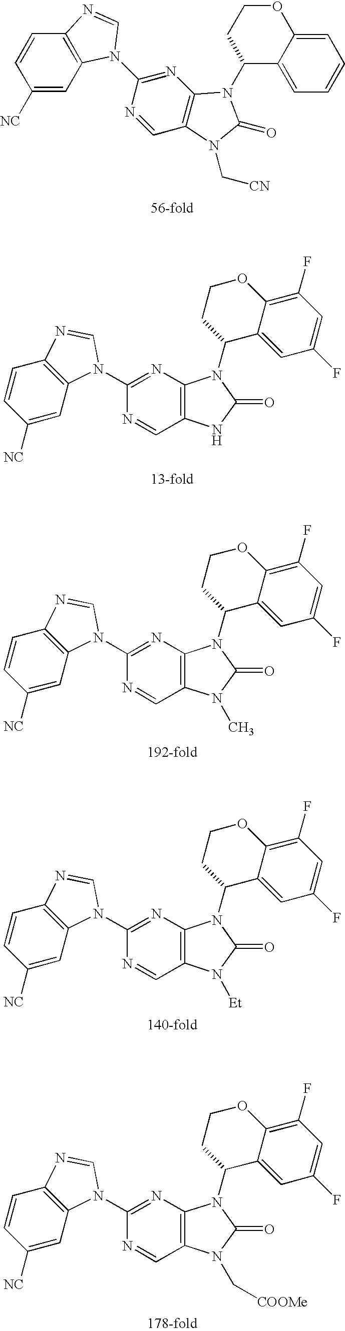

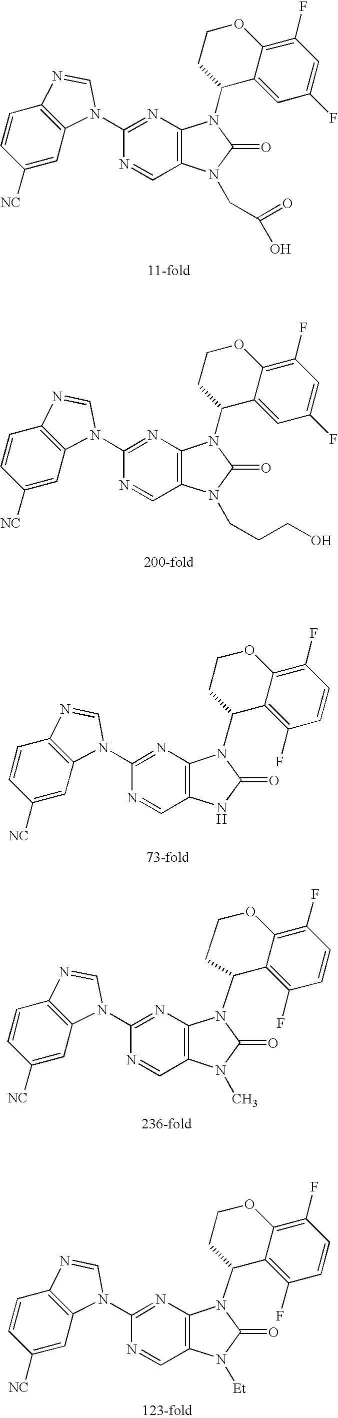

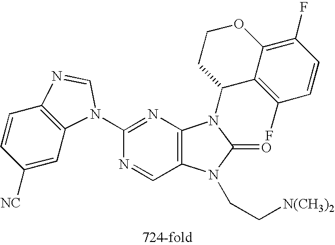

Although the foregoing invention has been described in some detail for purposes of illustration, it will be readily apparent to one skilled in the art that changes and modifications may be made without departing from the scope of the invention described herein.

1. A compound of formula III 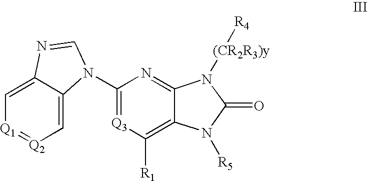
wherein
Q₁ and Q₂ are independently selected from the group consisting of CX₁, CX₂ and nitrogen wherein Q₁ and Q₂ are not both nitrogen;
Q₃ is N or CH;
X₁ and X₂ are independently selected from the group consisting of hydrogen, (C₁-C₆)alkyl, cyano, halo, halo(C₁-C₆)alkyl, hydroxyl, (C₁-C₆)alkoxy; halo(C₁-C₆)alkoxy, and nitro;
R₁ is selected from the group consisting of hydrogen and (C₁-C₆)alkyl;
y is zero or an integer selected from 1, 2 and 3;
R₂ and R₃ are selected independently for each occurrence of (CR₂R₃) from the group consisting of hydrogen and (C₁-C₆)alkyl;
R₄ is selected from a group consisting of alkyl, heterocyclyl, aryl, heteroaryl, substituted alkyl, substituted heterocyclyl, substituted aryl, substituted heteroaryl; and
R₅ is selected from the group consisting of alkyl, heterocyclyl, substituted heterocyclyl, and C₁-C₆ alkyl wherein
(a) one or two CH₂ is replaced by a group chosen from NH and N(alkyl);
(b) one or two CH₂ is replaced by O;
(c) one or two CH₂ is replaced by (C═O);
(d) two CH₂ are replaced by CH═CH or C≡C; or
(e) any chemically stable combination of (a), (b) (c) and (d);
and wherein from zero to three hydrogens is replaced by a substituent chosen from:
(a) halogen, hydroxy, cyano, loweralkylsulfonyl, loweralkylsulfonyloxy, amino, loweralkylamino, diloweralkylamino, alkoxyamino, sulfonylamino, acylamino, arylamino, loweralkoxy;
(b) heterocyclyl and heterocyclyl substituted with from one to three substituents chosen from halogen, hydroxy, alkoxy, alkyl and alkoxycarbonyl;
(c) phenyl and phenyl substituted with from one to three substituents chosen from halogen, hydroxy, alkoxy, alkyl, acylamino, cyano, carboxy, alkoxycarbonyl, haloalkyl and heterocyclyl; and
(d) heteroaryl and heteroaryl substituted with from one to three substituents chosen from halogen, hydroxy, alkoxy, alkyl and alkoxycarbonyl.

2. A compound according to claim 1 of formula:

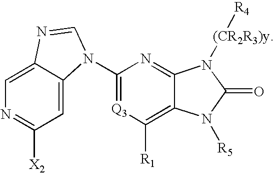

3. A compound according to claim 2 of formula:

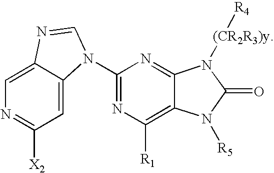

4. A compound according to claim 2 of formula:

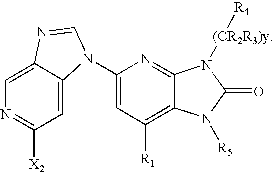

5. A compound according to claim 1 of formula:

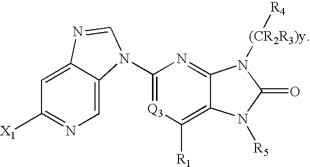

6. A compound according to claim 5 of formula:

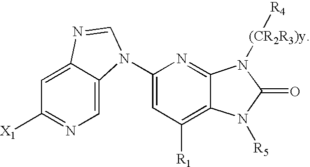

7. A compound according to claim 5 of formula:

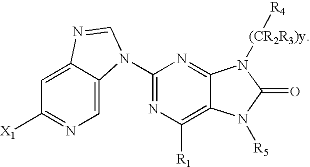

8. A compound according to claim 1 of formula:

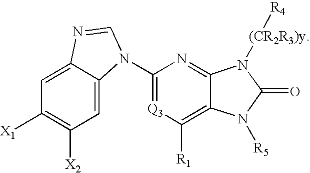

9. A compound according to claim 8 of formula:

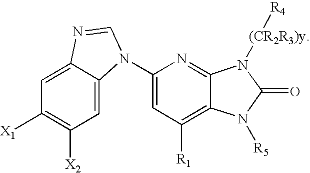

10. A compound according to claim 8 of formula:

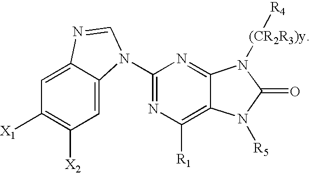

11. A compound according to claim 1 wherein X₁ and X₂ are independently selected from hydrogen, cyano, chloro, fluoro, methyl, trifluoromethyl and trifluoromethoxy.

12. A compound according to claim 1 wherein R₁ is H.

13. A compound according to claim 1 wherein, y is 1 or 2 and R₂ and R₃ are hydrogen or methyl.

14. A compound according to claim 13 wherein R₄ is selected from phenyl, quinoline, pyridine, pyrazine and their substituted counterparts.

15. A compound according to claim 1 wherein y is zero.

16. A compound according to claim 15 wherein R₄ is selected from cyclopentyl, cyclohexyl, phenyl, indane, tetralin, piperidine, oxepane, benzoxepane dihydrocyclopentapyridine, tetrahydropyran, tetrahydrofuran, tetrahydroindole, isoquinoline, tetrahydroisoquinoline, quinoline, tetrahydroquinoline, chroman, pyridine, pyrimidine, dihydropyran, dihydrobenzofuran, tetrahydrobenzofuran, tetrahydrobenzothiophene, furan, dihydropyrano[2,3-b]pyridine, tetrahydroquinoxaline, tetrahydrothiopyran (thiane), thiochroman (dihydrobenzothiin) and their substituted counterparts.

17. A compound according to claim 1 wherein,
(a) y is zero and R₄ is selected from cyclohexyl, tetralin, indane, oxepane, benzoxepane, dihydrocyclopentapyridine, tetrahydropyran, tetrahydroquinoline, chroman, dihydrobenzofuran, tetrahydrobenzofuran, dihydropyrano[2,3-b]pyridine and tetrahydroquinoxaline, each optionally substituted with hydroxy, oxo, or halogen; or
(b) y is 1 or 2, R₂ and R₃ are hydrogen or methyl and R₄ is selected from phenyl, pyridine and pyrazine, each optionally substituted with halogen.

18. A compound according to claim 17 wherein y is 0 and R₄ is chosen from chroman-4-yl; 3,4-dihydronaphthalen-1(2H)-on-4-yl; 2,3-dihydroinden-1-on-4-yl and their fluoro substituted counterparts.

19. A compound according to claim 18 wherein R₄ is chroman-4-yl and the carbon at 4 of the chroman is of the (R) configuration.

20. A compound according to claim 17 wherein y is 0 and R₄ is 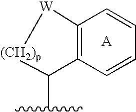
wherein
W is CH₂, C═O or O;
p is 1, 2 or 3;
A is a six-membered heteroaromatic ring containing 1 or 2 nitrogens or a benzene ring optionally substituted with one or two fluorines; and
the wavy line is the point of attachment to the purinone.

21. A compound according to claim 20 wherein the carbon marked with an asterisk 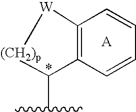
is of the (R) configuration.

22. A compound according to claim 17 wherein y is 1 and R₄ is selected from difluorophenyl, fluorophenyl, chlorophenyl, chlorofluorophenyl, pyridin-3-yl and pyrazin-3-yl.

23. A compound according to claim 17 wherein y is zero and R₄ is selected from tetrahydropyran-4-yl, 4-hydroxycyclohexyl, 4-oxocyclohexyl and oxepan-4-yl.

24. A compound according to claim 10 wherein X₁ and X₂ are independently selected from hydrogen, cyano, chloro and fluoro and R₁ is H.

25. A compound according to claim 24 wherein,
(a) y is zero and R₄ is selected from cyclohexyl, tetralin, indane, oxepane, benzoxepane, dihydrocyclopentapyridine, tetrahydropyran, tetrahydroquinoline, chroman, dihydrobenzofuran, tetrahydrobenzofuran, dihydropyrano[2,3-b]pyridine and tetrahydroquinoxaline, each optionally substituted with hydroxy, oxo, or halogen; or
(b) y is 1 or 2, R₂ and R₃ are hydrogen or methyl and R₄ is selected from phenyl, pyridine and pyrazine, each optionally substituted with halogen.

26. A compound according to claim 25 wherein R₅ is C₁-C₆ alkyl wherein
(a) one or two CH₂ is replaced by a group chosen from NH and N(alkyl);
(b) one or two CH₂ is replaced by O;
(c) one or two CH₂ is replaced by (C═O);
(d) two CH₂ are replaced by CH═CH or C≡C; or
(e) any chemically stable combination of (a), (b) (c) and (d);
and wherein from zero to three hydrogens is replaced by a substituent chosen from:
(a) halogen, hydroxy, cyano, loweralkylsulfonyl, loweralkylsulfonyloxy, amino, loweralkylamino, diloweralkylamino, alkoxyamino, sulfonylamino, acylamino, arylamino, loweralkoxy;
(b) heterocyclyl and heterocyclyl substituted with from one to three substituents chosen from halogen, hydroxy, alkoxy, alkyl and alkoxycarbonyl;
(c) phenyl and phenyl substituted with from one to three substituents chosen from halogen, hydroxy, alkoxy, alkyl, acylamino, cyano, carboxy, alkoxycarbonyl, haloalkyl and heterocyclyl; and
(d) heteroaryl and heteroaryl substituted with from one to three substituents chosen from halogen, hydroxy, alkoxy, alkyl and alkoxycarbonyl.

27. A compound according to claim 26 wherein y is 0 and R₄ is 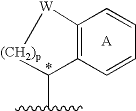
wherein the carbon marked with an asterisk is of the (R) configuration
W is CH₂, C═O or O;
p is 1, 2 or 3;
A is a six-membered heteroaromatic ring containing 1 or 2 nitrogens or a benzene ring optionally substituted with one or two fluorines; and
the wavy line is the point of attachment to the purinone.

28. A compound according to claim 27 wherein X₁ is hydrogen, X₂ is a substituent at the 6 position of the benzimidazole, and X₂ is chosen from hydrogen, fluoro and cyano.

29. A compound according to claim 1 wherein R₅ is C₁-C₆ alkyl or C₁-C₆ fluoroalkyl.

30. A compound according to claim 29 wherein y is 0 and R₄ is 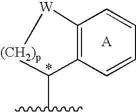
wherein the carbon marked with an asterisk is of the (R) configuration
W is CH₂, C═O or O;
p is 1, 2 or 3;
A is a six-membered heteroaromatic ring containing 1 or 2 nitrogens or a benzene ring optionally substituted with one or two fluorines; and
the wavy line is the point of attachment to the purinone.

31. A compound according to claim 1 wherein R₅ is C₁-C₆ alkyl and wherein from zero to three hydrogens is replaced by a substituent chosen from: hydroxy, carboxy, cyano, loweralkylsulfonyl, loweralkylsulfonyloxy, amino, loweralkylamino, diloweralkylamino, alkoxyamino, sulfonylamino, acylamino, arylamino and loweralkoxy.

32. A compound according to claim 31 wherein y is 0 and R₄ is 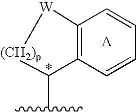
wherein the carbon marked with an asterisk is of the (R) configuration
W is CH₂, C═O or O;
p is 1, 2 or 3;
A is a six-membered heteroaromatic ring containing 1 or 2 nitrogens or a benzene ring optionally substituted with one or two fluorines; and
the wavy line is the point of attachment to the purinone.

33. A pharmaceutical composition comprising a pharmaceutically acceptable carrier and a therapeutically effective amount of at least one compound according to claim 1.

34. A method of treating a disorder which is dependent upon inhibition of Janus kinase 3, which comprises administering to a subject in need of such treatment a therapeutically effective amount of a compound according to a compound of formula III 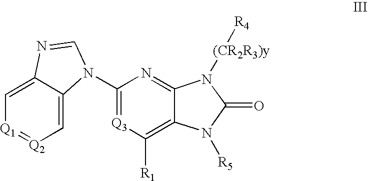
wherein
Q₁ and Q₂ are independently selected from the group consisting of CX₁, CX₂ and nitrogen wherein Q₁ and Q₂ are not both nitrogen;
Q₃ is N or CH;
X₁ and X₂ are independently selected from the group consisting of hydrogen, (C₁-C₆)alkyl, cyano, halo, halo(C₁-C₆)alkyl, hydroxyl, (C₁-C₆)alkoxy; halo(C₁-C₆)alkoxy, and nitro;
R₁ is selected from the group consisting of hydrogen and (C₁-C₆)alkyl;
y is zero or an integer selected from 1, 2 and 3;
R₂ and R₃ are selected independently for each occurrence of (CR₂R₃) from the group consisting of hydrogen and (C₁-C₆)alkyl;
R₄ is selected from a group consisting of alkyl, heterocyclyl, aryl, heteroaryl, substituted alkyl, substituted heterocyclyl, substituted aryl, substituted heteroaryl; and
R₅ is selected from the group consisting of alkyl, heterocyclyl, substituted heterocyclyl, and C₁-C₆ alkyl wherein
(a) one or two CH₂ is replaced by a group chosen from NH and N(alkyl);
(b) one or two CH₂ is replaced by O;
(c) one or two CH₂ is replaced by (C═O);
(d) two CH₂ are replaced by CH═CH or C≡C; or
(e) any chemically stable combination of (a), (b) (c) and (d);
and wherein from zero to three hydrogens is replaced by a substituent chosen from:
(a) halogen, hydroxy, cyano, loweralkylsulfonyl, loweralkylsulfonyloxy, amino, loweralkylamino, diloweralkylamino, alkoxyamino, sulfonylamino, acylamino, arylamino, loweralkoxy;
(b) heterocyclyl and heterocyclyl substituted with from one to three substituents chosen from halogen, hydroxy, alkoxy, alkyl and alkoxycarbonyl;
(c) phenyl and phenyl substituted with from one to three substituents chosen from halogen, hydroxy, alkoxy, alkyl, acylamino, cyano, carboxy, alkoxycarbonyl, haloalkyl and heterocyclyl; and
(d) heteroaryl and heteroaryl substituted with from one to three substituents chosen from halogen, hydroxy, alkoxy, alkyl and alkoxycarbonyl.

35. A method of treating a disorder which is dependent upon inhibition of Janus kinase 3, which comprises administering to a subject in need of such treatment a therapeutically effective amount of a compound formula III 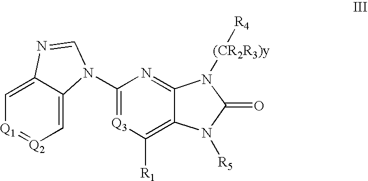
wherein
Q₁ and Q₂ are independently selected from the group consisting of CX₁, CX₂ and nitrogen wherein Q₁ and Q₂ are not both nitrogen;
Q₃ is N or CH;
X₁ and X₂ are independently selected from the group consisting of hydrogen, cyano, chloro, and fluoro;
R₁ is hydrogen;
y is zero or an integer selected from 1, 2 and 3;
R₂ and R₃ are selected independently for each occurrence of (CR₂R₃) from the group consisting of hydrogen and (C₁-C₆)alkyl;
R₄ is selected from a group consisting of alkyl, heterocyclyl, aryl, heteroaryl, substituted alkyl, substituted heterocyclyl, substituted aryl, substituted heteroaryl; and
R₅ is selected from the group consisting of alkyl, heterocyclyl, substituted heterocyclyl, and C₁-C₆ alkyl wherein
(a) one or two CH₂ is replaced by a group chosen from NH and N(alkyl);
(b) one or two CH₂ is replaced by O;
(c) one or two CH₂ is replaced by (C═O);
(d) two CH₂ are replaced by CH═CH or C≡C; or
(e) any chemically stable combination of (a), (b) (c) and (d);
and wherein from zero to three hydrogens is replaced by a substituent chosen from:
(a) halogen, hydroxy, cyano, loweralkylsulfonyl, loweralkylsulfonyloxy, amino, loweralkylamino, diloweralkylamino, alkoxyamino, sulfonylamino, acylamino, arylamino, loweralkoxy;
(b) heterocyclyl and heterocyclyl substituted with from one to three substituents chosen from halogen, hydroxy, alkoxy, alkyl and alkoxycarbonyl;
(c) phenyl and phenyl substituted with from one to three substituents chosen from halogen, hydroxy, alkoxy, alkyl, acylamino, cyano, carboxy, alkoxycarbonyl, haloalkyl and heterocyclyl; and
(d) heteroaryl and heteroaryl substituted with from one to three substituents chosen from halogen, hydroxy, alkoxy, alkyl and alkoxycarbonyl.

36. The method according to claim 34 wherein said disorder is selected from an autoimmune disease, an inflammatory disease, a mast cell mediated disease, hematological malignancy and organ transplant rejection.

37. The method according to claim 36 wherein said disorder is bone marrow transplant rejection.

38. The method according to claim 36 wherein said hematological malignancy is selected from leukemia and lymphoma.

39. The method according to claim 36 wherein said disorder is asthma.

40. The method according to claim 36 wherein said autoimmune disease is selected from an organ specific and a non-organ specific autoimmune disease.

41. The method according to claim 36 wherein said disorder is keratoconjuctivitis sicca.

42. The method according to claim 36 wherein said hematological malignancy is chronic myelogenous leukemia.

43. The method according to claim 34 wherein said disorder is selected from a leukemic form of cutaneous T-cell form lymphoma and acute lymphoblastic leukemia.

44. A method for treating a disorder selected from an autoimmune disease, an inflammatory disease, a mast cell mediated disease, hematological malignancy and organ transplant rejection, which comprises administering to a subject in need of such treatment a therapeutically effective amount of a compound according to a compound of formula III 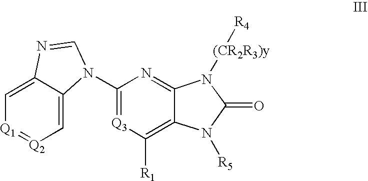
wherein
Q₁ and Q₂ are independently selected from the group consisting of CX₁, CX₂ and nitrogen wherein Q₁ and Q₂ are not both nitrogen;
Q₃ is N or CH;
X₁ and X₂ are independently selected from the group consisting of hydrogen, (C₁-C₆)alkyl, cyano, halo, halo(C₁-C₆)alkyl, hydroxyl, (C₁-C₆)alkoxy; halo(C₁-C₆)alkoxy, and nitro;
R₁ is selected from the group consisting of hydrogen and (C₁-C₆)alkyl;
y is zero or an integer selected from 1, 2 and 3;
R₂ and R₃ are selected independently for each occurrence of (CR₂R₃) from the group consisting of hydrogen and (C₁-C₆)alkyl;
R₄ is selected from a group consisting of alkyl, heterocyclyl, aryl, heteroaryl, substituted alkyl, substituted heterocyclyl, substituted aryl, substituted heteroaryl; and
R₅ is selected from the group consisting of alkyl, heterocyclyl, substituted heterocyclyl, and C₁-C₆ alkyl wherein
(a) one or two CH₂ is replaced by a group chosen from NH and N(alkyl);
(b) one or two CH₂ is replaced by O;
(c) one or two CH₂ is replaced by (C═O);
(d) two CH₂ are replaced by CH═CH or C≡C; or
(e) any chemically stable combination of (a), (b) (c) and (d);
and wherein from zero to three hydrogens is replaced by a substituent chosen from:
(a) halogen, hydroxy, cyano, loweralkylsulfonyl, loweralkylsulfonyloxy, amino, loweralkylamino, diloweralkylamino, alkoxyamino, sulfonylamino, acylamino, arylamino, loweralkoxy;
(b) heterocyclyl and heterocyclyl substituted with from one to three substituents chosen from halogen, hydroxy, alkoxy, alkyl and alkoxycarbonyl;
(c) phenyl and phenyl substituted with from one to three substituents chosen from halogen, hydroxy, alkoxy, alkyl, acylamino, cyano, carboxy, alkoxycarbonyl, haloalkyl and heterocyclyl; and
(d) heteroaryl and heteroaryl substituted with from one to three substituents chosen from halogen, hydroxy, alkoxy, alkyl and alkoxycarbonyl.
